(19) United States
(12) Patent Application Publication
(10) Pub. No.: US 2004/0067999 A1
(43) Pub. Date: Apr. 8, 2004

(54) Carbazole derivatives and their use as neuropeptide y5 receptor ligands

(76) Inventors: Michael Howard Block, Cheshire (GB); Kevin Michael Foote, Cheshire (GB); Craig Samuel Donald, Cheshire (GB); Paul Schofield, Cheshire (GB)

Correspondence Address: ROPES & GRAY LLP, ONE INTERNATIONAL PLACE, BOSTON, MA, 02110-2624, US

(21) Appl. No.: 10/450,928

(22) PCT Filed: Dec. 17, 2001
(86) PCT No.: PCT/GB01/05577

(30) Foreign Application Priority Data
Dec. 22, 2000 (GB) 0031382.5
Sep. 11, 2001 (GB) 0121919.5

(51) Int. Cl. A61K 31/405; C07D 209/14

(52) U.S. Cl. 514/415; 548/503; 514/414; 548/465

(57) ABSTRACT

Compounds of formula (I): are described wherein R¹-R⁶ and m are as defined within. Processes for their preparation and their use as NPY 5 inhibitors is described.

This invention relates to compounds which antagonise the interaction between neuropeptide Y (NPY) and the neuropeptide Y5 (NPY-5) receptor sub-type. This invention also relates to processes for the manufacture of NPY-5 receptor antagonists or agonists, pharmaceutically acceptable salts thereof, and to novel pharmaceutical compositions of NPY-5 receptor antagonists or agonists.

NPY is a 36 amino acid polypeptide which is a member of the pancreatic polypeptide family of regulatory peptides with widespread distribution throughout the mammalian system. NPY is the most abundant neuropeptide in the central and peripheral nervous systems and has been shown to have powerful and complex effects on feeding, anxiety, circadian rhythms, reproduction, pituitary-adrenocortical axis function, memory retention, seizures, thermo-regulation, and cardiovascular and gastrointestinal functions. NPY interacts with a heterogeneous population of at least six receptor subtypes, Y₁-Y₆ which activate adenylate cyclase via a G-protein. For reviews of NPY see: CRC Critical Reviews in Neurobiology. (1988) 4, 97-135; Regulatory Peptides (1996) 62, 1-11.

One of the most striking actions of NPY is induction of feeding in a variety of vertebrate species. Direct injection of NPY into the hypothalamus of satiated rats can increase food intake up to 10-fold over a 4 hour period and NPY is the only known peptide which can cause animals to eat until they are obese. Recent studies on NPY have focused on the identification of the NPY receptor responsible for the regulation of feeding. The NPY-5 receptor has been identified as the receptor most closely matching a proposed appetite receptor. The functional role of this receptor was addressed by receptor blockade studies. Intra-cerebro-ventricular injection of NPY-5 receptor antisense oligodeoxynucleotides prevented the increase in hypothalamic NPY levels during food deprivation and inhibited fasting-induced food intake in rats [Schaffhauser et al (1997) Diabetes 46, 1792-1798]. Thus the NPY-5 receptor is a potential pharmacological target in the modulation of feeding disorders such as obesity. For reviews on the association between NPY and feeding see: Zimanyi et al (1998) Current Pharm Des 4, 349-66; Heinrichs et al (1998) Vitamins and Hormones 54, 51-66.

Obesity is a large and ever expanding problem in affluent societies, which has reachedepidemic proportions. According to the US Institute of Medicine, 59% of Americans are clinically obese or at least 20% above their ideal body weight. Obesity is associated with susceptibility to a number of other conditions e.g. non-insulin-dependent diabetes, hypertension, dysilpidaemia and coronary heart disease. These conditions lead to reduction in life expectancy and decreased quality of life. The overall financial burden of obesity is difficult to quantify but it has been estimated that in the US it may account for 6-8% of total healthcare expenditure.

Thus there is need for pharmaceutical agents which have efficacy in the treatment of eating disorders such as obesity, anorexia, bulimia and related disorders. Examples of “related disorders” are diabetes, dyslipidaemia, hypertension and sleep disturbances, particularly diabetes.

Modulation of NPY activity through antagonism at the NPY-5 receptor offers one potential target for pharmacological intervention in these conditions.

WO 00/63171 discloses tricyclic compounds that are NPY inhibitors. Surprisingly, the present inventors have found that a particular class of these compounds are potent NPY5 inhibitors, and in addition, they possess a beneficial toxicological profile that make them particularly suitable for administration to a warm blooded animal, such as man.

Accordingly, the present invention provides a compound of formula (I):

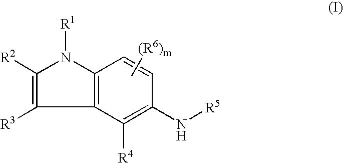

wherein:

R¹ is selected from C₁₋₄alkyl, C₁₋₄alkanoyl, C₁₋₄alkylsulphonyl, N-(C₁₋₄alkyl)sulphamoyl and N,N-(C₁₋₄alkyl)₂sulphamoyl wherein R¹ may be optionally substituted on carbon by one or more R⁷;

R² and R³ are both methyl or R² and R³ together form —(CH₂)₄— or —(CH)₄—; wherein said —(CH₂)₄— or —(CH)₄— may be optionally substituted by R⁸;

R⁴ is C₁₋₄alkyl;

R⁵ is —C(O)NR⁹R¹⁰, —C(O)R⁹ or —C(O)C(O)R⁹;

R⁶ and R⁸ are independently selected from halo, cyano, hydroxy, trifluoromethyl, trifluoromethoxy, C₁₋₄alkyl, N-(C₁₋₄alkyl)amino, N,N-(C₁₋₄alkyl)₂amino and C₁₋₄alkoxy;

R⁷ is halo, nitro, cyano, hydroxy, trifluoromethyl, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, C₁₋₄alylsulphonylamino, carbocyclyl or heterocyclyl;

R⁹ and R¹⁰ are independently hydrogen, C₁₋₁₀alkyl, C₂₋₁₀alkenyl, C₂₋₁₀alkynyl, C₁₋₄ alkoxy, carbocyclyl or heterocyclyl wherein R⁹ and R¹⁰ independently may be optionally substituted on carbon by one or more R¹¹; and wherein if said heterocyclyl contains an —NH— moiety that nitrogen may be optionally substituted by R¹²;

or R⁹ and R¹⁰ together with the nitrogen to which they are attached form a heterocyclic ring optionally substituted on carbon by one or more R¹³; and wherein if said heterocyclic ring contains an —NH— moiety that nitrogen may be optionally substituted by R¹⁴;

R¹¹ and R¹³ are independently selected from halo, hydroxy, cyano, ureido, amino, nitro, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, C₁₋₄alkanoylamino, C₂₋₆alkenyloxycarbonyl, C₁₋₄alkoxycarbonyl, N-(C₁₋₄alkyl)amino, N,N-(C₁₋₄alkyl)₂amino, C₁₋₄alkoxycarbonylamino, C₁₋₄alkoxycarbonyl-N-(C₁₋₄alkyl)amino, N-(C₁₋₄alkyl)carbamoyl, N,N-(C₁₋₄alkyl)₂carbamoyl, C₁₋₄alkylS(O)ₐ wherein a is 0-2, N-(C₁₋₄alkyl)sulphamoyl, N,N-(C₁₋₄alkyl)₂sulphamoyl, heterocyclyl, heterocyclyloxy, heterocyclylcarbonyl, heterocyclylcarbonylamino, heterocyclyloxycarbonyl, heterocyclylthio, carbocyclyl, carbocyclyloxy, carbocyclylcarbonyl, carbocyclylcarbonylamino, carbocyclyloxycarbonyl and carbocyclylthio; wherein R¹¹ and R¹³ independently may be optionally substituted on carbon by one or more R¹⁵; and wherein if said heterocyclyl contains an —NH— moiety that nitrogen may be optionally substituted by R¹⁶;

R¹², R¹⁴ and R¹⁶ are independently selected from C₁₋₄alkyl, C₁₋₄alkanoyl, C₁₋₄alkylsulphonyl, sulphamoyl, N-(C₁₋₄alkyl)sulphamoyl, N,N-(C₁₋₄alkyl)₂sulphamoyl, C₁₋₄alkoxycarbonyl, carbamoyl, N-(C₁₋₄alkyl)carbamoyl, N,N-(C₁₋₄alkyl)₂carbamoyl, carbocyclyl, carbocyclylC₁₋₄alkyl, carbocyclylcarbonyl, carbocyclylsulphonyl, heterocyclyl, heterocyclylC₁₋₄alkyl, heterocyclylcarbonyl, heterocyclylsulphonyl; wherein R¹², R¹⁴ and R¹⁶ independently may be optionally substituted on carbon by one or more R¹⁷;

R¹⁵ is selected from halo, hydroxy, cyano, carbamoyl, ureido, amino, nitro, carboxy, carbamoyl, mercapto, sulphamoyl, C₁₋₄alkyl, C₂₋₄alkenyl, C₂₋₄alkyl, C₁₋₄alkoxy, C₁₋₄alkanoyl, C₁₋₄alkanoyloxy, C₁₋₄alkanoylamino, C₂₋₆alkenyloxycarbonyl, C₁₋₄alkoxycarbonyl, N-(C₁₋₄alkyl)amino, N,N-(C₁₋₄alkyl)₂amino, C₁₋₄alkoxycarbonylamino, C₁₋₄alkoxycarbonyl-N-(C₁₋₄alkyl)amino, N-(C₁₋₄alykl)carbamoyl, N,N-(C₁₋₄alkyl)₂carbamoyl, C₁₋₄alkylS(O)ₐ wherein a is 0-2, N-(C₁₋₄alkyl)sulphamoyl, N,N-(C₁₋₄alkyl)₂sulphamoyl, heterocyclyl, heterocyclyloxy, heterocyclylcarbonyl, heterocyclylmethyloxy, heterocyclyloxycarbonyl, carbocyclyl, carbocyclyloxy, carbocyclylcarbonyl, carbocyclylmethyloxy and carbocyclyloxycarbonyl; wherein R¹⁵ may be optionally substituted on carbon by one or more R¹⁸;

R¹⁷ and R¹⁸ is selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, alkyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;

m is 0-2; wherein the values of R⁶ may be the same or different;

or a pharmaceutically acceptable salt, prodrug or solvate thereof.

In this specification the term “alkyl” includes both straight and branched chain alkyl groups but references to individual alkyl groups such as “propyl” are specific for the straight chain version only. For example, “C₁₋₁₀alkyl” and “C₁₋₄alkyl” includes propyl, isopropyl and t-butyl. However, references to individual alkyl groups such as ‘propyl’ are specific for the straight chained version only and references to individual branched chain alkyl groups such as ‘isopropyl’ are specific for the branched chain version only. A similar convention applies to other radicals, for example “phenylC₁₋₄alkyl” would include phenylC₁₋₄alkyl, benzyl, 1-phenylethyl and 2-phenylethyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A “heterocyclyl” is a saturated, partially saturated or unsaturated, mono or bicyclic ring containing 3-12 atoms of which at least one atom is chosen from nitrogen, sulphur or oxygen, which may, unless otherwise specified, be carbon or nitrogen linked, wherein a —CH₂— group can optionally be replaced by a —C(O)— or a ring sulphur atom may be optionally oxidised to form the S-oxides. Preferably a “heterocyclyl” is a saturated, partially saturated or unsaturated, mono or bicyclic ring containing 5 or 6 atoms of which at least one atom is chosen from nitrogen, sulphur or oxygen, which may, unless otherwise specified, be carbon or nitrogen linked, wherein a —CH₂— group can optionally be replaced by a —C(O)— or a ring sulphur atom may be optionally oxidised to form S-oxide(s). Examples and suitable values of the term “heterocyclyl” are thiazolidinyl, pyrrolidinyl, pyrrolinyl, 2-pyrrolidonyl, 2,5-dioxopyrrolidinyl, 2-benzoxazolinonyl, 1,1-dioxotetrahydrothienyl, 2,4-dioxoimidazolidinyl, 2-oxo-1,3,4-(4-triazolinyl), 2-oxazolidinonyl, 5,6-dihydrouracilyl, 1,3-benzodioxolyl, 1,2,4-oxadiazolyl, 2-azabicyclo[2.2.1]heptyl, 4-thiazolidonyl, morpholino, 2-oxotetrahydrofuranyl, tetrahydrofuranyl, 2,3-dihydrobenzofuranyl, benzothienyl, tetrahydropyranyl, piperidyl, 1-oxo-1,3-dihydroisoindolyl, piperazinyl, thiomorpholino, 1,1-dioxothiomorpholino, tetrahydropyranyl, 1,3-dioxolanyl, homopiperazinyl, thienyl, isoxazolyl, imidazolyl, pyrrolyl, thiadiazolyl, isothiazolyl, 1,2,4-triazolyl, 1,3,4-triazolyl, pyranyl, indolyl, pyrimidyl, thiazolyl, pyrazinyl, pyridazinyl, pyridyl, 4-pyridonyl, quinolyl and 1-isoquinolonyl. Preferably the term “heterocyclyl” refers to piperidinyl, tetrahydrofuran, morpholino, piperazinyl, 1,1-dioxotetrahydrothienyl, triazolyl, 2-pyrrolidinone, tetrahydropyran and pyridyl.

A “carbocyclyl” is a saturated, partially saturated or unsaturated, mono or bicyclic carbon ring that contains 3-12 atoms; wherein a —CH₂— group can optionally be replaced by a —C(O)—. Preferably “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. Particularly “carbocyclyl” is cyclohexyl.

An example of “C₁₋₄alkanoyloxy” is acetoxy. Examples of “C₁₋₄alkoxycarbonyl” include methoxycarbonyl, ethoxycarbonyl, n- and t-butoxycarbonyl. Examples of “C₁₋₄alkoxy” include methoxy, ethoxy and propoxy. Examples of “C₁₋₄alkanoylamino” include formamido, acetamido and propionylamino. Examples of “C₁₋₄alkylS(O)ₐ wherein a is 0 to 2” include methylthio, ethylthio, methylsulphinyl, ethylsulphinyl, mesyl and ethylsulphonyl. Examples of “C₁₋₄alkylsulphonyl” include mesyl and ethylsulphonyl. Examples of “C₁₋₄alkylsulphonylamino” include mesylamino and ethylsulphonylamino. Examples of “C₁₋₄alkanoyl” include propionyl and acetyl. Examples of “N-(C₁₋₄alkyl)amino” include methylamino and ethylamino. Examples of “N,N-(C₁₋₄alkyl)₂amino” include di-N-methylamino, di-(N-ethyl)amino and N-ethyl-N-methylamino. Examples of “C₂₋₁₀alkenyl” and “C₂₋₄alkenyl” are vinyl, alkyl and 1-propenyl. Examples of “C₂₋₁₀alkynyl” and “C₂₋₄alkynyl” are ethynyl, 1-propynyl and 2-propynyl. Examples of “N-(C₁₋₄alkyl)sulphamoyl” are N-(methyl)sulphamoyl and N-(ethyl)sulphamoyl. Examples of “N,N-(C₁₋₄alkyl)₂sulphamoyl” are N,N-(dimethyl)sulphamoyl and N-(methyl)-N-(ethyl)sulphamoyl. Examples of “N-(C₁₋₄alkyl)carbamoyl” are methylaminocarbonyl and ethylaminocarbonyl. Examples of “N,N-(C₁₋₄alkyl)₂carbamoyl” are dimethylaminocarbonyl and methylethylaminocarbonyl. Examples of “C₂₋₆alkenyloxycarbonyl” are alkyloxycarbonyl and 2-butenyloxycarbonyl. Examples of “C₁₋₄alkoxycarbonylamino are methoxycarbonylamino, ethoxycarbonylamino, n- and t-butoxycarbonylamino. Examples of “C₁₋₄alkoxycarbonyl-N-(C₁₋₄alkyl)amino are methoxycarbonyl-N-methylamino, ethoxycarbonyl-N-ethylamino, n- and t-butoxycarbonyl-N-methylamino. Examples of “heterocyclyloxy” are pyridyloxy and thiazolyloxy. Examples of “heterocyclylcarbonyl” are pyrimidylcarbonyl and morpholinocarbonyl. Examples of “heterocyclyloxycarbonyl” are pyrrolidinyloxycarbonyl and pyranyloxycarbonyl. Examples of “carbocyclyloxy” are phenoxy and cyclopropyloxy. Examples of “carbocyclylcarbonyl” are benzoyl and cyclohexylcarbonyl. Examples of “carbocyclyloxycarbonyl” are phenoxycarbonyl and indanyloxycarbonyl. Examples of “heterocyclylcarbonylamino” are morpholinocarbonylamino, pyridylcarbonylamino and theinylcarbonylamino. Examples of “heterocyclylthio” are pyridylthio, pyranylthio and pyrrolinylthio. Examples of “carbocyclylcarbonylamino” are benzoylamino and cyclopropylcarbonylamino. Examples of “carbocyclylthio” are phenylthio and cyclohexylthio. Examples of “heterocyclylmethyloxy” are pyridylmethyloxyl and piperidylmethyloxy. Examples of “carbocyclylmethyloxy” are benzyloxy and cyclopentylmethyloxy. Examples of “carbocyclylC₁₋₄alkyl” are phenethyl, benzyl and cyclopropylmethyl. Examples of “carbocyclylsulphonyl” are phenylsulphonyl and cyclohexylsulphonyl. Examples of “heterocyclylC₁₋₄alkyl” are pyridylmethyl and pyrrolidinonylethyl. Examples of “heterocyclylsulphonyl” are pyrazinylsylphonyl and morpholinosulphonyl.

A suitable pharmaceutically-acceptable salt of a compound of formula (I) is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically-acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

The compounds of the formula (I) may be administered in the form of a prodrug which is broken down in the human or animal body to give a compound of the formula (I). Examples of prodrugs include in vivo hydrolysable esters of a compound of the formula (I).

Various forms of prodrugs are known in the art. For examples of such prodrug derivatives, see:

a) Design of Prodrugs, edited by H. Bundgaard, (Elsevier, 1985) and Methods in Enzymology, Vol. 42, p. 309-396, edited by K. Widder, et al. (Academic Press, 1985);

b) A Textbook of Drug Design and Development, edited by Krogsgaard-Larsen and H. Bundgaard, Chapter 5 “Design and Application of Prodrugs”, by H. Bundgaard p. 113-191 (1991);

c) H. Bundgaard, Advanced Drug Delivery Reviews, 8, 1-38 (1992);

d) H. Bundgaard, et al., Journal of Pharmaceutical Sciences, 77, 285 (1988); and

e) N. Kakeya, et al., Chem Pharm Bull, 32, 692 (1984).

An in vivo hydrolysable ester of a compound of the formula (I) containing a carboxy or a hydroxy group is, for example, a pharmaceutically-acceptable ester which is hydrolysed in the human or animal body to produce the parent acid or alcohol. Suitable pharmaceutically-acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters, for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

An in vivo hydrolysable ester of a compound of the formula (I) containing a hydroxy group includes inorganic esters such as phosphate esters (including phosphoramidic cyclic esters) and α-acyloxyalkyl ethers and related compounds which as a result of the in vivo hydrolysis of the ester breakdown to give the parent hydroxy group/s. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

It is to be understood that, insofar as certain of the compounds of formula (I) defined above may exist in optically active or racemic forms by virtue of one or more asymmetric carbon atoms, the invention includes in its definition any such optically active or racemic form which possesses the property of being an agonist or antagonist at the neuropeptide Y5 receptor. The synthesis of optically active forms may be carried out by standard techniques of organic chemistry well known in the art, for example by synthesis from optically active starting materials or by resolution of a racemic form. Similarly, binding to the neuropeptide Y5 receptor may be evaluated using the standard laboratory techniques referred to hereinafter.

The invention also relates to any and all tautomeric forms of the compounds of the formula (I) that possess neuropeptide Y5 receptor agonist or antagonist activity.

It will also be understood that certain compounds of the present invention may exist in solvated, for example hydrated, as well as unsolvated forms. It is to be understood that the present invention encompasses all such solvated forms which possess the property of interacting with the neuropeptide Y5 receptor.

Preferred values of the variable groups are as follows. Such values may be used where appropriate with any of the definitions, claims or embodiments defined hereinbefore or hereinafter.

Preferably R¹ is selected from C₁₋₄alkyl optionally substituted on carbon by one or more R⁷ wherein R⁷ is C₁₋₄alkoxy.

More preferably R¹ is selected from ethyl, isopropyl or 2-methoxy-1-methylethyl.

In one aspect of the invention, particularly R¹ is ethyl.

In another aspect of the invention, particularly R¹ is isopropyl.

In one aspect of the invention, preferably R² and R³ are both methyl.

In another aspect of the invention, preferably R² and R³ together form —(CH₂)₄— optionally substituted by R⁸.

In a further aspect of the invention, preferably R² and R³ together form —(CH)₄— optionally substituted by R⁸.

Preferably R² and R³ together form —(CH₂)₄— or —(CH)₄— optionally substituted by R⁸; wherein R⁸ is selected from halo or C₁₋₄alkyl.

Preferably R² and R³ together form —(CH₂)₄— or —(CH)₄— optionally substituted by R⁸; wherein R⁸ is selected from fluoro, bromo or methyl.

Preferably R⁴ is methyl or isopropyl.

In one aspect of the invention, more preferably R⁴ is methyl.

In another aspect of the invention, more preferably R⁴ is isopropyl.

In one aspect of the invention, preferably R⁵ is —C(O)NR⁹R¹⁰.

In another aspect of the invention, preferably R⁵ is —C(O)R⁹.

In a further aspect of the invention, preferably R⁵ is —C(O)C(O)R⁹.

Preferably R⁵ is —C(O)NR⁹R¹⁰, —C(O)R⁹ or —C(O)C(O)R⁹; wherein

R⁹ and R¹⁰ are independently C₁₋₁₀alkyl, C₁₋₄alkoxy or heterocyclyl wherein R⁹ and R¹⁰ independently may be optionally substituted on carbon by one or more R¹¹; and wherein if said heterocyclyl contains an —NH— moiety that nitrogen may be optionally substituted by R¹²;

or R⁹ and R¹⁰ together with the nitrogen to which they are attached form a heterocyclic ring optionally substituted on carbon by one or more R¹³; and wherein if said heterocyclic ring contains an —NH— moiety that nitrogen may be optionally substituted by R¹⁴;

R¹¹ and R¹³ are independently selected from halo, hydroxy, carbamoyl, amino, C₁₋₄alkyl, C₁₋₄alkoxy, C₁₋₄alkoxycarbonylamino, heterocyclyl, carbocyclyl; wherein R¹¹ and R¹³ independently may be optionally substituted on carbon by one or more R¹⁵;

R¹² and R¹⁴ are independently selected from C₁₋₄alkyl, heterocyclyl;

R¹⁵ is selected from hydroxy, amino, C₁₋₄alkoxycarbonylamino.

More preferably R⁵ is —C(O)NR⁹R¹⁰, —C(O)R⁹ or —C(O)C(O)R⁹; wherein

R⁹ and R¹⁰ are independently C₁₋₄alkyl, C₁₋₄alkoxy, piperidinyl or tetrahydrofuran; wherein R⁹ and R¹⁰ independently may be optionally substituted on carbon by one or more R¹¹; and wherein if said heterocyclyl contains an —NH— moiety that nitrogen may be optionally substituted by R¹²;

or R⁹ and R¹⁰ together with the nitrogen to which they are attached form morpholino, piperidin-1-yl, piperazin-1-yl optionally substituted on carbon by one or more R¹³; and wherein piperazin-1-yl may be optionally substituted on nitrogen by R¹⁴;

R¹¹ and R¹³ are independently selected from chloro, hydroxy, carbamoyl, amino, C₁₋₄alkyl, C₁₋₄alkoxy, C₁₋₄alkoxycarbonylamino, 1,1-dioxotetrahydrothienyl, triazolyl, 2-pyrrolidinone, tetrahydropyran, pyridyl, cyclohexyl; wherein R¹¹ and R ¹³ independently may be optionally substituted on carbon by one or more R¹⁵;

R¹² and R¹⁴ are independently selected from C₁₋₄alkyl, pyridyl;

R¹⁵ is selected from hydroxy, amino, C₁₋₄alkoxycarbonylamino.

Particularly R⁵ is selected from t-butoxycarbonyl, morpholino,

tetrahydrofuran-3-ylcarbonyl, 2-(1,1-dioxotetrahydrothien-3-yl)acetyl,

3-carbamoylpiperidin-1-ylcarbonyl, 2,2,2-trichloroacetyl,

4-(pyrid-2-yl)piperazin-1-ylcarbonyl, 3-(pyrid-4-yl)propionyl,

N-[2-(pyrid-4-yl)ethyl]-N-methylaminocarbonyl,

N-(pyrid-3-ylmethyl)-N-methylaminocarbonyl,

N-(1-methylpiperid-4-yl)-N-methylaminocarbonyl, 4-hydroxymethylpiperid-1-ylcarbonyl,

4-(pyrid-4-yl)piperid-1-ylcarbonyl, 4-hydroxyethylpiperid-1-ylcarbonyl,

3-methoxy-2-(t-butoxycarbonylamino)propionyl, 2-(1,2,4-triazol-1-yl)acetyl,

2-(2-pyrrolidinon-1-yl)acetyl, tetrahydropyran-4-ylcarbonyl,

2-[3-(t-butoxycarbonylamino)cyclohexyl]acetyl, 3-methoxy-2-aminopropionyl,

2-(3-aminocyclohexyl)acetyl, 4-pyrid-4-ylpiperidin-1-yloxalyl,

4-pyrid-2-ylpiperazin-1-yloxalyl, 4hydroxypiperidin-1-yl, 2-hydroxyethylaminocarbonyl and

N-methoxy-N-methylaminocarbonyl.

Particularly R⁵ is selected from t-butoxycarbonyl, morpholinocarbonyl,

tetrahydrofuran-3-ylcarbonyl, 2-(1,1-dioxotetrahydrothien-3-yl)acetyl,

3-carbamoylpiperidin-1-ylcarbonyl, 2,2,2-trichloroacetyl,

4-(pyrid-2-yl)piperazin-1-ylcarbonyl, 3-(pyrid-4-yl)propionyl,

N-[2-(pyrid-4-yl)ethyl]-N-methylaminocarbonyl,

N-(pyrid-3-ylmethyl)-N-methylaminocarbonyl,

N-(1-methylpiperid-4-yl)-N-methylaminocarbonyl, 4-hydroxymethylpiperid-1-ylcarbonyl,

4-(pyridyl)piperid-1-ylcarbonyl, 4-hydroxyethylpiperid-1-ylcarbonyl,

3-methoxy-2-(t-butoxycarbonylamino)propionyl, 2-(1,2,4-triazol-1-yl)acetyl,

2-(2-pyrrolidinon-1-yl)acetyl, tetrahydropyran-4-ylcarbonyl,

2-[3-(t-butoxycarbonylamino)cyclohexyl]acetyl, 3-methoxy-2-aminopropionyl,

2-(3-aminocyclohexyl)acetyl, 4-pyrid-4-ylpiperidin-1-yloxalyl,

4-pyrid-2-ylpiperazin-1-yloxalyl, 4-hydroxypiperidin-1-yl, 2-hydroxyethylaminocarbonyl and

N-methoxy-N-methylaminocarbonyl.

In another aspect of the invention, preferably R⁵ is —C(O)NR⁹R¹⁰ or —C(O)R⁹;

R⁹ and R¹⁰ are independently hydrogen, C₁₋₁₀alkyl, carbocyclyl or heterocyclyl wherein R⁹ and R¹⁰ independently may be optionally substituted on carbon by one or more R¹¹; and wherein if said heterocyclyl contains an —NH— moiety that nitrogen may be optionally substituted by R¹²;

or R⁹ and R¹⁰ together with the nitrogen to which they are attached form a heterocyclic ring optionally substituted on carbon by one or more R¹³; and wherein if said heterocyclic ring contains an —NH— moiety that nitrogen may be optionally substituted by R¹⁴;

R¹¹ and R¹³ are independently selected from halo, hydroxy, carboxy, carbamoyl, C₁₋₄alkyl, C₁₋₄alkoxy, C₁₋₄alkanoylamino, C₁₋₄alkoxycarbonyl, N-(C₁₋₄alkyl)amino, N,N-(C₁₋₄alkyl)₂amino, C₁₋₄alkoxycarbonylamino, N-(C₁₋₄alkyl)carbamoyl, N,N-(C₁₋₄alkyl)₂carbamoyl, C₁₋₄alkylS(O)ₐ wherein a is 0 or 2, heterocyclyl, heterocyclyloxy or carbocyclyl; wherein R¹¹ and R¹³ independently may be optionally substituted on carbon by one or more R¹⁵;

R¹² and R¹⁴ are independently selected from C₁₋₄alkyl, C₁₋₄alkanoyl, carbamoyl, C₁₋₄alkoxycarbonyl, N-(C₁₋₄alkyl)carbamoyl, N,N-(C₁₋₄alkyl)₂carbamoyl and carbocyclylC₁₋₄alkyl;

R¹⁵ is selected from hydroxy, C₁₋₄alkoxy, N,N-(C₁₋₄alkyl)₂amino, heterocyclyl.

In another aspect of the invention, more preferably R⁵ is —C(O)NR⁹R¹⁰ or —C(O)R⁹;

R⁹ and R¹⁰ are independently hydrogen, C₁₋₆alkyl, cyclopropyl, 2-H-5,6-dihydropyranyl, 4-H-5,6-dihydropyranyl, 2-oxotetrahydrofuranyl, tetrahydrofuranyl, pyrrolidinyl, 2-oxopyrrolidinyl, pyrazinyl, 1,2,5,6-tetrahydropyridinyl, isoxazolyl, 1,2,4-triazolyl, tetrahydrothienyl, tetrahydropyranyl or piperidinyl wherein R⁹ and R¹⁰ independently may be optionally substituted on carbon by one or more R¹¹; and wherein if said heterocyclyl contains an —NH— moiety that nitrogen may be optionally substituted by R¹²;

or R⁹ and R¹⁰ together with the nitrogen to which they are attached form piperidinyl, pyrrolidinyl, homopiperazinyl, 4-oxohomopiperazinyl, morpholino, 2-oxopiperazinyl or piperazinyl optionally substituted on carbon by one or more R¹³; and wherein if said heterocyclic ring contains an —NH— moiety that nitrogen may be optionally substituted by R¹⁴;

R¹¹ and R¹³ are independently selected from halo, hydroxy, carboxy, carbamoyl, C₁₋₄alkyl, C₁₋₄alkoxy, C₁₋₄alkanoylamino, C₁₋₄alkoxycarbonyl, N-(C₁₋₄alkyl)amino, N,N-(C₁₋₄alkyl)₂amino, C₁₋₄alkoxycarbonylamino, N-(C₁₋₄alkyl)carbamoyl, N,N-(C₁₋₄alkyl)₂carbamoyl, C₁₋₄alkylS(O)ₐ wherein a is 0 or 2, piperidinyl, 2,4-dioxoimidazolidinyl, tetrahydrofuranyl, tetrahydrofuranyloxy, 4-oxo-1,4-dihydropyridinyl, pyrazinyl, cyclohexyl or phenyl; wherein R¹¹ and R¹³ independently may be optionally substituted on carbon by one or more R¹⁵;

R¹² and R¹⁴ are independently selected from C₁₋₄alkyl, C₁₋₄alkanoyl, carbamoyl, C₁₋₄alkoxycarbonyl, N-(C₁₋₄alkyl)carbamoyl, N,N-(C₁₋₄alkyl)₂carbamoyl and benzyl;

R¹⁵ is selected from hydroxy, C₁₋₄alkoxy, N,N-(C₁₋₄alkyl)₂amino, pyrrolininyl.

In another aspect of the invention, particularly R⁵ is

3-ethoxycarbonylpiperidin-1-ylcarbonyl, 3-hydroxymethylpiperidin-1-ylcarbonyl,

4-carbamoylpiperidin-1-ylcarbonyl, 4-ethoxycarbonylpiperidin-1-ylcarbonyl,

1-acetylpiperidin-4-ylcarbonyl, 1-N,N-dimethylcarbamoylpiperidin-3-ylcarbonyl,

1-acetylpiperidin-3-ylcarbonyl, 3-methoxycarbonylpyrrolidin-1-ylcarbonyl,

1-N-methylcarbamoylpiperidin-3-ylcarbonyl, 1-carbamoylpiperidin-3-ylcarbonyl,

3-N,N-dimethylcarbamoylpyrrolidin-1-ylcarbonyl, 1-carbamoylpiperidin-4-ylcarbonyl,

1-N,N-dimethylcarbamoylpiperidinylcarbonyl, 1-acetlypyrrolidin-3-ylcarbonyl,

1-N,N-dimethylcarbamoylpyrrolidin-3-ylcarbonyl,

1-N-methylcarbamoylpyrrolidin-3-ylcarbonyl, 1-hydroxy-3-methylpent-2-ylaminocarbonyl,

3-hydroxy-3-phenylprop-2-ylaminocarbonyl, (1-hydroxycyclohex-1-yl)methylaminocarbonyl,

N-methyl-N-(3-phenyl-3-hydroxyprop-2-yl)aminocarbonyl,

2-hydroxymethylpiperidin-1-ylcarbonyl, 2-hydroxymethylpyrrolidin-1-ylcarbonyl,

3-hydroxypiperidin-1-ylcarbonyl, 3-cyclohexyl-1-hydroxyprop-2-ylaminocarbonyl,

3-hydroxyprop-2-ylaminocarbonyl, 3-hydroxypyrrolidin-1-ylcarbonyl,

4-N,N-dimethylcarbamoylpiperidin-1-ylcarbonyl,

4-N-methylcarbamoylpiperidin-1-ylcarbonyl, 3-hydroxypent-3-ylcarbonylamino,

N,N-dimethylaminoacetyl, methoxyacetyl, ethoxyacetyl, 2-hydroxybut-2-ylcarbonyl,

N-methylpyrrolidin-2-ylcarbonyl, 2-oxotetrahydrofuran-5-ylcarbonyl,

tetrahydrofuran-2-ylcarbonyl, 2-hydroxy-1-acetamidoethyl, 3-methoxypropionyl,

2-methoxyethoxyacetyl, 3-ethoxypropionyl, 3-methylthiopropionyl,

2-trifluoromethyl-2-hydroxypropionyl, 2-phenyl-2-methoxypropionyl, piperidin-1-ylacetyl,

tetrahydrofuran-3-yloxyacetyl, tetrahydrothien-2-ylcarbonyl, 3-hydroxypropionyl,

(3-hydroxymethyltetrahydropyran-3-yl)carbonyl, (1-carbamoylcycloprop-1-yl)carbonyl,

3-acetamidopropionyl, 2-H-5,6-dihydropyran-3-ylcarbonyl, 3-ethylthioprop-2-ylcarbonyl,

2,3,4H-dihydpyran-2-ylcarbonyl, 1-hydroxy-3-methylbutylcarbonyl,

3-N,N-dimethylaminopyrrolidin-1-ylcarbonyl, 2-methyl-2-hydroxypropionyl,

N-methyl-N-(2-hydroxyethyl)aminocarbonyl, N,N-di-(2-hydroxyethyl)aminocarbonyl,

tetrahydrofuran-2-ylmethylaminocarbonyl, 2,6-dimethylpiperazin-4-ylcarbonyl,

4-hydroxypiperidin-1-ylcarbonyl, N-methyl-N-(N-methylpiperidinyl)aminocarbonyl,

2-methoxyprop-2-ylaminocarbonyl, 2-hydroxypropylaminocarbonyl,

2-methoxyethylaminocarbonyl, 2-methoxymethylpyrrolidin-1-ylcarbonyl,

2-(pyrrolidin-1-ylmethyl)pyrrolidin-1-ylcarbonyl, 3-hydroxypiperidin-1-ylcarbonyl,

N-methyl-N-(3-N,N-dimethylaminopropyl)aminocarbonyl,

N,N-di-(2-methoxyethyl)aminocarbonyl, 1-acetylpiperazin-4-ylcarbonyl,

3-acetamidopyrrolidin-1-ylcarbonyl, 3-t-butoxycarbonylaminopyrrolidin-1-ylcarbonyl,

N-methyl-N-(N-methylpyrrolidin-3-yl)aminocarbonyl, N-methylhomopiperazin-4-ylcarbonyl,

1-hydroxy-3-methylbut-2-ylaminocarbonyl, 1-hydroxyprop-2-ylaminocarbonyl,

2,6-dimethylmorpholinocarbonyl, N-methyl-N-(2-methoxyethyl)aminocarbonyl,

N-methyl-N-(N-benzylpyrrolidin-3-yl)aminocarbonyl, 2-oxohomopiperazin-4-ylcarbonyl,

3-methylpyrrolidin-1-ylcarbonyl, 2-(N,N-dimethylaminomethyl)piperidin-1-yl,

N-acetylhomopiperazin-4-ylcarbonyl, N-methyl-N-(2,3-dihydroxypropyl)aminocarbonyl,

homopiperazin-1-ylcarbonyl, 2-(N-methylamino)ethylaminocarbonyl,

2-hydroxymethylpyrrolidin-1-ylcarbonyl, 3-N,N-dimethylaminopyrrolidin-1-ylcarbonyl,

N-methyl-N-(3-N,N-dimethylaminoethyl)aminocarbonyl, 2-4dioxoimidazolidin-5-ylacetyl,

2-oxopyrrolidin-5-ylcarbonyl, pyrazin-2-ylcarbonyl, N,N-dimethylaminoacetyl,

2-acetamidopropionyl, 3-(N,N-dimethylcarbamoyl)propionyl, mesylacetyl,

N-methyl-1,2,5,6-pyridylcarbonyl, 5-methylisoxazol-4-ylcarbonyl, 3-hydroxypropionyl,

3,5-dimethyl-1,2,4triazol-1-ylacetyl, tetrahydrofuran-3-yloxyacetyl,

4-oxo-1,4-dihydropyrid-1-ylacetyl, 3-pyrazin-2-ylpropionyl,

4-carboxypiperidin-1-ylcarbonyl, piperidin-4-ylcarbonyl, piperidin-3-ylcarbonyl,

1-t-butoxycarbonylpyrrolidin-3-ylcarboxyl and pyrrolidin-3-ylcarbonyl.

In another further aspect of the invention preferably R⁵ is —C(O)NR⁹R¹⁰, —C(O)R⁹ or —C(O)C(O)R⁹; wherein

R⁹ and R¹⁰ are independently hydrogen, C₁₋₁₀alkyl, C₁₋₄alkoxy, carbocyclyl or heterocyclyl wherein R⁹ and R¹⁰ independently may be optionally substituted on carbon by one or more R¹¹; and wherein if said heterocyclyl contains an —NH— moiety that nitrogen may be optionally substituted by R¹²;

or R⁹ and R¹⁰ together with the nitrogen to which they are attached form a heterocyclic ring optionally substituted on carbon by one or more R¹³; and wherein if said heterocyclic ring contains an —NH— moiety that nitrogen may be optionally substituted by R¹⁴;

R¹¹ and R¹³ are independently selected from halo, hydroxy, carbamoyl, amino, carboxy, carbamoyl, C₁₋₄alkyl, C₁₋₄alkoxy, C₁₋₄alkanoylamino, C₁₋₄alkoxycarbonyl, N-(C₁₋₄alkyl)amino, N,N-(C₁₋₄alkyl)₂amino, C₁₋₄alkoxycarbonylamino, N-(C₁₋₄alkyl)carbamoyl, N,N-(C₁₋₄alkyl)₂carbamoyl, C₁₋₄alkylS(O)ₐ wherein a is 0 or 2, heterocyclyl, heterocyclyloxy or carbocyclyl; wherein R¹¹ and R¹³ independently may be optionally substituted on carbon by one or more R¹⁵;

R¹² and R¹⁴ are independently selected from C₁₋₄alkyl, C₁₋₄alkanoyl, C₁₋₄alkoxycarbonyl, carbamoyl, N-(C₁₋₄alkyl)carbamoyl, N,N-(C₁₋₄alkyl)₂carbamoyl, heterocyclyl and carbocyclylC₁₋₄alkyl;

R¹⁵ is selected from hydroxy, amino, C₁₋₄alkoxycarbonylamino, C₁₋₄alkoxy, N,N-(C₁₋₄alkyl)₂amino, heterocyclyl.

In another further aspect of the invention more preferably R⁵ is —C(O)NR⁹R¹⁰, —C(O)R⁹ or —C(O)C(O)R⁹; wherein

R⁹ and R¹⁰ are independently hydrogen, C₁₋₆alkyl, C₁₋₄alkoxy, cyclopropyl, 2-H-5,6-dihydropyranyl, 4H-5,6-dihydropyranyl, 2-oxotetrahydrofuranyl, tetrahdrofuranyl, pyrrolidinyl, 2-oxopyrrolidinyl, pyrazinyl, 1,2,5,6-tetrahydropyridinyl, isoxazolyl, 1,2,4-triazolyl, tetrahydrothienyl, tetrahydropyranyl or piperidinyl; wherein R⁹ and R¹⁰ independently may be optionally substituted on carbon by one or more R¹¹; and wherein if said heterocyclyl contains an —NH— moiety that nitrogen may be optionally substituted by R¹²;

or R⁹ and R¹⁰ together with the nitrogen to which they are attached form piperidinyl, pyrrolidinyl, homopiperazinyl, 4-oxohomopiperazinyl, morpholino, 2-oxopiperazinyl or piperazinyl optionally substituted on carbon by one or more R¹³; and wherein if said heterocyclic ring contains an —NH— moiety that nitrogen may be optionally substituted by R¹⁴ ;

R¹¹ and R¹³ are independently selected from halo, hydroxy, carboxy, carbamoyl, amino, C₁₋₄-alkyl, C₁₋₄alkoxy, C₁₋₄alkanoylamino, C₁₋₄alkoxycarbonyl, N-(C₁₋₄alkyl)amino, N,N-(C₁₋₄alkyl)₂amino, C₁₋₄alkoxycarbonylamino, N-(C₁₋₄alkyl)carbamoyl, N,N-(C₁₋₄alkyl)₂carbamoyl, C₁₋₄alkylS(O)ₐ wherein a is 0 or 2, 1,1-dioxotetrahydrothienyl, triazolyl, 2-pyrrolidinone, tetrahydropyran, pyridyl, piperidinyl, 2,4-dioxoimidazolidinyl, tetrahydrofuranyl, tetrahydrofuranyloxy, 4-oxo-1,4-dihydropyridinyl, pyrazinyl, cyclohexyl or phenyl; wherein R¹¹ and R¹³ independently may be optionally substituted on carbon by one or more R¹⁵;

R¹² and R¹⁴ are independently selected from C₁₋₄alkyl, C₁₋₄alkanoyl, carbamoyl, C₁₋₄alkoxycarbonyl, N-(C₁₋₄alkyl)carbamoyl, N,N-(C₁₋₄ alkyl)₂carbamoyl, pyridyl and benzyl;

R¹⁵ is selected from hydroxy, C₁₋₄alkoxy, amino, C₁₋₄alkoxycarbonylamino, N,N-(C₁₋₄alkyl)₂amino and pyrrolininyl.

In another further aspect of the invention, particularly R⁵ is

3-ethoxycarbonylpiperidin-1-ylcarbonyl, 3-hydroxymethylpiperidin-1-ylcarbonyl,

4-carbamoylpiperidin-1-ylcarbonyl, 4-ethoxycarbonylpiperidin-1-ylcarbonyl,

1-acetylpiperidin-4-ylcarbonyl, 1-N,N-dimethylcarbamoylpiperidin-3-ylcarbonyl,

1-acetylpiperidin-3-ylcarbonyl, 3-methoxycarbonylpyrrolidin-1-ylcarbonyl,

1-N-methylcarbamoylpiperidin-3-ylcarbonyl, 1-carbamoylpiperidin-3-ylcarbonyl,

3-N,N-dimethylcarbamoylpyrrolidin-1-ylcarbonyl, 1-carbamoylpiperidin-4-ylcarbonyl,

1-N,N-dimethylcarbamoylpiperidin-4-ylcarbonyl, 1-acetlypyrrolidin-3-ylcarbonyl,

1-N,N-dimethylcarbamoylpyrrolidin-3-ylcarbonyl,

1-N-methylcarbamoylpyrrolidin-3-ylcarbonyl, 1-hydroxy-3-methylpent-2-ylaminocarbonyl,

3-hydroxy-3-phenylprop-2-ylaminocarbonyl, (1-hydroxycyclohex-1-yl)methylaminocarbonyl,

N-methyl-N-(3-phenyl-3-hydroxyprop-2-yl)aminocarbonyl,

2-hydroxymethylpiperidin-1-ylcarbonyl, 2-hydroxymethylpyrrolidin-1-ylcarbonyl,

3-hydroxypiperidin-1-ylcarbonyl, 3-cyclohexyl-1-hydroxyprop-2-ylaminocarbonyl,

3-hydroxyprop-2-ylaminocarbonyl, 3-hydroxypyrrolidin-1-ylcarbonyl,

4-N,N-dimethylcarbamoylpiperidin-1-ylcarbonyl,

4-N-methylcarbamoylpiperidin-1-ylcarbonyl, 3-hydroxypent-3-ylcarbonylamino,

N,N-dimethylaminoacetyl, methoxyacetyl, ethoxyacetyl, 2-hydroxybut-2-ylcarbonyl,

N-methylpyrrolidin-2-ylcarbonyl, 2-oxotetrahydrofuran-5-ylcarbonyl,

tetrahydrofuran-2-ylcarbonyl, 2-hydroxy-1-acetamidoethyl, 3-methoxypropionyl,

2-methoxyethoxyacetyl, 3-ethoxypropionyl, 3-methylthiopropionyl,

2-trifluoromethyl-2-hydroxypropionyl, 2-phenyl-2-methoxypropionyl, piperidin-1-ylacetyl,

tetrahydrofuran-3-yloxyacetyl, tetrahydrothien-2-ylcarbonyl, 3-hydroxypropionyl,

(3-hydroxymethyltetrahydropyran-3-yl)carbonyl, (1-carbamoylcycloprop-1-yl)carbonyl,

3-acetamidopropionyl, 2-H-5,6-dihydropyran-3-ylcarbonyl, 3-ethylthioprop-2-ylcarbonyl,

2,3,4-H-dihydpyran-2-ylcarbonyl, 1-hydroxy-3-methylbutylcarbonyl,

3-N,N-dimethylaminopyrrolidin-1-ylcarbonyl, 2-methyl-2-hydroxypropionyl,

N-methyl-N-(2-hydroxyethyl)aminocarbonyl, N,N-di-(2-hydroxyethyl)aminocarbonyl,

tetrahydrofuran-2-ylmethylaminocarbonyl, 2,6-dimethylpiperazin-4-ylcarbonyl,

4-hydroxypiperidin-1-ylcarbonyl, N-methyl-N-(N-methylpiperidin-4-yl)aminocarbonyl,

2-methoxyprop-2-ylaminocarbonyl, 2-hydroxypropylaminocarbonyl,

2-methoxyethylaminocarbonyl, 2-methoxymethylpyrrolidin-1-ylcarbonyl,

2-(pyrrolidin-1-ylmethyl)pyrrolidin-1-ylcarbonyl, 3-hydroxypiperidin-1-ylcarbonyl,

N-methyl-N-(3-N,N-dimethylaminopropyl)aminocarbonyl,

N,N-di-(2-methoxyethyl)aminocarbonyl, 1-acetylpiperazintylcarbonyl,

3-acetamidopyrrolidin-1-ylcarbonyl, 3-t-butoxycarbonylaminopyrrolidin-1-ylcarbonyl,

N-methyl-N(N-methylpyrrolidin-3-yl)aminocarbonyl, N-methylhomopiperazin-4-ylcarbonyl,

1-hydroxy-3-methylbut-2-ylaminocarbonyl, 1-hydroxyprop-2-ylaminocarbonyl,

2,6-dimethylmorpholinocarbonyl, N-methyl-N-(2-methoxyethyl)aminocarbonyl,

N-methyl-N-(N-benzylpyrrolidin-3-yl)aminocarbonyl, 2-oxohomopiperazin-4-ylcarbonyl,

3-mesylpyrrolidin-1-ylcarbonyl, 2-(NN-dimethylaminomethyl)piperidin-1-yl,

N-acetylhomopiperazin-4-ylcarbonyl, N-methyl-N-(2,3-dihydroxypropyl)aminocarbonyl,

homopiperazin-1-ylcarbonyl, 2-(N-methylamino)ethylaminocarbonyl,

2-hydroxymethylpyrrolidin-1-ylcarbonyl, 3-N,N-dimethylaminopyrrolidin-1-ylcarbonyl,

N-methyl-N-(3-N,N-dimethylaminoethyl)aminocarbonyl, 2,4-dioxoimidazolidin-5-ylacetyl,

2-oxopyrrolidin-5-ylcarbonyl, pyrazin-2-ylcarbonyl, N,N-dimethylaminoacetyl,

2-acetamidopropionyl, 3-(N,N-dimethylcarbamoyl)propionyl, mesylacetyl,

N-methyl-1,2,5,6-pyridylcarbonyl, 5-methylisoxazol-4-ylcarbonyl, 3-hydroxypropionyl,

3,5-dimethyl-1,2,4-triazol-1-ylacetyl, tetrahydrofuran-3-yloxyacetyl,

4-oxo-1,4-dihydropyrid-1-ylacetyl, 3-pyrazin-2-ylpropionyl,

4-carboxypiperidin-1-ylcarbonyl, piperidin-4-ylcarbonyl, piperidin-3-ylcarbonyl,

1-t-butoxycarbonylpyrrolidin-3-ylcarboyl, pyrrolidin-3-ylcarbonyl, t-butoxycarbonyl,

morpholinocarbonyl, tetrahydrofuran-3-ylcarbonyl, 2-(1,1-dioxotetrahydrothien-3-yl)acetyl,

3-carbamoylpiperidin-1-ylcarbonyl, 2,2,2-trichloroacetyl,

4-(pyrid-2-yl)piperazin-1-ylcarbonyl, 3-(pyrid-4-yl)propionyl,

N-[2-(pyrid-4-yl)ethyl]-N-methylaminocarbonyl,

N-(pyrid-3-ylmethyl)-N-methylaminocarbonyl,

N-(1-methylpiperidyl)-N-methylaminocarbonyl, 4-hydroxymethylpiperid-1-ylcarbonyl,

4-(pyrid-4-yl)piperid-1-ylcarbonyl, 4-hydroxyethylpiperid-1-ylcarbonyl,

3-methoxy-2-(t-butoxycarbonylamino)propionyl, 2-(1,2,4-triazol-1-yl)acetyl,

2-(2-pyrrolidinon-1-yl)acetyl, tetrahydropyranylcarbonyl,

2-[3-(t-butoxycarbonylamino)cyclohexyl]acetyl, 3-methoxy-2-aminopropionyl,

2-(3-aminocyclohexyl)acetyl, 4-pyrid-4-ylpiperidin-1-yloxalyl,

4-pyrid-2-ylpiperazin-1-yloxalyl, 4-hydroxypiperidin-1-yl, 2-hydroxyethylaminocarbonyl and N-methoxy-N-methylaminocarbonyl.

In an additional further aspect of the invention, particularly R⁵ is

3-ethoxycarbonylpiperidin-1-ylcarbonyl, 3-hydroxymethylpiperidin-1-ylcarbonyl,

4-carbamoylpiperidin-1-ylcarbonyl, 4-ethoxycarbonylpiperidin-1-ylcarbonyl,

1-acetylpiperidin-4-ylcarbonyl, 1-N,N-dimethylcarbamoylpiperidin-3-ylcarbonyl,

1-acetylpiperidin-3-ylcarbonyl, 3-methoxycarbonylpyrrolidin-1-ylcarbonyl,

1-N-methylcarbamoylpiperidin-3-ylcarbonyl, 1-carbamoylpiperidin-3-ylcarbonyl,

3-N,N-dimethylcarbamoylpyrrolidin-1-ylcarbonyl, 1-carbamoylpiperidin-4-ylcarbonyl,

1-N,N-dimethylcarbamoylpiperidin-4-ylcarbonyl, 1-acetlypyrrolidin-3-ylcarbonyl,

1-N,N-dimethylcarbamoylpyrrolidin-3-ylcarbonyl,

1-N-methylcarbamoylpyrrolidin-3-ylcarbonyl, 1-hydroxy-3-methylpent-2-ylaminocarbonyl,

3-hydroxy-3-phenylprop-2-ylaminocarbonyl, (1-hydroxycyclohex-1-yl)methylaminocarbonyl,

N-methyl-N-(3-phenyl-3-hydroxyprop-2-yl)aminocarbonyl,

2-hydroxymethylpiperidin-1-ylcarbonyl, 2-hydroxymethylpyrrolidin-1-ylcarbonyl,

3-hydroxypiperidin-1-ylcarbonyl, 3-cyclohexyl-1-hydroxyprop-2-ylaminocarbonyl,

3-hydroxyprop-2-ylaminocarbonyl, 3-hydroxypyrrolidin-1-ylcarbonyl,

4-N,N-dimethylcarbamoylpiperidin-1-ylcarbonyl,

4-N-methylcarbamoylpiperidin-1-ylcarbonyl, 3-hydroxypent-3-ylcarbonylamino,

N,N-dimethylaminoacetyl, methoxyacetyl, ethoxyacetyl, 2-hydroxybut-2-ylcarbonyl,

N-methylpyrrolidin-2-ylcarbonyl, 2-oxotetrahydrofuran-5-ylcarbonyl,

tetrahydrofuran-2-ylcarbonyl, 2-hydroxy-1-acetalamidoethyl, 3-methoxypropionyl,

2-methoxyethoxyacetyl, 3-ethoxypropionyl, 3-methylthiopropionyl,

2-trifluoromethyl-2-hydroxypropionyl, 2-phenyl-2-methoxypropionyl, piperidin-1-ylacetyl,

tetrahydrofuran-3-yloxyacetyl, tetrahydrothien-2-ylcarbonyl, 3-hydroxypropionyl,

(3-hydroxymethyltetrahydropyran-3-yl)carbonyl, (1-carbamoylcycloprop-1-yl)carbonyl,

3-acetamidopropionyl, 2-H-5,6-dihydropyran-3-ylcarbonyl, 3-ethylthioprop-2-ylcarbonyl,

2,3-4-H-dihydpyran-2-ylcarbonyl, 1-hydroxy-3-methylbutylcarbonyl,

3-N,N-dimethylaminopyrrolidin-1-ylcarbonyl, 2-methyl-2-hydroxypropionyl,

N-methyl-N-(2-hydroxyethyl)aminocarbonyl, N,N-(2-hydroxyethyl)aminocarbonyl,

tetrahydrofuran-2-ylmethylaminocarbonyl, 2,6-dimethylpiperazin-4-ylcarbonyl,

4-hydroxypiperidin-1-ylcarbonyl, N-methyl-N-(N-methylpiperidinyl)aminocarbonyl,

2-methoxyprop-2-ylaminocarbonyl, 2-hydroxypropylaminocarbonyl,

2-methoxyethylaminocarbonyl, 2-methoxymethylpyrrolidin-1-ylcarbonyl,

2-(pyrrolidin-1-ylmethyl)pyrrolidin-1-ylcarbonyl, 3-hydroxypiperidin-1-ylcarbonyl,

N-methyl-N-(3-N,N-dimethylaminopropyl)aminocarbonyl,

N,N-di-(2-methoxyethyl)aminocarbonyl, 1-acetylpiperazin-4-ylcarbonyl,

3-acetamidopyrrolidin-1-ylcarbonyl, 3-t-butoxycarbonylaminopyrrolidin-1-ylcarbonyl,

N-methyl-N-(N-methylpyrrolidin-3-yl)aminocarbonyl, N-methylhomopiperazin-4-ylcarbonyl,

1-hydroxy-3-methylbut-2-ylaminocarbonyl, 1-hydroxyprop-2-ylaminocarbonyl,

2,6-dimethylmorpholinocarbonyl, N-methyl-N-(2-methoxyethyl)aminocarbonyl,

N-methyl-N-(N-benzylpyrrolidin-3-yl)aminocarbonyl, 2-oxohomopiperazin-4-ylcarbonyl,

3-mesylpyrrolidin-1-ylcarbonyl, 2-(N,N-dimethylaminomethyl)piperidin-1-yl,

N-acetylhomopiperazin-4-ylcarbonyl, N-methyl-N-(2,3-dihydroxypropyl)aminocarbonyl,

homopiperazin-1-ylcarbonyl, 2-(N-methylamino)ethylaminocarbonyl,

2-hydroxymethylpyrrolidin-1-ylcarbonyl, 3-N,N-dimethylaminopyrrolidin-1-ylcarbonyl,

N-methyl-N-(3-N,N-dimethylaminoethyl)aminocarbonyl, 2,4-dioxoimidazolidin-5-ylacetyl,

2-oxopyrrolidin-5-ylcarbonyl, pyrazin-2-ylcarbonyl, N,N-dimethylaminoacetyl,

2-acetamidopropionyl, 3-(N,N-dimethylcarbamoyl)propionyl, mesylacetyl,

N-methyl-1,2,5,6-pyridylcarbonyl, 5-methylisoxazolylcarbonyl, 3-hydroxypropionyl,

3,5-dimethyl-1,2,4-triazol-1-ylacetyl, tetrahydrofuran-3-yloxyacetyl,

4-oxo-1,4-dihydropyrid-1-ylacetyl, 3-pyrazin-2-ylpropionyl,

4-carboxypiperidin-1-ylcarbonyl, piperidin-4-ylcarbonyl, piperidin-3-ylcarbonyl,

1-t-butoxycarbonylpyrrolidin-3-ylcarboyl, pyrrolidin-3-ylcarbonyl, t-butoxycarbonyl,

morpholinocarbonyl, tetrahydrofuran-3-ylcarbonyl, 2-(1,1-dioxotetrahydrothien-3-yl)acetyl,

3-carbamoylpiperidin-1-ylcarbonyl, 2,2,2-trichloroacetyl,

4-(pyrid-2-yl)piperazin-1-ylcarbonyl, 3-(pyrid-4-yl)propionyl,

N-[2-(pyrid-4-yl)ethyl]-N-methylaminocarbonyl,

N-(pyrid-3-ylmethyl)-N-methylaminocarbonyl,

N-(1-methylpiperidyl)-N-methylaminocarbonyl, 4-hydroxymethylpiperid-1-ylcarbonyl,

4-(pyrid-4-yl)piperid-1-ylcarbonyl, 4-hydroxyethylpiperid-1-ylcarbonyl,

3-methoxy-2-(t-butoxycarbonylamino)propionyl, 2-(1,2,4-triazol-1-yl)acetyl,

2-(2-pyrrolidinon-1-yl)acetyl, tetrahydropyran-4-ylcarbonyl,

2-[3-(t-butoxycarbonylamino)cyclohexyl]acetyl, 3-methoxy-2-aminopropionyl,

2-(3-aminocyclohexyl)acetyl, 4-pyridylpiperidin-1-yloxalyl,

4pyrid-2-ylpiperazin-1-yloxalyl, 4-hydroxypiperidin-1-yl, 2-hydroxyethylaminocarbonyl,

N-methoxy-N-methylaminocarbonyl, 3-N,N-dimethylcarbamoylpiperidin-1-ylcarbonyl,

3-N-methylcarbamoylpiperidin-1-ylcarbonyl and 3-carboxypiperidin-1-ylcarbonyl.

Preferably R⁶ and R⁸ are independently selected from halo or C₁₋₄alkyl.

More preferably R⁶ and R⁸ are independently selected from fluoro, chloro, bromo or methyl.

Preferably R⁶ is 2-methyl.

Preferably R⁶ is 2-methyl and R⁴ is methyl.

In one aspect of the invention, preferably m is 0.

In an further aspect of the invention, preferably m is 1.

Preferably m is 0 or 1.

Where m is 1, preferably the substituent R⁶ is in the 2-position, i.e. ortho to the —NHR⁵ substituent.

Therefore in an preferred aspect of the invention, there is provided a compound of formula (I) (as depicted above), wherein:

R¹ is selected from C₁₋₄alkyl optionally substituted on carbon by one or more R⁷ wherein R⁷ is C₁₋₄alkoxy;

R² and R³ together form —(CH₂)₄— or —(CH)₄— optionally substituted by R⁸; wherein R⁸ is selected from halo or C₁₋₄alkyl;

R² and R³ together form —(CH₂)₄— or —(CH)₄— optionally substituted by R⁸; wherein R⁸ is selected from fluoro, bromo or methyl;

R⁴ is methyl or isopropyl;

R⁵ is —C(O)NR⁹R¹⁰, —C(O)R⁹ or —C(O)C(O)R⁹; wherein

R⁹ and R¹⁰ are independently C₁₋₁₀alkyl, C₁₋₄alkoxy or heterocyclyl wherein R⁹ and R¹⁰ independently may be optionally substituted on carbon by one or more R¹¹; and wherein if said heterocyclyl contains an —NH— moiety that nitrogen may be optionally substituted by R¹²;

or R⁹ and R¹⁰ together with the nitrogen to which they are attached form a heterocyclic ring optionally substituted on carbon by one or more R¹³; and wherein if said heterocyclic ring contains an —NH— moiety that nitrogen may be optionally substituted by R¹⁴;

R¹¹ and R¹³ are independently selected from halo, hydroxy, carbamoyl, amino, C₁₋₄alkyl, C₁₋₄alkoxy, C₁₋₄alkoxycarbonylamino, heterocyclyl, carbocyclyl; wherein R¹¹ and R¹³ independently may be optionally substituted on carbon by one or more R¹⁵;

R¹² and R¹⁴ are independently selected from C₁₋₄alkyl, heterocyclyl;

R¹⁵ is selected from hydroxy, amino, C₁₋₄alkoxycarbonylamino;

R⁶ and R⁸ are independently selected from halo or C₁₋₄alkyl.

or a pharmaceutically acceptable salt, prodrug or solvate thereof.

Therefore in an preferred aspect of the invention, there is provided a compound of formula (I) (as depicted above), wherein:

R¹ is selected from C₁₋₄alkyl optionally substituted on carbon by one or more R⁷ wherein R⁷ is C₁₋₄alkoxy;

R² and R³ together form —(CH₂)₄— or —(CH)₄— optionally substituted by R⁸; wherein R⁸ is selected from fluoro, bromo or methyl;

R⁴ is methyl or isopropyl;

R⁵ is —C(O)NR⁹R¹⁰, —C(O)R⁹ or —C(O)C(O)R⁹; wherein

R⁹ and R¹⁰ are independently C₁₋₁₀alkyl, C₁₋₄alkoxy or heterocyclyl wherein R⁹ and R¹⁰ independently may be optionally substituted on carbon by one or more R¹¹; and wherein if said heterocyclyl contains an —NH— moiety that nitrogen may be optionally substituted by R¹²;

or R⁹ and R¹⁰ together with the nitrogen to which they are attached form a heterocyclic ring optionally substituted on carbon by one or more R¹³; and wherein if said heterocyclic ring contains an —NH— moiety that nitrogen may be optionally substituted by R¹⁴;

R¹¹ and R¹³ are independently selected from halo, hydroxy, carbamoyl, amino, C₁₋₄alkyl, C₁₋₄alkoxy, C₁₋₄alkoxycarbonylamino, heterocyclyl, carbocyclyl; wherein R¹¹ and R¹³ independently may be optionally substituted on carbon by one or more R¹⁵;

R¹² and R¹⁴ are independently selected from C₁₋₄alkyl, heterocyclyl;

R¹⁵ is selected from hydroxy, amino, C₁₋₄alkoxycarbonylamino;

R⁶ and R⁸ are independently selected from halo or C₁₋₄alkyl;

m is 0 or 1;

or a pharmaceutically acceptable salt, prodrug or solvate thereof.

Therefore in a further preferred aspect of the invention, there is provided a compound of formula (I) (as depicted above), wherein:

R¹ is selected from C₁₋₄alkyl optionally substituted on carbon by one or more R⁷ wherein R⁷ is C₁₋₄alkoxy;

R² and R³ together form —(CH₂)₄— or —(CH)₄— optionally substituted by R⁸; wherein R⁸ is selected from halo or C₁₋₄alkyl;

R⁴ is methyl or isopropyl;

R⁵ is —C(O)NR⁹R¹⁰, —C(O)R⁹ or —C(O)C(O)R⁹; wherein

R⁹ and R¹⁰ are independently hydrogen, C₁₋₁₀alkyl, C₁₋₄alkoxy, carbocyclyl or heterocyclyl wherein R⁹ and R¹⁰ independently may be optionally substituted on carbon by one or more R¹¹ ; and wherein if said heterocyclyl contains an —NH— moiety that nitrogen may be optionally substituted by R¹²;

or R⁹ and R¹⁰ together with the nitrogen to which they are attached form a heterocyclic ring optionally substituted on carbon by one or more R¹³; and wherein if said heterocyclic ring contains an —NH— moiety that nitrogen may be optionally substituted by R¹⁴;

R¹¹ and R¹³ are independently selected from halo, hydroxy, carbamoyl, amino, carboxy, carbamoyl, C₁₋₄alkyl, C₁₋₄alkoxy, C₁₋₄alkanoylamino, C₁₋₄alkoxycarbomyl, N-(C₁₋₄alkyl)amino, N,N-(C₁₋₄alkyl)₂amino, C₁₋₄alkoxycarbonylamino, N-(C₁₋₄alkyl)carbamoyl, N,N-(C₁₋₄alkyl)₂carbamoyl, C₁₋₄alkylS(O)ₐ wherein a is 0 or 2, heterocyclyl, heterocyclyloxy or carbocyclyl; wherein R¹¹ and R¹³ independently may be optionally substituted on carbon by one or more R¹⁵;

R¹² and R¹⁴ are independently selected from C₁₋₄alkyl, C₁₋₄alkanoyl, C₁₋₄alkoxycarbonyl, carbamoyl, N-(C₁₋₄alkyl)carbamoyl, N,N-(C₁₋₄alkyl)carbamoyl, heterocyclyl and carbocyclylC₁₋₄alkyl;

R¹⁵ is selected from hydroxy, amino, C₁₋₄alkoxycarbonylamino, C₁₋₄alkoxy, N,N-(C₁₋₄alkyl)₂amino, heterocyclyl; and

R⁶ is 2-methyl;

m is 0 or 1;

or a pharmaceutically acceptable salt, prodrug or solvate thereof.

In another aspect of the invention, preferred compounds of the invention are any one of the Examples or a pharmaceutically acceptable salt, prodrug or solvate thereof.

In an additional aspect of the invention, there is provided a compound of formula (I). selected from:

3-(morpholinocarbonylamino)-4-methyl-9-isopropyl-9H-carbazole;

3-(3-carbamoylpiperidin-1-ylcarbonylamino)-2,4-dimethyl-9-isopropyl-9H-carbazole;

3-(1,1-dioxotetrahydrothien-3-ylmethylcarbonylamino)-4-methyl-9-isopropyl-9H-carbazole;

3-(morpholinocarbonylamino)-4-methyl-6-fluoro-9-isopropyl-9H-carbazole;

3-(3-carbamoylpiperidin-1-ylcarbonylamino)-4-methyl-9-isopropyl-9H-carbazole;

3-(4-hydroxypiperidin-1-ylcarbonylamino)-4-methyl-6-fluoro-9-isopropyl-9H-carbazole;

3-[3-(N-methylcarbamoyl)piperidin-1-ylcarbonylamino]-2,4-dimethyl-9-isopropyl-9H-carbazole;

3-[1-(N,N-ethylcarbamoyl)piperidin-4-ylcarbonylamino]-2,4-diethyl-9-isopropyl-9H-carbazole;

3-[1-(N,N-dimethylcarbamoyl)pyrrolidin-3-ylcarbonylamino]-2,4-dimethyl-9-isopropyl-9H-carbazole; and

3-[4-hydroxypiperidin-1-ylcarbonylamino]-2-methyl-9-isopropyl-9H-carbazole;

or a pharmaceutically acceptable salt, prodrug or solvate thereof.

Preferred aspects of the invention are those which relate to the compound of formula (I) or a pharmaceutically acceptable salt thereof.

Another aspect of the present invention provides a process for preparing a compound of formula (I) or a pharmaceutically acceptable salt or prodrug or solvate thereof which process (wherein variable groups are, unless otherwise specified, as defined in formula (I)) comprises of:

Process a): for compounds of formula (I) wherein R⁵ is —C(O)R⁹; reacting an amine of formula (II):

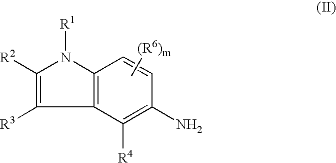

with an acid of formula (III):

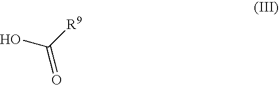

or an activated derivative thereof; or

Process b): for compounds of formula (I) wherein R⁵ is —C(O)NR⁹R¹⁰; by reacting a compound of formula (IV):

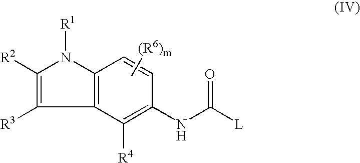

wherein L is a displaceable group; with an amine of formula (V):

HNR⁹R¹⁰ (V)

Process c): for compounds of formula (I) wherein R⁵ is —C(O)NR⁹R¹⁰; reacting a compound of formula (I) with a compound of formula (VI):

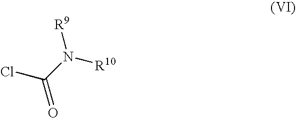

Process d): for compounds of formula (I) wherein R⁵ is —C(O)NR⁹R¹⁰ and one of R⁹ and R¹⁰ is hydrogen; reacting a compound of formula (II) with an isocyanate of formula (VII):

O═—N—R^{a} (VII)

wherein R^{a} is R⁹ or R¹⁰ not equal to hydrogen;

Process e): reacting a compound of formula (VIII):

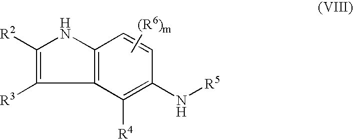

with a compound of formula (IX):

R¹—Z (IX)

wherein Z is a displaceable group or when R¹ is C₁₋₄alkanoyl Z may be hydroxy;

Process f): for compounds of formula (I) wherein R⁵ is —C(O)NR⁹R¹⁰; by reacting a compound of formula (X):

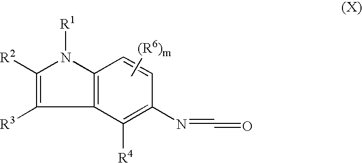

with an amine of formula (V);

and thereafter if necessary:

i) converting a compound of the formula (I) into another compound of the formula (I);

ii) removing any protecting groups;

iii) forming a pharmaceutically acceptable salt or prodrug or solvate thereof.

L is a displaceable group. Suitable values for L are phenols for example p-nitrophenol or penta-fluorophenol.

Y is a displaceable group. A suitable values for Y is halo, for example chloro, bromo or trifluoromethylsulphonyloxy.

Specific reaction conditions for the above reactions are as follows.

Process a)

Amines of formula (II) and acids of formula (III) may be coupled together in the presence of a suitable coupling reagent. Standard peptide coupling reagents known in the art can be employed as suitable coupling reagents, or for example carbonyldiimidazole and dicyclohexyl-carbodiimide, optionally in the presence of a catalyst such as dimethylaminopyridine or 4-pyrrolidinopyridine, optionally in the presence of a base for example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

The amines of formula (II) and acids of formula (III) are commercially available or they are known compounds or they are prepared by processes known in the art, see for example those processes described in the examples for preparation of compounds of formula

In addition, compounds of formula (II) may be prepared by the following schemes:

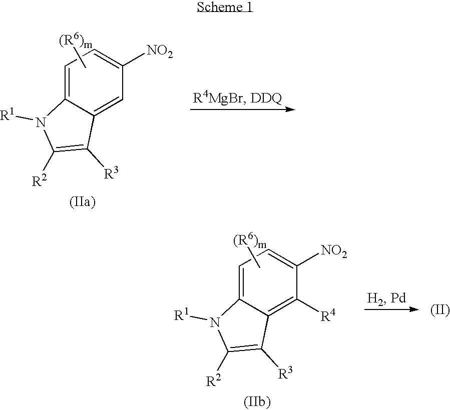

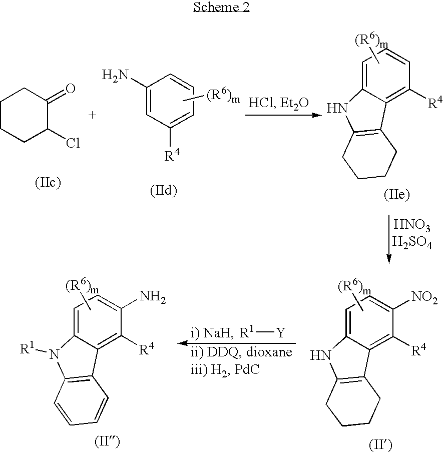

Wherein Y is a leaving group as defined above and wherein step ii) of the final step in Scheme 2 is omitted if compounds of formula (II) wherein R² and R³ together form —(CH₂)₄— are required.

The skilled reader will appreciate that Scheme 2 could be adapted by starting with a substituted cyclohexane i.e. to give R⁸ and also that 2-oxo-3-chlorobutyl could be used as a starting material to give a compound of formula (II) wherein R² and R³ are both methyl. Furthermore in Scheme 2 the compound (IId) could have R⁴ equal to hydrogen and then the R⁴ group could be introduced at a later stage in the synthesis, for example by the route illustrated in Scheme 1.

For compounds of formula (II) wherein R⁶ is 1-methyl and R⁴ is methyl

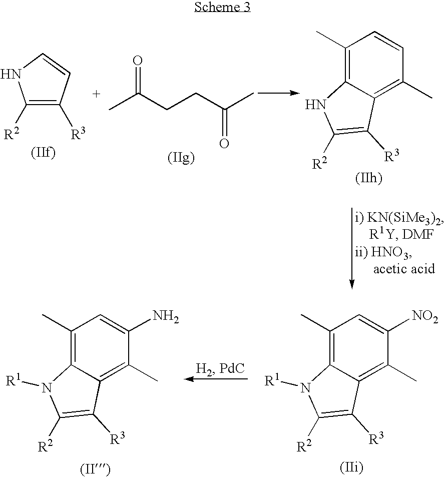

Wherein Y is a leaving group as defined above.

The skilled reader will note that Scheme 3 could be adapted to give other values of R⁴ and R⁶, but unless R⁶ is in the 1-position and equal to R⁴, two possible products would result from the first step in the synthesis that would need to be separated.

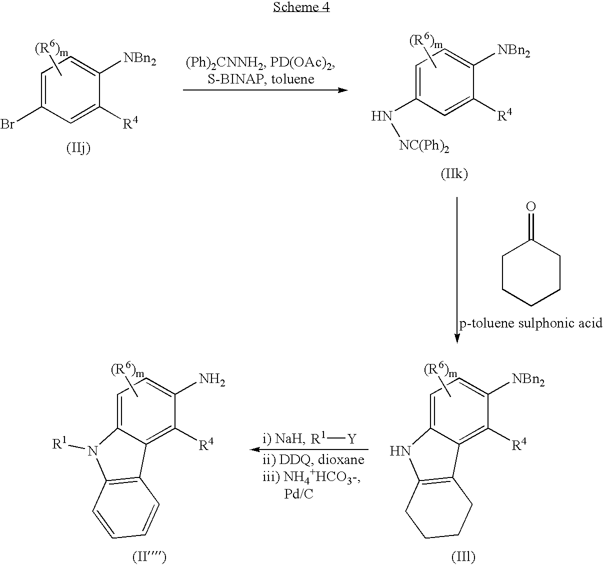

Wherein Y is a leaving group as defined above and wherein step ii) of the final step in Scheme 4 is omitted if compounds of formula (II) wherein R² and R³ together form —(CH₂)₄— are required.

The skilled reader will appreciate that Scheme 4 could be adapted by starting with a substituted cyclohexane i.e. to give R⁸ and also that 2-oxobutyl could be used as a starting material to give a compound of formula (II) wherein R² and R³ are both methyl.

Compounds of formula (IIa), (IIc), (IId), (IId), (IIg) and (IIj) are commercially available or they are known compounds or they are prepared by processes known in the art.

Process b)

Compounds of formula (IV) and amines of formula (V) may be reacted together in the presence of a suitable base, for example triethylamine, pyridine, or 2,6-di-alkyl-pyridines such as 2,6-lutidine or 2,6-di-tert-butylpyridine, or excess (V), in a suitable solvent such as dichloromethane, ethyl acetate or tetrahydrofuran. The reaction may conveniently be performed at a temperature in the range of −40 to 50° C.

The compounds of formula (IV) may be prepared from amines of formula (II) by standard processes known in the art. Compounds of formula (V) are commercially available or they are known compounds or they are prepared by processes known in the art.

Process c)

Compounds of formula (II) and compounds of formula (VI) may be reacted in the presence of a base, such as those described above, in a suitable solvent, such as dichloromethane, toluene or tetrahydrofuran. The reaction may conveniently be performed at a temperature in the range of −40 to 100° C.

Compounds of formula (VI) are commercially available or they are known compounds or they are prepared by processes known in the art.

Process d)

Compounds of formula (II) and compounds of formula (VII) may be reacted in the presence of a suitable solvent, such as toluene, dichloromethane or tetrahydrofuran.

Compounds of formula (VII) are commercially available or they are known compounds or they are prepared by processes known in the art.

Process e)

Compounds of formula (VIII) and compounds of formula (IX) may be reacted together in the presence of a suitable base, for example sodium hydride, potassium hexamethyldisilazane, triethylamine, pyridine, or 2,6-di-alkyl-pyridines such as 2,6-lutidine or 2,6-di-tert-butylpyridine, in a suitable solvent such as dichloromethane, ethyl acetate or tetrahydrofuran. The reaction may conveniently be performed at a temperature in the range of 25° C. to reflux. When R¹ is C₁₋₄alkanoyl and Z is hydroxy compounds of formula (VIII) and compounds of formula (IX) may be reacted together in conditions such as those described in Process a) above.

Compounds of formula (VIII) may be prepared by the procedures described for the preparation of compounds of formula (I), but wherein R¹ is hydrogen. The person skilled in the art may also be aware if a protecting group for this nitrogen would be necessary. Therefore compounds of formula (VIII) may also be prepared by deprotecting a compound of formula (I) wherein R¹ is a nitrogen protecting group.

Compounds of formula (IX) are commercially available or they are known compounds or they are prepared by processes known in the art.

Process f)

Compounds of formula (X) and amines of formula (V) may be reacted together under similar conditions to those described in Process d).

Compounds of formula (X) may be prepared according to Scheme 5:

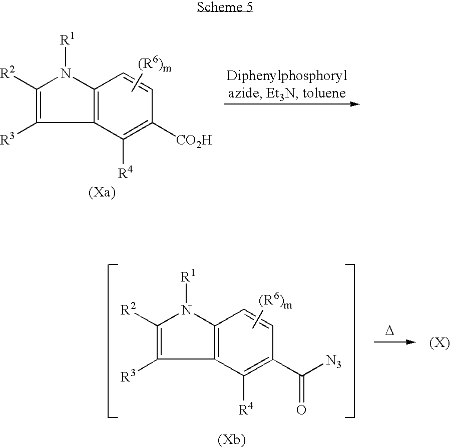

Compounds of formula (Xa) are commercially available or they are known compounds or they are prepared by processes known in the art, see for example the processes described in the examples.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halo group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl. The reader is referred to Advanced Organic Chemistry, 4^{th} Edition, by Jerry March, published by John Wiley & Sons 1992, for general guidance on reaction conditions and reagents.

It will also be appreciated that in some of the reactions mentioned herein it may be necessary/desirable to protect any sensitive groups in the compounds. The instances where protection is necessary or desirable and suitable Methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Biological Assays

The activity of compounds of the invention was measured in a neuropeptide Y5 receptor binding assay as follows. Compounds were also tested in binding assays for the neuropeptide Y₁ and neuropeptide Y₂ receptors. Activity against these 2 receptors is contraindicated for a neuropeptide Y5 antagonist.

a) Expression of Human Neuropeptide Y5 Receptor in High 5™ Insect Cells.

High 5™ insect cells were obtained from Invitrogen (catalogue N^{o} B855-02) and stored in liquid nitrogen. Cells were revived from liquid nitrogen storage and grown at 28° C. in 100 ml ExCell 405 (JRH Biosciences) serum free medium in a 250 ml conical flask (Corning) agitated at 140 rpm in an Innova 4330 orbital shaker (New Brunswick Scientific). Cultures were routinely sub-cultured every 3-4 days.

High 5™ insect cells were transfected with the human NPY5 receptor as follows. PCR primers were designed against the huNPY5 receptor sequence, Genbank Accession Number U56079 [Gerald et. al. (1996) Nature 382, 168-171], but starting at base 56 through to base 1393, to express the protein 10 amino acid residues shorter at the amino terminal end [see Borowsky et. al. (1998) Regulatory Peptides 75-76, 45-53]. These primers were used to amplify the huNPY5 receptor from human placenta genomic DNA by PCR. This was then sub-cloned into pZERO2 (obtained from Invitrogen) for sequencing and re-cloned into pFASTBAC1 (obtained from GIBCO BRL Life Technologies) for expression. Human NPYr was isolated from pZERO2 on BamHI fragment and sub-cloned into pFastbac1 on BamHI restriction site. The junctions were sequenced to ensure correct prior to expression.

A baculovirus containing the pFASTBAC1 was then generated using the Bac-to-Bac™ baculovirus expression system [Anderson et al (1996) FASEB Journal 10(6), 727-726] (obtained from GIBCO BRL Life Technologies) following the protocol supplied with this expression system by GIBCO BRL Life Technologies.

High 5™ insect cells were infected with the baculovirus to transfect the cells with the human neuropeptide Y5 receptor as follows: Batches were grown for membrane preparation by inoculating 5 L of ExCell 4051υ medium in a 7 L Bioreactor (FT-Applikon), with 1.75×10⁹ mid log High 5™ cells. After 2-3 days growth at 28° C. the mid log culture was infected with Baculovirus expressing the human NPY5 receptor at a multiplicity of infection (MOI) of 1.0. Cells (typically 1×10¹⁰) were harvested 48 hours post infection by centrifugation (Heraeus Omnifuge 2.0RS 30 min, 296 g, 4° C.) and flash frozen in liquid nitrogen for storage at −80° C.

b) Membrane Preparation Procedure

The following buffer was prepared daily and stored at 4° C. 50 mM Tris HCl pH 7.4, 5 mM EDTA and 10% w.w. sucrose. A protease inhibitor cocktail (Boehringer Mannheim) was added to both buffers according to the manufacturers instruction. Cells were thawed rapidly in three times their packed cell volume of hypotonic buffer (3:1 mix of water and buffer) and lysed routinely on ice using five Vibra Cell Sonicator (Sonics and Materials Inc.) bursts of ten seconds for the High 5™ insect cells. The cell lysate (typically 10-15 ml) was carefully loaded onto a 10 ml 41% sucrose cushion which was topped off with lysis buffer and spun at 150,000 g for 1 hour at 4° C. in a Beckman Optima LE-80K Ultracentrifuge. The membrane fraction was carefully removed from the inter-phase and diluted at least four fold with lysis buffer. The membrane pellets were recovered by centrifugation at 150,000 g for 20 min at 4° C. in a Beckman Optima LE-80K Ultracentrifuge and re-suspended at 5×10⁷ cell equivalents per ml. The re-suspended membranes were divided into working aliquots, routinely 1 ml, flash frozen in liquid nitrogen and stored frozen at −80° C. until use.

Prior to use the 1 ml High 5™ membranes were thawed and resuspended in 8 ml binding buffer (see below). Membranes are used at approximately 7 μg/ml of protein per incubate.

c) Neuropeptide Y5 Receptor Binding Assay

The following reagents were used:

Binding buffer: 50 mM HEPES, 2.5 mM CaCl₂, 1 mM MgCl₂, 0.5% BSA, pH=7.4

Binding wash buffer: 50 mM HEPES, 2.5 mM CaCl₂, 1 mM MgCl₂, 0.5M NaCl, 0.5% BSA, pH=7.4

Unifilter GFC filter plates: 50 μl of 0.5% polyethyleneimine was added to each well and left to equilibrate for four hours before use

Incubation plates: 96 well polypropylene plates, siliconised prior to use

Test Compounds: Compounds were dissolved in DMSO at a concentration of 1 mM. Final concentration of DMSO in the assay did not exceed 1%.

Peptide PYY (pancreatic polypeptide Y)—10 μM stock solution in binding buffer.

¹²⁵I PYY—10 μCi/ml stock solution, diluted 1:10 dilution, into binding buffer.

Assays were performed in 96 well microtitre plates. 10 μl of diluted test compound was added to each well of a plate, followed by 80 μl of membranes and 10 μl of radiolabelled ¹²⁵I PYY (0.01 μCi per well). Total and non-specific binding controls were included in each plate. The non-specific binding wells received 10 μl of Peptide PYY from the 10 μM stock solution, whilst the total binding wells received 10 μl of binding buffer. For each assay, a duplicate dose response of peptide PYY was included, top concentration 1 μM.

The plates were incubated for two hours at room temperature with mixing, and then filtered onto the pre-treated filter plates. The incubation plates were washed twice with 150 μl of cold binding wash buffer per well, then the filter plates were further washed with approximately 2.5 ml per well. The filter plates were dried overnight at room temperature, the bottoms were sealed, and 20 μl of Scintillant (Microscint 40, Canberra Packard) was added to each well. The tops of the plates were sealed and the plates were counted for 1 minute on a protocol set up for ¹²⁵I on a 96 well plate liquid scintillation counter (Top Count, Canberra Packard).

Compounds were considered to be active if they inhibited the binding by more than 50% at a concentration of 10 μM. Dose responses were carried out on all compounds found to be active (8 point curves in duplicate).

Although the pharmacological properties of the compounds of the formula (I) vary with structural change as expected, in general compounds of the formula (I) possess an IC₅₀ in the above test in the range, for example, 0.0002 to 200 μM.

In order to use a compound of the formula (I) or a pharmaceutically acceptable salt, prodrug or solvate thereof, for the therapeutic treatment (including prophylactic treatment) of mammals including humans, it is normally formulated in accordance with standard pharmaceutical practice as a pharmaceutical composition.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedures well known in the art.

Compositions for administration by insufflation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound of the formula (I) will naturally vary according to the nature and severity of the conditions, the age and sex of the animal or patient and the route of administration, according to well known principles of medicine.

In using a compound of the formula (I) for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred, particularly in tablet form. Typically, unit dosage forms will contain about 1 mg to 500 mg of a compound of this invention.

The compounds of this invention may be used in combination with other drugs and therapies used in the treatment of disease states which would benefit from antagonism at the neuropeptide Y5 receptor. For example, the compounds of the formula (I) could be used in combination with drugs and therapies used in the treatment of eating disorders.

If formulated as a fixed dose such combination products employ the compounds of this invention within the dosage range described herein and the other pharmaceutically-active agent within its approved dosage range. Sequential use is contemplated when a combination formulation is inappropriate.

Although the compounds of the formula (I) are primarily of value as therapeutic agents for use in a warm-blooded animal, such as a human being, they are also useful whenever it is required to antagonise binding at the neuropeptide Y5 receptor. Thus, they are useful as pharmacological standards for use in the development of new biological tests and in the search for new pharmacological agents.

According to a further aspect of the invention there is provided a pharmaceutical composition which comprises a compound of the formula (I) or a pharmaceutically acceptable salt, prodrug or solvate thereof, in association with a pharmaceutically acceptable diluent or carrier.

According to one feature of the invention there is provided the use of a compound of formula (I), or a pharmaceutically acceptable salt, prodrug or solvate thereof, as a medicament.

According to a further aspect of the present invention there is provided a compound of the formula (I), or a pharmaceutically acceptable salt, prodrug or solvate thereof, for use in a method of treatment of a warm-blooded animal by therapy.

According to another aspect of the invention there is provided a pharmaceutical composition comprising a compound of formula (I), or a pharmaceutically acceptable salt, prodrug or solvate thereof, in association with a pharmaceutically acceptable diluent or carrier for the treatment of disorders mediated by the neuropeptide Y5 receptor in a warm-blooded animal in need of such treatment.

Examples of “disorders mediated by the neuropeptide Y5 receptor” are eating disorders. Examples of eating disorders include obesity, bulimia or anorexia. Further examples of eating disorders include: obesity and related disorders, bulimia or anorexia. Examples of “related disorders” are diabetes, dyslipidaemia, hypertension and sleep disturbances. Preferably “related disorders” refers to diabetes.

According to a further aspect of the invention there is provided a pharmaceutical composition comprising a compound of formula (I), or a pharmaceutically acceptable salt, prodrug or solvate thereof, in association with a pharmaceutically acceptable diluent or carrier for the treatment of eating disorders in a warm-blooded animal.

According to a further aspect of the invention there is provided a pharmaceutical composition comprising a compound of formula (I), or a pharmaceutically acceptable salt, prodrug or solvate thereof, in association with a pharmaceutically acceptable diluent or carrier for use in promoting weight loss.

Preferably promoting weight loss would refer to promoting weight loss in a warm-bloodied animal. Preferably a warm-blooded animal is man.

According to a further aspect of the invention there is provided the use of a compound of formula (I), or a pharmaceutically acceptable salt, prodrug or solvate thereof, in the manufacture of a medicament for the treatment of eating disorders in a warm-blooded animal.

According to another feature of the invention there is provided the use of a compound of formula (I), or a pharmaceutically acceptable salt, prodrug or solvate thereof, in the manufacture of a medicament for promoting weight loss.

The use of a compound of formula (I), or a pharmaceutically acceptable salt, prodrug or solvate thereof, for the treatment of eating disorders in a warm-blooded animal.

The use of a compound of formula (I), or a pharmaceutically acceptable salt, prodrug or solvate thereof, for promoting weight loss.

According to a further aspect of the invention there is provided a method of treatment, in a warm-blooded animal, of eating disorders, comprising administering a therapeutically effective amount of a compound of formula (I), or a pharmaceutically acceptable salt, prodrug or solvate thereof.

According to a further aspect of the invention there is provided a method of promoting weight loss, comprising administering a therapeutically effective amount of a compound of formula (I), or a pharmaceutically acceptable salt, prodrug or solvate thereof.

### EXAMPLES

The invention will now be illustrated by the following non-limiting examples in which, unless stated otherwise:

(i) temperatures are given in degrees Celsius (° C.); operations were carried out at room or ambient temperature, that is, at a temperature in the range of 18-25° C.;

(ii) organic solutions were dried over anhydrous magnesium sulphate unless otherwise stated; evaporation of solvent was carried out using a rotary evaporator under reduced pressure (600-4000 Pascals; 4.5-30 mmHg) with a bath temperature of up to 60° C.;

(iii) chromatography means flash chromatography on silica gel (Merck Keiselgel ART 9385); thin layer chromatography (TLC) was carried out on silica gel plates; where a “Bond Elut” column is referred to, this means a column containing 20 g of silica, the silica being contained in a 70 ml disposable syringe and supported by a porous disc of 54 Å pore size, obtained from International Sorbent Technology under the name “ISOLUTE”; “ISOLUTE” is a registered trade mark; where a Biotage cartridge is referred to this means a cartridge containing KP-SIL™ silica, 60 Å, particle size 32-63 mM, supplied by Biotage, a division of Dyax Corp., 1500 Avon Street Extended, Charlottesville, Va. 22902, USA;

(iv) in general, the course of reactions was followed by TLC and reaction times are given for illustration only;

(v) final products had satisfactory proton nuclear magnetic resonance (NMR) spectra and/or mass spectral data;

(vi) yields are given for illustration only and are not necessarily those which can be obtained by diligent process development; preparations were repeated if more material was required;

(vii) when given, NMR data is in the form of delta values for major diagnostic protons, given in parts per million (ppm) relative to tetramethylsilane (TMS) as an internal standard, determined at 300 MHz using perdeuterio dimethyl sulphoxide (DMSO-d₆) as solvent unless otherwise indicated; s, singlet; d, doublet; dd, double doublet; t, triplet; tt, triple triplet; q, quartet; tq, triple quartet; sep, septuplet; m, multiplet; br, broad;

(viii) chemical symbols have their usual meanings; SI units and symbols are used;

(ix) solvent ratios are given in volume:volume (v/v) terms; and

(x) mass spectra were run with an electron energy of 70 electron volts in the chemical ionization (CI) mode using a direct exposure probe; where indicated ionization was effected by electron impact (EI), fast atom bombardment (FAB) or electrospray (ES); values for m/z are given; generally, only ions which indicate the parent mass are reported; unless otherwise stated the value for (M+H)⁺ is quoted;

(xi) unless stated otherwise compounds containing an asymmetrically substituted carbon and/or sulphur atom have not been resolved;

(xii) where a synthesis is described as being analogous to that described in a previous example the amounts used are the millimolar ratio equivalents to those used in the previous example;

(xvi) the following abbreviations have been used:

DMF N,N-dimethylformamide;

EDAC 1-[3-(dimethylamino)propyl]-3-ethylcarbodiimide hydrochloride;

DDQ 2,3-dichloro-5,6-dicyano-1,4-benzoquinone;

EtOH ethanol;

MeOH methanol;

EtOAc ethyl acetate; and

DCM dichloromethane.

### Example 1

### 2,4-Dimethyl-3-morpholinocarbonylamino-9-isopropyl-9H-carbazole

4-Morpholine carbonyl chloride (142 μl, 1.22 mmol) was added slowly to a solution of 3-amino-2,4-dimethyl-9-isopropyl-9H-carbazole (Method 14; 308 mg, 1.22 mmol) and triethylamine (170 μl, 1.22 mmol) in DCM (10 ml) and the mixture was stirred at room temperature under an argon atmosphere. Additional 4-morpholine carbonyl chloride (142 μl, 1.22 mmol) and triethylamine (170 μl, 1.22 mmol) were added after 16 h and 18 h respectively. The reaction mixture was heated at reflux for a further 64 h before purifying by chromatography on an Bond Elut cartridge (eluent—50% EtOAc/isohexane—75% EtOAc/isohexane) yielded the product as a white solid. Rf (50% EtOAc/isohexane) 0.10; NMR (CDCl₃) 8.18 (d, 1H), 8.52 (d, 1H), 7.40 (d, 1H), 7.20 (m, 2H), 5.90 (s, 1H), 4.96 (sep, 1H), 3.75 (m, 4H), 3.50 (m, 4H), 2.76 (s, 3H), 2.44 (s, 3H), 1.70 (d, 6H); m/z 366.76.

### Examples 2-12

Following the procedure of Example 1 and using the appropriate starting materials the following compounds were made.

| Ex | Compound | NMR | M/z | SM |
|---|---|---|---|---|
| 2 | 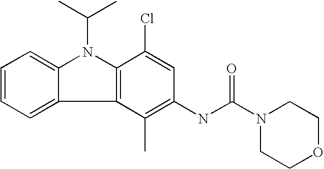 | (CDCl₃) 8.21 (d, 1H), 7.70 (d, 1H), 7.42 (t, 1H), 7.39 (s, 1H), 7.22 (t, 1H), 6.22 (sep, 1H), 6.13 (s, 1H), 3.75 (m, 4H), 3.50 (m, 4H), 2.72 (s, 3H), 1.70 (d, 6H) | 386.75 | Meth 24 |
| 3 | 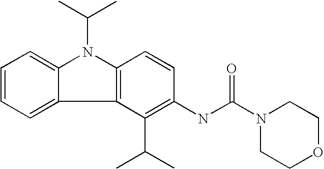 | (CDCl₃) 8.18 (d, 1H), 7.48 (d, 1H), 7.40-7.25 (m, 3H), 7.12 (t, 1H), 6.10 (brs, 1H), 4.94 (sep, 1H), 4.25 (brm, 1H), 3.69 (m, 4H), 3.44 (m, 4H), 1.65 (d, 6H), 1.49 (d, 6H) | 380.23 | Meth 20 |
| 4 | 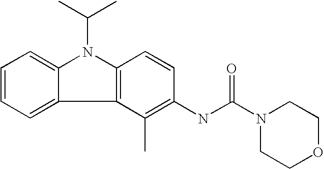 | (CDCl₃) 8.23 (d, 1H), 7.55 (d, 1H), 7.43 (t, 1H), 7.35 (m, 1H), 7.22 (m, 2H), 6.20 (s, 1H), 5.00 (sep. 1H), 3.73 (m, 4H), 3.49 (m, 4H), 2.77 (s, 3H), 1.71 (d, 6H) | 352.75 | Meth 21 |
| 5 | 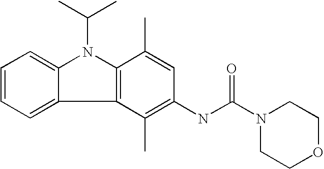 | 8.4 (m, 2H), 7.8 (d, 1H), 7.4 (t, 1H), 7.2 (t, 1H), 7.0 (s, 1H), 5.5 (m, 1H), 3.65 (m, 4H), 3.45 (m, 4H), 2.75 (s, 3H), 2.55 (s, 3H), 1.65 (d, 6H) | 367 | Meth 36 |
| 6 | 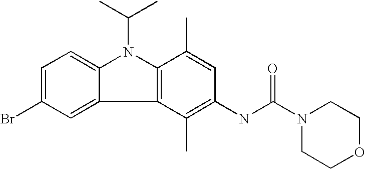 | 8.3 (d, 1H), 8.25 (s, 1H), 7.8 (d, 1H), 7.55 (dd, 1H), 7.1 (s, 1H), 5.55 (m, 1H), 3.7 (m, 4H), 3.5 (m, 4H), 2.8 (s, 3H), 2.55 (s, 3H), 1.7 (d, 6H) | 444/446 | Meth 37 |
| 7 | 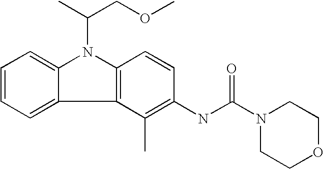 | 1.6 (d, 3H), 2.6 (s, 3H), 2.6 (s, 3H), 3.15 (s, 3H), 3.5 (m, 4H), 3.15 (m, 4H), 3.25 (m, 1H), 4.05 (t, 1H), 5.15 (m, 1H), 7.2 (m, 2H), 7.45, (m, 1H), 7.7 (d, 1H), 8.2 (d, 1H), 8.3 (s, 1H) | 382 | Meth 19 |
| 8 | 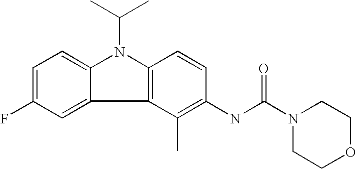 | 1.6 (d, 6H), 2.6 (s, 3H), 3.4 (m, 4H), 3.6 (m, 4H), 5.1 (m, 1H), 7.2-7.3 (m, 2H), 7.5 (d, 1H), 7.7 (d, 1H), 7.9 (d, 1H), 8.2 (s, 1H) | 369 | Meth 17 |
| 9 | 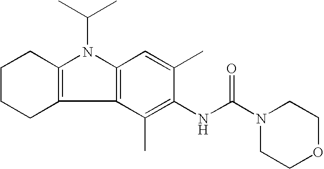 | (CDCl₃) 7.08 (s, 1H), 5.75 (s, 1H), 4.55 (sep, 1H), 3.72 (m, 4H), 3.46 (m, 4H), 2.97 (m, 2H), 2.71 (m, 2H), 2.53 (s, 3H), 2.32 (s, 3H), 1.82 (m, 4H), 1.53 (d, 6H) | 370.7 | Meth 15 |
| 10 | 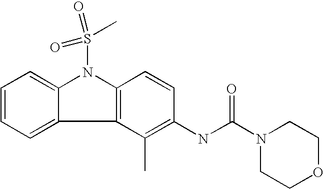 | 8.36 (s, 1H), 8.24 (d, 1H), 8.12 (d, 1H), 7.87 (d, 1H), 7.56 (dd, 1H), 7.56 (dd, 1H), 7.34 (d, 1H), 3.64 (m, 4H), 3.44 (m, 4H), 2.60 (s, 3H) | 388.38 | Meth 43 |
| 11 | 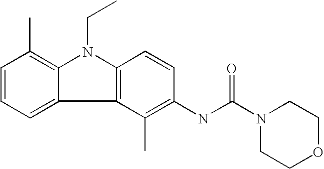 | 1.3 (t, 3H), 2.6 (s, 3H), 2.8 (s, 3H), 3.4 (m, 4H), 3.6 (m, 4H), 4.6 (q, 2H), 7.1 (t, 1H), 7.2 (m, 2H), 7.4 (d, 1H), 8.0 (d, 1H), 8.2 (s, 1H) | 352 | Meth 16 |
| 12 | 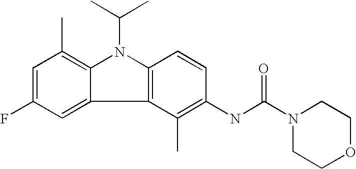 | (CDCl₃) 1.6 (d, 6H), 2.5 (s, 3H), 2.8 (s, 3H), 3.4 (m, 4H), 3.6 (m, 4H), 5.5 (m, 1H), 7.1 (d, 1H), 7.2 (d, 1H), 7.6 (d, 1H), 7.8 (d, 1H), 8.2 (s, 1H) | 284 | Meth 18 |

### Example 13

### 3-(3-Carbamoylpiperidin-1-ylcarbonylamino)-4-methyl-9-isopropyl-9H-carbazole

4-(Dimethylamino)pyridine (26 mg, 0.21 mmol) was added to a solution of 3-(2,2,2-trichloroacetamido)-4-methyl-9-isopropyl-9H-carbazole (Example 26; 0.86 g, 2.13 mmol) in EtOAc (30 ml). After stirring for 10 minutes, nipecotamide (300 mg, 2.34 mmol) was added portion-wise over 20 minutes. The reaction mixture was stirred for 2 hrs, pre-absorbed onto silica and loaded onto a suction column. The column was first eluted with EtOAc and then 10% MeOH/EtOAc. The required fractions were combined, dried and concentrated in vacuo to yield the title compound (0.77 g, 93%). NMR 8.15-8.20 (m, 2H), 7.7 (d, 1H), 7.3-7.5 (m, 3H), 7.15-7.20 (m, 2H), 6.8 (brs, 1H), 5.1 (sep, 1H), 4.0-4.20 (m, 2H), 2.75-2.95 (m, 2H), 2.6 (s, 3H), 2.2-2.4 (m, 1H), 1.9-1.95 (m, 1H), 1.35-1.70 (m, 9H).

### Example 14

### 3-(3-Carbamoylpiperidin-1-ylcarbonylamino)-2,4-dimethyl-9-isopropyl-9H-carbazole

3-(2,2,2-Trichloroacetamido)-2,4-dimethyl-9-isopropyl-9H-carbazole (Example 24; 0.200 g, 0.5 mmols), nicopecotamide (0.0966 g, 0.75 mmols) and sodium carbonate (0.32 g, 3.02 mmols) were placed in DMSO (5 ml) and stirred at 80° C. overnight. After cooling, the mixture was poured onto water, then extracted with EtOAc. The organic layers were combined, dried and evaporated to give a sticky yellow solid. This was purified by column chromatography (100% EtOAc—1%MeOH in EtOAc) to give a sticky white solid that was triturated with diethyl ether to give a cream solid (0.142 g, 69.5%). NMR 1.40-1.50 (m, 1H), 1.60 (m, 8H), 1.85-1.95 (m, 1H), 2.30 (m, 4H), 2.60 (s, 3H), 2.70-2.95 (m, 2H), 4.10-4.20 (m, 2H), 5.00-5.15 (m, 1H), 6.80 (brs, 1H), 7.10 (t, 1H), 7.30-7.40 (m, 3H), 7.65 (d, 1H), 7.90 (s, 1H), 8.10 (d, 1H); m/z 405 (M−H)⁻.

### Examples 15-21

Following the procedure of Example 14 and using 3-(2,2,2-trichloroacetamido)-2,4-dimethyl-9-isopropyl-9H-carbazole (Example 24) and the appropriate amine the following compounds were made.

| Ex | R¹ | NMR | M/z |
|---|---|---|---|
| 15 | 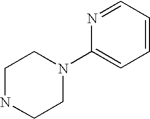 | 1.60 (d, 4H), 2.30 (s, 3H), 2.60 (s, 3H), 3.5 0 3.65 (m, 8H), 5.10 (m, 1H), 6.65 (t, 1H), 6.90 (d, 1H), 7.10 (t, 1H), 7.35-7.40 (m, 2H), 7.50 (m, 1H), 7.65 (d, 1H), 8.00 (s, 1H), 8.10 (m, 2H) | 440 (M − H)⁻ |
| 16¹ | 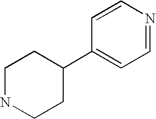 | 1.60 (d, 6H), 1.80 (m, 1H), 2.35 (s, 3H), 2.60 (s, 3H), 2.65-3.00 (4H), 4.20 (m, 1H), 4.30 (d, 2H), 5.10 (m, 1H), 7.15 (t, 1H), 7.25-7.40 (m, 4H), 7.65 (d, 1H), 7.95 (s, 1H), 8.10 (d, 1H), 8.5 (d, 2H) | 441 (M − H)⁻ |
| 17 | 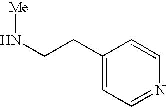 | 1.60 (d, 6H), 2.25 (s, 3H), 2.55 (s, 3H), 2.90 (t, 2H), 2.95 (s, 3H), 3.60 (t, 1H), 5.00-5.10 (m, 1H), 7.15 (t, 1H), 1.30-7.40 (m, 4H), 7.60-7.70 (m, 2H), 8.10 (d, 1H), 8.45 (d, 2H) | 413 (M − H)⁻ |
| 18² | 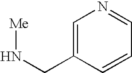 | 1.60 (d, 6H), 2.30 (s, 3H), 2.60 (s, 3H), 3.0 (s, 3H), 4.60 (s, 2H), 5.00-5.10 (m, 1H), 7.15 (t, 1H), 7.35-7.40 (m, 3H), 7.60-7.70 (m, 2H), 7.90 (s, 1H), 8.10 (d, 1H), 8.50 (d, 1H), 8.55 (d, 1H) | 399 (M − H)⁻ |
| 19 | 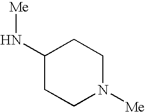 | 1.50 (m, 2H), 1.60 (d, 6H), 1.65-1.80 (m, 2H), 1.90-2.00 (m, 2H), 2.15 (s, 3H), 2.30 (s, 3H), 2.60 (s, 3H), 2.75-2.90 (m, 5H), 3.95-4.05 (m, 1H), 5.00-5.10 (m, 1H), 7.15 (m, 1H), 7.35-7.40 (m, 2H), 7.60-7.65 (m, 2H), 8.10 (d, 1H) | 407 (M − H)⁻ |
| 20 | 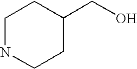 | 1.05-1.20 (m, 2H), 1.50-1.70 (m, 8H), 2.30 (s, 3H), 2.60 (s, 3H), 2.70-2.80 (m, 2H), 3.30 (m, 2H), 4.15 (d, 2H), 4.45 (t, 1H), 5.00-5.10 (m, 1H), 7.15 (t, 1H), 7.35-7.40 (m, 1H), 7.65 (d, 1H), 7.8p (s, 1H), 8.10 (d, 1H) | 392 (M − H)⁻ |
| 21 | 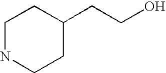 | 1.00-1.20 (m, 2H), 1.40 (q, 2H), 1.55-1.70 (m, 8H), 2.30 (s, 3H), 2.60 (s, 3H), 2.70-2.85 (m, 2H), 3.45 (q, 2H), 4.10 (d, 1H), 4.30 (t, 1H), 5.00-5.10 (m, 1H), 7.15 (t, 1H), 7.30-7.40 (m, 2H), 7.65 (d, 1H), 7.80 (s, 1H), 8.10 (d, 1H) | 406 (M − H)⁻ |

¹Amine: J. Am Chem. Soc., 1931, 53,, 277
²Amine: Can. J. Chem., 1978, 56, 3072

### Example 22

### 3-Pivaloylamino-4-methyl-9-ethyl-9H-carbazole

A solution of 3-Pivaloylamino-9-ethyl-9H-carbazole; 1.18 g, 4.0 mmol) in dry tetrahydrofuran (15 ml) was cooled to −30° C. under a stream of argon. 1.7M tert-butyllithium in pentane (5.15 ml, 8.8 mmol) was added cautiously to the stirred mixture over a period of 10 minutes, the mixture was allowed to warm to 0° C. over a period of 30 minutes and then re-cooled to −30° C. Iodomethane (596 mg, 4.2 mmol) was added slowly, the mixture stirred for 3 hours at ambient temperature then reduced in vacuo. The residue was purified by chromatography, eluting with 1:3 EtOAc in isohexane to give the title compound (214 mg). NMR 8.94 (s, 1H), 8.05 (d, 1H), 7.84 (s, 1H), 7.54 (d, 1H), 7.43 (s, 1H), 7.38 (t, 1H), 7.12 (t, 1H), 4.38 (q, 2H), 2.31 (s, 3H), 1.27 (m, 1H); m/z 309.

### Example 23

### 3-Pivaloylamino-2,4-dimethyl-9-isopropyl-9H-carbazole

Pivaloyl chloride (58 μl, 0.47 mmol) was added slowly to a solution of 3-amino-2,4-dimethyl-9-isopropyl-9H-carbazole (Method 14; 117 mg, 0.46 mmol) and triethylamine (66 μl, 0.47 mmol) in DCM (10 ml) stirred at 0° C. under an argon atmosphere. The reaction mixture was warmed to room temperature and stirred for 1 h before being extracted with DCM and washed with water. The organic layers were dried and the solvent was removed in vacuo to yield an off-white solid; 154 mg (100%). Rf (diethyl ether) 0.60; NMR 8.87 (s, 1H), 8.12 (d, 1H), 7.66 (d, 1H), 7.38 (m, 2H), 7.15 (t, 1H), 5.10 (sep, 1H), 2.58 (s, 3H), 2.30 (s, 3H), 1.63 (d, 6H), 1.30 (s, 9H); m/z 337.44.

### Examples 24-26

Following the procedure of Example 23 and using the appropriate starting materials the following compounds were made.

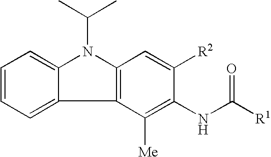

| Ex | R¹ | R² | NMR | M/z | SM |
|---|---|---|---|---|---|
| 24 | —CCl₃ | Me | (CDCl₃)1.70 (d, 6H), 2.45 (s, 3H), 2.75 (s. 3H), 5.0 | 397 | Meth |
| | | | (m, 1H), 7.20-7.30 (m, 2H), 7.45 (t, 1H), 7.55 (d, | (M − H)⁻ | 14 |
| | | | 1H), 8.00 (brs, 1H), 8.20 (d, 1H) | | |
| 25 | t-Bu | H | 9.04 (s, 1H), 8.17 (d, 1H), 7.69 (d, 1H), 7.49 (d, 1H), | 323.50 | Meth |
| | | | 7.40 (t, 1H), 7.17 (m, 2H), 5.10 (sep, 1H), 2.59 (s, 3H), | | 21 |
| | | | 1.62 (d, 6H), 1.27 (s, 9H) | | |
| 26 | —CCl₃ | H | (CDCl₃) 1.70 (d, 6H), 2.80 (s, 3H), 4.95-5.05 (m, | 397 | Meth |
| | | | 1H), 7.25 (t, 1H), 7.40-7.60 (m, 4H), 8.25 (d, 1H), | | 21 |
| | | | 8.30 (brs, 1H). | | |

### Example 27

### 3-(Tetrahydrofuran-3-ylcarbonylamino)-2,4-dimethyl-9-isopropyl-9H-carbazole

3-Amino-2,4-dimethyl-9-isopropyl-9H-carbazole (Method 14; 0.25 g, 0.99 mmols), dimethylaminopyridine (0.3394 g, 2.77 mmols) and tetrahydro-3-furoic acid (0.115 g, 0.99 mmols) were taken up in DCM (15 ml) and DMF (1 ml). To the stirred solution was added EDAC (0.2853 g, 1.48 mmols) and the reaction was stirred at room temperature overnight. The solvent was removed in vacuo, the residue taken up in EtOAc and washed thoroughly with water. The solution was dried and the solvent removed in vacuo. The resultant gold oil was purified by column chromatography (50%-80% EtOAc in hexane) to give a white solid (0.249 g, 72%). NMR 1.60 (d, 6H), 2.15 (q, 1H), 2.30 (s, 3H), 2.60 (s, 3H), 3.25 (m, 2H), 3.70-3.80 (m, 3H), 4.00 (t, 1H), 5.20 (m, 1H), 7.15 (t, 1H), 7.25-7.4 (m, 2H), 7.70 (d, 1H), 8.10 (d, 1H), 9.35 (s, 1H); m/z 349 (M−H)⁻.

### Examples 28-36

Following the procedure of Example 27 and using the appropriate starting materials (Commercially available unless otherwise indicated) the following compounds were made.

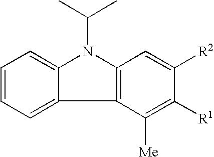

| Ex | R¹ | R² | NMR | M/z | SM |
|---|---|---|---|---|---|
| 28 | 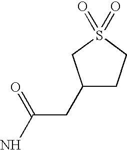 | Me | (CDCl₃) 1.65-1.8 (m, 7H), 1.95-2.15 (m, 1H), 2.40 (s, 3H), 2.45 (s, 1H), 2.65 (d, 1H), 2.7 (s, 3H), 2.8 (s, 1H), 2.9-3.25 (m, 3H), 3.35-3.4 (m, 1H), 4.95-5.05 (m, 1H), 6.80 (d, 1H), 7.20-7.30 (m, 2H), 7.40-7.6 (m, 2H), 8.10-8.20 (t, 1H) | 442 (M − H)⁻ | Meth 14 |
| 29 | 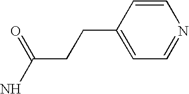 | Me | 1.60 (d, 6H), 2.20 (s, 3H), 2.75 (t, 2H), 3.00 (t, 2H), 3.25 (s, 3H), 5.05 (m, 1H), 7.15 (t, 1H), 7.30-7.40(4H), 7.65 (d, 1H), 8.10 (d, 1H), 8.50 (d, 2H), 9.25 (s, 1H) | 384 (M − H)⁻ | Meth 14 |
| 30 | 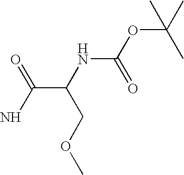 | H | 9.55 (s, 1H), 8.20 (d, 1H), 7.70 (d, 1H), 7.50 (d, 1H), 7.42 (t, 1H), 7.22 (d, 2H), 7.19 (t, 1H), 6.90 (d, 1H), 5.15 (sep, 1H), 4.30-4.43 (m, 1H), 3.60-3.65 (m, 2H), 3.52 (s, 3H), 2.61 (s, 3H), 1.62 (d, 6H), 1.40 (s, 9H) | 440.56 | Meth 21 |
| 31 | 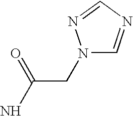 | H | 9.90 (s, 1H), 8.55 (s, 1H), 8.20 (d, 1H), 8.00 (s, 1H), 7.72 (d, 1H), 7.52 (d, 1H), 7.40 (t, 1H), 7.30 (d, 1H), 7.10 (t, 1H), 5.20 (s, 2H), 5.10 (sep, 1H), 2.62 (s, 3H), 1.60 (d, 6H) | 348.51 | Meth 21 |
| 32 | 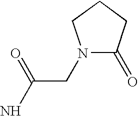 | H | 9.60 (s, 1H), 8.20 (d, 1H), 7.70 (d, 1H), 7.50 (d, 1H), 7.40 (t, 1H), 7.25 (d, 1H), 7.10 (t, 1H), 5.05 (sep, 1H), 4.05 (s, 2H), 3.50 (t, 2H), 2.62 (s, 3H), 2.15 (t, 2H), 1.95-2.05 (m, 2H), 1.62 (d, 6H) | 364.52 | Meth 21 |
| 33 | 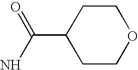 | H | 9.40 (s, 1H), 8.18 (d, 1H), 7.70 (d, 1H), 7.50 (d, 1H), 7.40 (t, 1H), 7.22 (d, 1H), 7.19 (t, 1H), 5.05 (sep, 1H), 3.95-4.00 (m, 2H), 3.20 (dt, 2H), 2.60 (s, 3H), 1.70-1.80 (m, 4H), 1.62 (d, 6H) | 351.52 | Meth 21 |
| 34 | 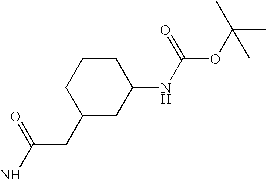 | H | 9.41 (s, 1H), 8.18 (d, 1H), 7.70 (d, 1H), 7.50 (d, 1H), 7.40 (t, 1H), 7.10-7.23 (m, 2H), 6.70 (d, 1H), 5.10 (sep, 1H), 3.65 (m, 1H), 2.60 (s, 3H), 2.20-2.40 (m, 3H), 1.65-1.95 (m, 2H), 1.63 (d, 6H), 1.50-1.60 (m, 2H), 1.40 (s, 9H), 0.90-1.25 (m, 3H) | 478.61 | Meth 21 and Meth 47 |
| 35 | 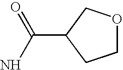 | H | 9.55 (s, 1H), 8.20 (d, 1H), 7.70 (d, 1H), 7.50 (d, 1H), 7.40 (t, 1H), 7.22 (d, 1H), 7.19 (t, 1H), 5.10 (sep, 1H), 4.00 (t, 1H), 3.70-3.92 (m, 3H), 3.15-3.30 (m, 1H), 3.60 (s, 3H), 2.10 (q, 2H), 1.62 (d, 6H) | 337.51 | Meth 21 |
| 36 | 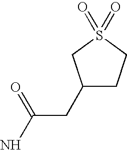 | H | 9.55 (s, 1H), 8.20 (d, 1H), 7.70 (d, 1H), 7.50 (d, 1H), 7.40 (t, 1H), 7.15-7.30 (m, 2H), 5.10 (sep. 1H), 3.00-3.35 (m, 3H), 2.75-2.95 (m, 2H), 2.65 (s, 3H), 2.60-2.65 (m, 2H), 2.30-2.40 (m, 1H), 1.90-2.00 (m, 1H), 1.62 (d, 6H) | 399.47 | Meth 21 |

### Example 37

### 3-(3-Carbamoylpiperidin-1-yloxalylamino)-4-methyl-9-isopropyl-9H-carbazole

3-Trichloroacetamidodimethyl-9-isopropyl-9H-carbazole (Example 26; 400 mg, 1.03 mmols), nipecotamide (119.2 mg, 1.56 mmols) and sodium carbonate (659.9 mg, 6.22 mmols) were added to DMSO (10 ml) and the reaction mixture heated to 80° C. overnight. After cooling, the reaction mixture was poured onto water, then extracted with EtOAc. The organic layers were combined, dried and evaporated to give a dark brown oil. This was purified by column chromatography (50% EtOAc in hexane) to give a dark yellow solid 272.2 mg (43.5%). NMR 1.35-1.60 (m, 2H), 1.60 (d, 6H), 1.75 (m, 1H), 2.20-2.40 (m, 1H), 2.65 (m, 3H), 2.70-2.85 (m, 2H), 3.10-3.20 (m, 1H), 3.80-3.90 (m, 1H), 4.20-4.40 (m, 1H), 5.05-5.15 (m, 1H), 6.90 (brs, 1H), 7.20 (t, 1H), 7.25-7.30 (m, 1H), 7.35-7.45 (m, 3H), 7.55 (d, 1H), 7.70 (m, 1H), 8.20 (d, 1H); m/z ESP 421.

### Examples 38-39

Following the procedure of Example 37 and using Example 26 and the appropriate amine the following compounds were made.

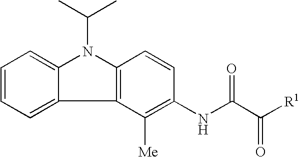

| Ex | R¹ | NMR | M/z |
|---|---|---|---|
| 38¹ | 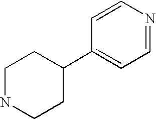 | 1.60 (d, 6H), 1.70-1.80 (m, 1H), 1.85-1.95 (m, 1H), 2.65 (s, 3H), 2.80-3.00 (m, 2H), 3.25-3.35 (m, 1H), 4.00-4.10 (m, 1H), 4.50 (m, 1H), 5.05-5.20 (m, 2H), 7.20 (t, 1H), 7.20 (m, 3H), 7.40 (t, 1H), 7.55 (d, 1H), 7.70 (d, 1H), 8.20 (d, 1H), 8.50 (m, 2H) | 455 |
| 39 | 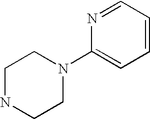 | (CDCl₃) 1.70 (d, 6H), 2.80 (s, 3H), 3.60-3.75 (m, 4H), 3.85-3.95 (m, 2H), 4.40-4.50 (m, 2H), 4.95-5.05 (m, 1H), 6.60-6.70 (m, 2H), 7.20-7.25 (m, 1H), 7.40-7.60 (m, 4H), 7.65 (d, 1H), 8.20-8.30 (m, 2H), 9.20 (brs, 1H) | 456 |

¹Amine: J. Am. Chenz. Soc., 1931, 53, , 277

### Example 40

### 3-(3-Methoxy-2-aminopropionamido)-4-methyl-9-isopropyl-9H-carbazole

3-[3-Methoxy-2-(t-butoxycarbonylamino)propionamido]4-methyl-9-isopropyl-9H-carbazole (Example 30; 270.9 mg, 0.62 mmol) was dissolved in DCM (10 ml) and cooled to 0° C. Trifluoroacetic acid (0.5 ml, 6.5 mmol) was added dropwise and the mixture was allowed to warm to room temperature and stirred 12 h under argon. Water (20 ml) was added to the reaction mixture and then basified with aqueous potassium carbonate solution. The aqueous mixture was extracted with DCM (3×20 ml). The combined organic extracts were dried and concentrated in vacuo to yield the title compound (0.2016 g, 96%). NMR 8.20 (d, 1H), 7.70 (d, 1H), 7.50 (d, 1H), 7.35-7.45 (m, 2H), 7.18 (t, 1H), 5.0-5.20 (m, 1H), 3.50-3.62 (m, 3H), 3.35 (s, 3H), 2.65 (s, 3H), 1.6 (d, 6H); m/z 340.59.

### Example 41

### 3-[2-(3-Aminocyclohexyl)acetamido]-4-methyl-9-isopropyl-9H-carbazole

The title compound was prepared from 3-{2-[3-(t-butoxycarbonylamino)cyclohexyl] acetamido}-4-methyl-9-isopropyl-9H-carbazole (Example 34) by the procedure of Example 40. NMR 9.40 (s, 1H), 8.19 (d, 1H), 7.70 (d, 1H), 7.50 (d, 1H), 7.40 (t, 1H), 7.10-7.25 (m, 2H), 5.10 (sep, 1H), 3.30 (brs, 2H), 3.00-3.05 (m, 1H), 2.65 (s, 3H), 2.20-2.30 (m, 2H), 1.70-1.95 (m, 2H), 1.65 (d, 6H), 1.10-1.50 (m, 6H), 0.9-1.0 (m, 1H); m/z 378.65.

### Example 42

### 3-(4-Hydroxypiperidin-1-ylcarbonylamino)-4-methyl-6-fluoro-9-isopropyl-9H-carbazole

A solution of 3-amino-4-methyl-6-fluoro-9-isopropyl-9H-carbazole (Method 17; 256 mg, 1 mmol) in DCM (4.5 ml) containing diisopropylethylamine (197 μl, 1.1 mmol) was added to a stirred solution of triphosgene (115 mg, 0.35 mmol) in DCM (5 ml) under argon over a period of 30 mins, the solution was stirred for a further 5 mins then a 2.5 ml aliquot was removed and added to a stirred solution of 4-hydroxypiperidine (30 mg, 0.3 mmol) in DCM (2 ml) containing diisopropylethylamine (47 μl, 0.27 μmol) under argon and stirred overnight. The solution was diluted with EtOAc (20 ml), washed with water (10 ml), 3M citric acid (10 ml), water (10 ml) and saturated brine (10 ml), then dried and evaporated to dryness. After trituration with diethyl ether and filtration the title compound was obtained as white solid (92 mg). NMR (CDCl₃) 1.5 (m, 2H), 1.6 (d, 6H), 1.9 (m, 2H), 2.7 (s, 3H), 3.2 (m, 2H), 3.9 (m, 3H), 4.9 (m, 1H), 6.3 (s, 1H), 7.2 (t, 1H), 7.3 (s, 1H), 7.4 (d, 1H), 7.9 (d, 1H),: m/z 384 (M⁺).

### Examples 43-45

Following the procedure of Example 42 and using 3-amino-4-methyl-6-fluoro-9-isopropyl-9H-carbazole (Method 17) and the appropriate amine the following compounds were made.

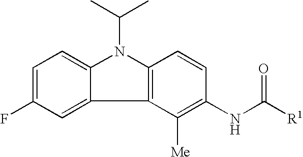

| Ex | R¹ | NMR | M/z |
|---|---|---|---|
| 43 | 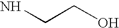 | (CDCl₃) 1.6 (d, 6H), 2.8 (s, 3H), 3.4 (m, 2H), 3.7 (t, 2H), 4.8 (s, 1H), 5.0 (m, 1H), 6.2 (s, 1H), 7.2 (t, 1H), 7.4 (m, 1H), 7.5 (d, 1H), 7.9 (d, 1H) | 344 (M⁺) |
| 44 | 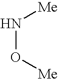 | (CDCl₃) 1.6 (d, 6H), 2.7 (s, 3H), 3.2 (s, 3H), 3.8 (s, 3H), 4.9 (m, 1H), 7.2 (t, 1H), 7.3 (d, 1H), 7.4 (d, 1H), 7.6 (m, 2H), 7.9 (d, 1H) | 344 (M⁺) |
| 45 | 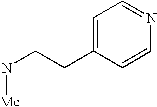 | (CDCl₃) 1.6 (d, 6H), 2.7 (s, 3H), 2.9 (t, 2H), 3.0 (s, 3H), 4.9 (m, 1H), 6.0 (s, 1H), 7.1-7.2 (m, 3H), 7.3 (m, 1H), 7.4 (d, 1H), 7.9 (d, 1H), 8.5 (d, 2H) | 419 (M⁺) |

### Examples 46-99

3-(Phenoxycarbonylamino)-2,4-dimethyl-9-isopropyl-9H-carbazole (Method 48; 100 mg, 0.28 mmol) was stirred with the appropriate amine (0.28 mmol) and triethylamine (0.28 mmol) in dry tetrahydrofuran (2 ml) at 55° C. for 64 hours. After cooling to room temperature, water (6 ml) was added and the resulting precipitate filtered off and washed with 2M sodium hydroxide solution (2×2 ml) then water (2 ml).

The following compounds were prepared by the above procedure using 3-(phenoxycarbonylamino)-2,4-dimethyl-9-isopropyl-9H-carbazole (Method 48) or 3-(phenoxycarbonylamino)-4-methyl-9-isopropyl-9H-carbazole (Method 49).

| Ex | Compound | MS |
|---|---|---|
| 46 | 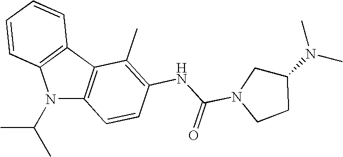 | 239 |
| 47 | 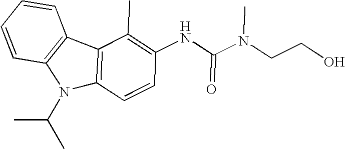 | 338 |
| 48 | 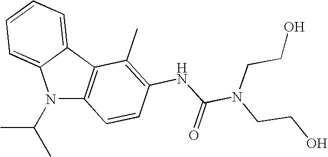 | 370 |
| 49 | 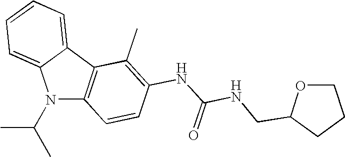 | 366 |
| 50 | 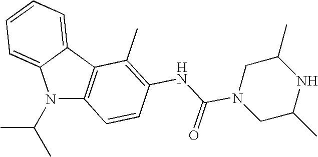 | 379 |
| 51 | 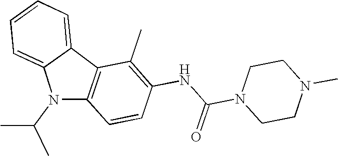 | 365 |
| 52 | 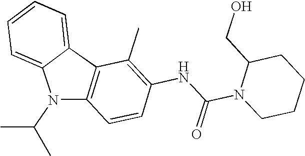 | 380 |
| 53 | 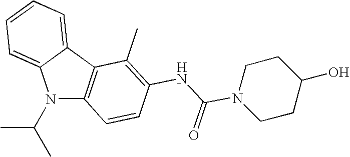 | 366 |
| 54 | 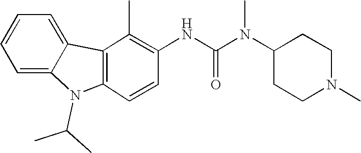 | 393 |
| 55 | 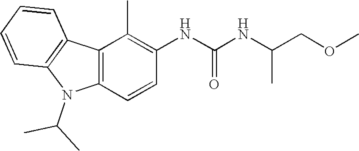 | 354 |
| 56 | 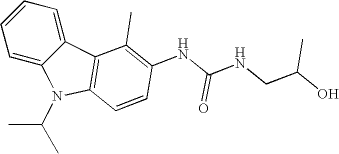 | 340 |
| 57 | 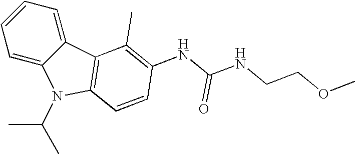 | 340 |
| 58 | 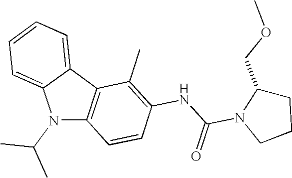 | 380 |
| 59 | 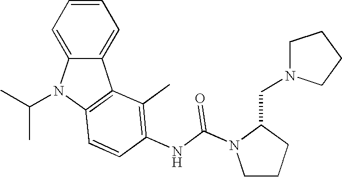 | 419 |
| 60 | 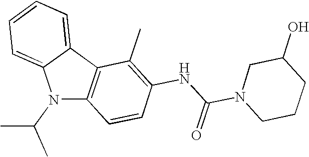 | 366 |
| 61 | 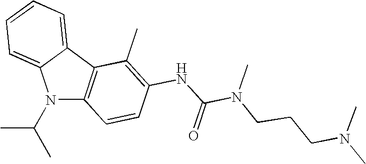 | 381 |
| 62 | 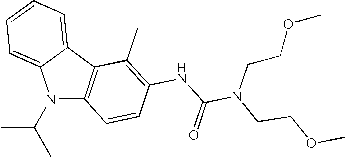 | 398 |
| 63 | 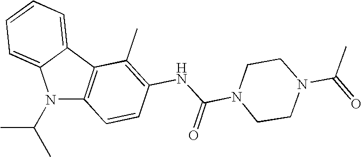 | 393 |
| 64 | 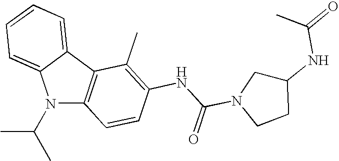 | 393 |
| 65 | 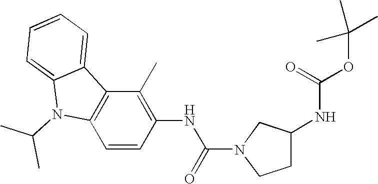 | 451 |
| 66 | 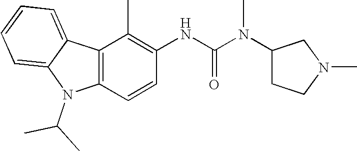 | 379 |
| 67 | 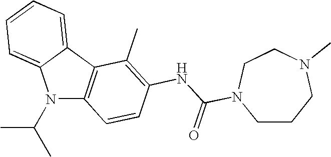 | 379 |
| 68 | 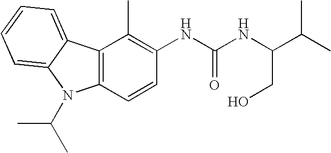 | 368 |
| 69 | 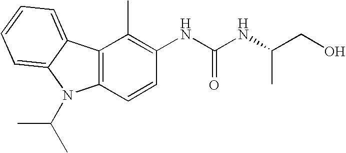 | 340 |
| 70 | 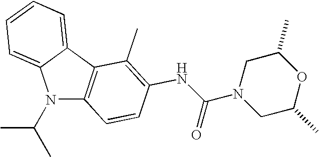 | 380 |
| 71 | 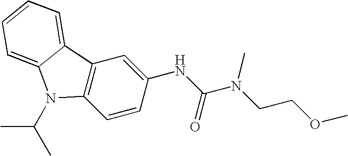 | 354 |
| 72 | 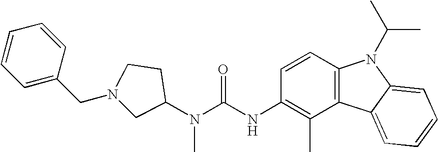 | 455 |
| 73 | 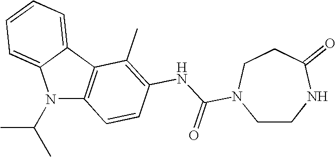 | 379 |
| 74 | 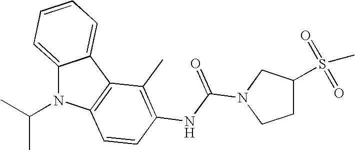 | 414 |
| 75 | 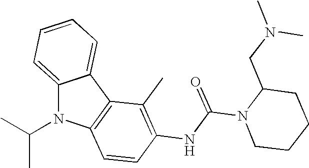 | 407 |
| 76 | 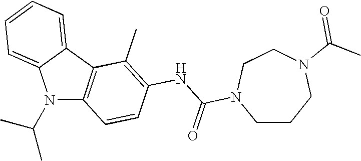 | 407 |
| 77 | 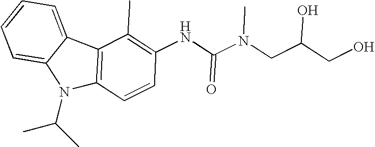 | 370 |
| 78 | 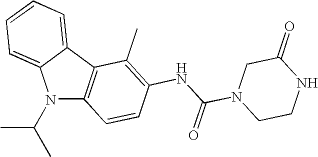 | 365 |
| 79 | 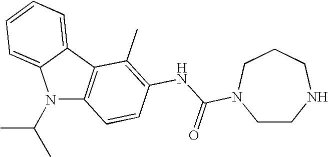 | 365 |
| 80 | 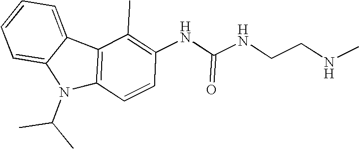 | 339 |
| 81 | 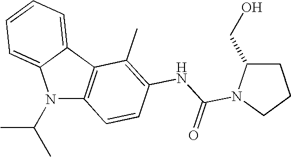 | 366 |
| 82 | 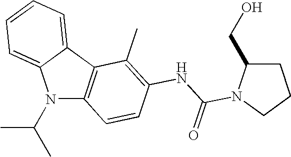 | 366 |
| 83 | 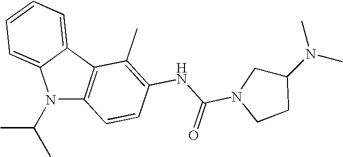 | 379 |
| 84 | 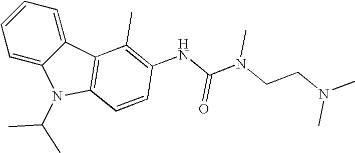 | 367 |

| Ex | Compound | NMR | MS |
|---|---|---|---|
| 85 | 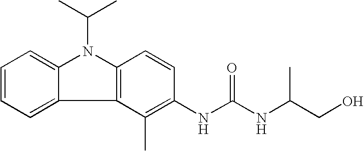 | 1.09 (d, 3H), 1.63 (d, 6H), 2.65 (s, 3H), 3.39 (m, 2H), 3.69 (dt, 1H), 4.77 (br s, 1H), 5.10 (sept, 1H), 6.00 (d, 1H), 7.16 (t, 1H), 7.43 (m, 3H), 7.69 (d, 1H), 7.82 (s, 1H), 8.19 (d, 1H) | 340.32 |
| 86 | 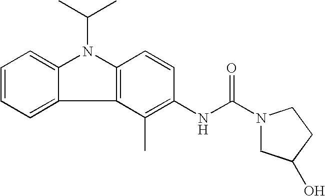 | 1.63 (d, 6H), 1.83 (m, 1H), 1.96 (m, 1H), 2.65 (s, 3H), 3.31 (m, 2H), 3.48 (m, 3H), 4.30 (m, 1H), 4.99 (br s, 1H), 5.10 (sept, 1H), 7.20 (m, 2H), 7.42 (t, 1H), 7.48 (d, 1H), 7.72 (d, 1H), 7.83 (s, 1H), 8.18 (d, 1H) | 352.32 |
| 87 | 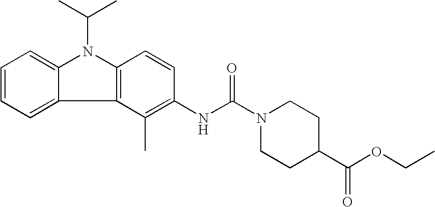 | 1.20 (t, 3H), 1.54 (m, 2H), 1.61 (d, 6H), 1.83 (m, 2H), 2.59 (s, 3H), 2.93 (m, 2H), 3.30 (m, 1H), 4.05 (m, 4H), 5.10 (sept, 1H), 7.17 (m, 2H), 7.39 (t, 1H), 7.46 (d, 1H), 7.67 (d, 1H), 8.17 (m, 2H) | 422.33 |
| 88 | 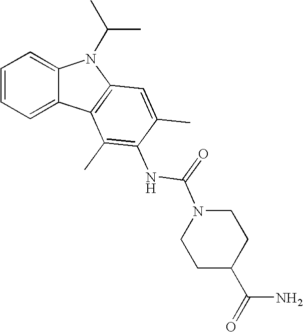 | 1.50 (m, 2H), 1.62 (d, 6H), 1.73 (m, 2H), 2.35 (s, 3H), 2.62 (s, 3H), 2.84 (m, 2H), 3.40 (m, 1H), 4.15 (m, 2H), 5.11 (sept, 1H), 6.82 (s, 1H), 7.17 (m, 1H), 7.30 (s, 1H), 7.40 (m, 2H), 7.68 (d, 1H), 7.90 (s, 1H), 8.13 (d, 1H) | 407.28 |
| 89 | 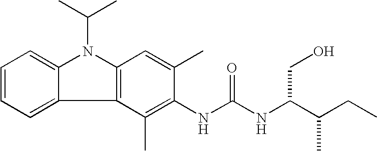 | 0.87 (m, 6H), 1.20 (m, 3H), 1.63 (d, 6H), 2.36 (s, 3H), 2.65 (s, 3H), 3.45 (m, 3H), 4.67 (br s, 1H), 5.11 (sept, 1H), 6.04 (br s, 1H), 7.17 (t, 1H), 7.38 (m, 2H), 7.50 (s, 1H), 7.68 (d, 1H), 8.13 (d, 1H) | 396.65 |
| 90 | 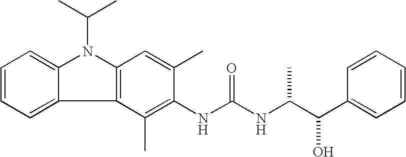 | 0.87 (m, 4H), 1.63 (d, 6H), 2.33 (s, 3H), 2.62 (s, 3H), 3.42 (m, 1H), 3.88 (m, 1H), 4.67 (br s, 1H), 5.08 (sept, 1H), 6.27 (br s, 1H), 7.28 (m, 8H), 7.62 (s, 1H), 7.68 (d, 1H), 8.13 (d, 1H) | 430.63 |
| 91 | 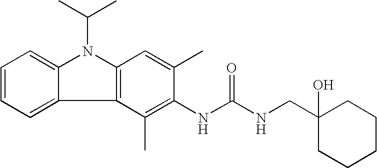 | 1.42 (m, 10H), 1.63 (d, 6H), 2.32 (s, 3H), 2.62 (s, 3H), 3.88 (m, 1H), 4.42 (br s, 1H), 5.08 (sept, 1H), 7.13 (t, 1H), 7.36 (m, 2H), 7.66 (m, 2H), 8.13 (d, 1H) | 408.32 |
| 92 | 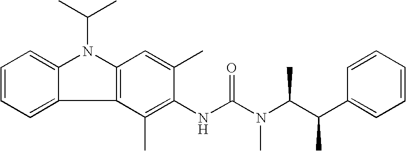 | 1.22 (d, 3H), 1.62 (d, 6H), 2.33 (s, 3H), 2.63 (s, 3H), 2.83 (s, 3H), 4.45 (m, 1H), 4.60 (d, 1H), 5.08 (sept, 1H), 7.13 (t, 1H), 7.32 (m, 6H), 7.48 (s, 2H), 7.67 (d, 1H), 8.10 (d, 1H) | 444.31 |
| 93 | 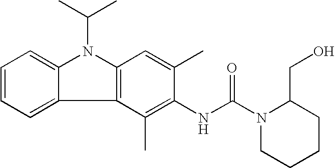 | 1.49 (m, 6H), 1.62 (d, 6H), 1.82 (m, 1H), 2.32 (s, 3H), 2.62 (s, 3H), 2.88 (m, 1H), 3.61 (m, 2H), 4.00 (m, 1H), 4.25 (m, 1H), 5.10 (sept, 1H), 7.15 (t, 1H), 7.40 (m, 2H), 7.66 (d, 1H), 7.75 (s, 1H), 8.13 (d, 1H) | 394.32 |
| 94 | 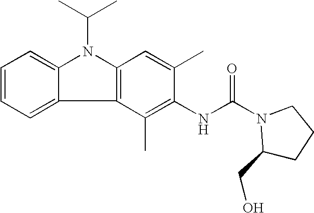 | 1.62 (d, 6H), 1.90 (m, 4H), 2.35 (s, 3H), 2.63 (s, 3H), 3.50 (m, 5H), 3.97 (m, 1H), 5.10 (sept, 1H), 7.13 (t, 1H), 7.38 (m, 2H), 7.66 (d, 1H), 7.81 (s, 1H), 8.13 (d, 1H) | 380.34 |
| 95 | 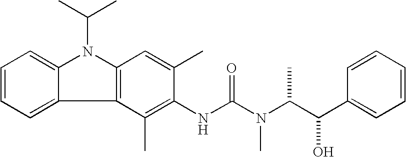 | 1.22 (d, 3H), 1.62 (d, 6H), 2.33 (s, 3H), 2.63 (s, 3H), 2.83 (s, 3H), 4.45 (m, 1H), 4.60 (d, 1H), 5.08 (sept, 1H), 5.49 (d, 1H), 7.13 (t, 1H), 7.32 (m, 6H), 7.48 (s, 2H), 7.67 (d, 1H), 8.10 (d, 1H) | 444.32 |
| 96 | 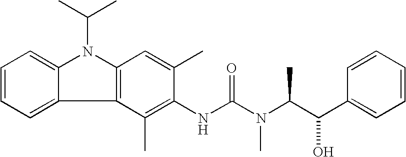 | 1.01 (d, 3H), 1.62 (d, 6H), 2.29 (s, 3H), 2.60 (s, 3H), 2.89 (s, 3H), 4.40 (m, 1H), 4.60 (m, 1H), 5.11 (sept, 1H), 7.13 (t, 1H), 7.32 (m, 6H), 7.67 (m, 2H), 8.13 (d, 1H) | 444.32 |
| 97 | 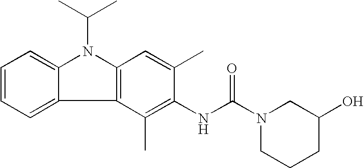 | 1.39 (m, 4H), 1.62 (d, 6H), 1.93 (m, 1H), 2.32 (s, 3H), 2.60 (s, 3H), 2.67 (m, 1H), 2.87 (m, 1H), 3.89 (m, 1H), 4.04 (m, 1H), 4.89 (br s, 1H), 5.11 (sept, 1H), 7.15 (t, 1H), 7.38 (m, 2H), 7.68 (d, 1H), 7.78 (s, 1H), 8.13 (d, 1H) | 380.34 |
| 98 | 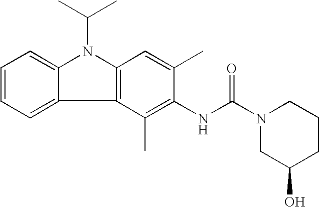 | 1.39 (m, 4H), 1.62 (d, 6H), 1.93 (m, 1H), 2.32 (s, 3H), 2.67 (m, 1H), 2.87 (m, 1H), 3.89 (m, 1H), 4.04 (m, 1H), 4.89 (br s, 1H), 5.11 (sept, 1H), 7.15 (t, 1H), 7.38 (m, 2H), 7.68 (d, 1H), 7.78 (s, 1H), 8.13 (d, 1H) | 380.35 |
| 99 | 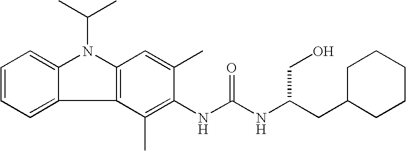 | 1.22 (m, 13H), 1.59 (d, 6H), 1.91 (m, 1H), 2.40 (s, 3H), 2.70 (s, 3H), 3.39 (m, 2H), 3.79 (m, 1H), 4.71 (br s, 1H), 5.11 (sept, 1H), 7.21 (t, 1H), 7.44 (m, 3H), 7.73 (d, 1H), 8.20 (d, 1H) | 436.69 |

### EXAMPLES 100-103

4-(Dimethylamino)pyridine (9 mg, 0.074 mmol) was added to a solution of 3-(4-nitrophenoxycarbonylamino)-2,4-dimethyl-9-isopropyl-9H-carbazole (Method 50; 300 mg, 0.72 mmol) in ethyl acetate (15 ml) and stirred for 10 minutes at room temperature. The relevant amines (0.792 mmol) were added and the reaction mixture was stirred for 3 hours. Ethyl acetate (10 ml) was added and the mixture was washed with potassium carbonate solution (3×20 ml). The ethyl acetate was concentrated in vacuo and the crude material was dissolved in dichloromethane (10 ml) to which was added TBD-methyl polystyrene (0.72 mmol). This was stirred for 12 hours, filtered, dried and concentrated in vacuo to yield the required products;

| Ex | Compound | NMR | MS |
|---|---|---|---|
| 100 | 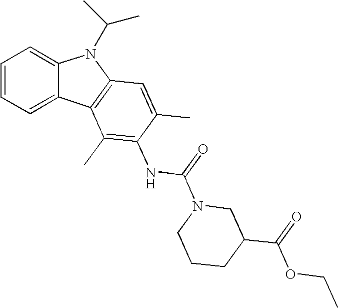 | 1.4-1.75 (m, 9H), 2.32 (s, 3H), 2.6 (s, 3H) 2.92-3.1 (m, 2H), 3.9-4.2 (m, 4H), 5.10 (sept, 1H), 7.15 (t, 1H), 7.3-7.4 (m, 2H), 7.65 (d, 1H), 7.95 (s, 1H), 8.10 (d, 1H) | 436.38 |
| 101 | 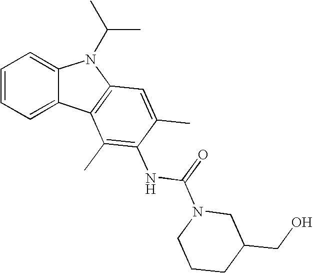 | 1.55-1.7 (m, 8H), 1.72-1.85 (m, 1H), 2.32 (s, 3H), 2.6 (s, 3H), 2.82-2.95 (m, 1H), 3.22-3.35 (m, 4H), 3.92-4.12 (m, 2H), 4.45 (t, 1H), 5.1 (sept, 1H), 7.15 (t, 1H), 7.3-7.42 (m, 2H), 7.65 (d, 1H), 7.8 (s, 1H), 8.1 (d, 1H) | 394.36 |
| 102 | 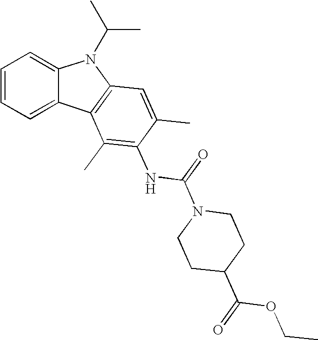 | 1.1 (t, 3H), 1.45-1.65 (m, 9H), 1.78-1.92 (m, 2H), 2.32 (s, 3H), 2.6 (s, 3H), 2.88-3.2 (m, 2H), 4.0-4.12 (m, 4H), 5.08 (sept, 1H), 7.12 (t, 1H), 7.3-7.4 (m, 2H), 7.65 (d, 1H), 7.9 (s, 1H), 8.12 (d, 1H) | 436.37 |
| 103 | 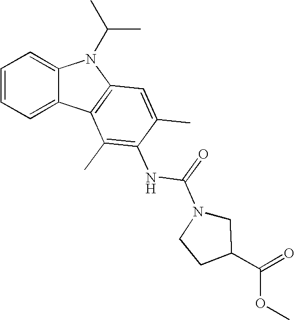 | (CDCl₃) 1.72 (d, 6H), 2.2-2.35 (m, 2H), 2.5 (s, 3H), 2.82 (s, 3H), 3.1-3.25 (m, 1H), 3.45-3.6 (m, 1H), 3.65-3.9 (m, 6H), 5.0 (sept, 1H), 5.75 (s, 1H), 7.15-7.3 (m, 2H), 7.45 (t, 1H), 7.55 (d, 1H), 8.25 (d, 1H) | 408.26 |

### Example 104

### 3-(4Carboxypiperidin-1-ylcarbonylamino)-4-methyl-9-isopropyl-9H-carbazole

2M Solution of NaOH (30 ml) was added to a stirred solution of 3-(4-ethoxycarbonyl-piperidin-1-ylcarbonylamino)4methyl-9-isopropyl-9H-carbazole (Example 87; 1.6 g, 3.8 mmol) in ethanol (50 ml). The mixture was stirred at room temperature for a total of 2 h 40 min before acidification with 2M HCl solution followed by extraction with EtOAc (2×200 ml) and washing with water (2×200 ml). Drying and solvent removal yielded the product as a brown gum (1.47 g, 98%). NMR 1.54 (m, 2H), 1.62 (d, 6H), 1.84 (m, 2H), 2.60 (s, 3H), 2.93 (m, 2H), 3.29 (m, 1H), 4.03 (m, 4H), 5.09 (sept, 1H), 7.17 (m, 2H), 7.41 (m, 2H), 7.67 (d, 1H), 8.17 (m, 2H); m/z 394.30.

### Example 105

### 3-[3-(N,N-Dimethylcarbamoyl)piperidin-1-ylcarbonylamino]-4-methyl-9-isopropyl-9H-carbazole

3-(3-Carboxypiperidin-1-ylcarbonylamino)-4-methyl-9-isopropyl-9H-carbazole (Example 104; 200 mg, 0.51 mmol) was stirred in dry THF (4 ml) with EDAC (108 mg, 0.56 mmol) and 4-dimethylaminopyridine (10 mg). Dimethylamine (2M soln. in THF, 280 μl) was added and the mixture stirred at room temperature for 16 h. The reaction mixture was extracted with EtOAc (2×200 ml) and washed with water (2×200 ml). Solvent was removed and chromatography (eluent—MeOH/EtOAc 1:9) yielded the product as a white solid (65 mg, 30%). NMR 1.50 (m, 2H), 1.62 (d, 6H), 1.67 (m, 1H), 2.60 (s, 3H), 2.83 (s, 3H), 2.90 (m, 4H), 3.05 (s, 3H), 4.15 (m, 2H), 5.09 (sept, 1H), 7.17 (m, 2H), 7.41 (m, 2H), 7.67 (d, 1H), 8.17 (m, 2H); m/z 421.33.

### Example 106

### 3-[3-(N-methylcarbamoyl)piperidin-1-ylcarbonylamino]-4-methyl-9-isopropyl-9H-carbazole

3-(3-Carboxypiperidin-1-ylcarbonylamino)-4-methyl-9-isopropyl-9H-carbazole (Example 104; 177 mg, 0.45 mmol) was stirred in dry THF with triethylamine (70 μl, 0.5 mmol) at room temperature. Ethylchloroformate (48 μl, 0.5 mmol) was added slowly before stirring for 1 h. Methylamine (2M soln. in THF, 1 ml) was added and the mixture stirred at room temperature for 3 h. The reaction mixture was extracted with EtOAc (2×200 ml) and washed with saturated potassium carbonate solution. The organic layer was then backwashed with 2M HCl solution before drying over sodium sulphate. Solvent was removed to yield the product as a light brown solid (77 mg, 42%). NMR 1.50 (m, 2H), 1.62 (d, 6H), 1.68 (m, 2H), 2.32 (m, 1H), 2.59 (m, 6H), 2.80 (m, 2H), 4.12 (m, 2H), 5.09 (sept, 1H), 7.17 (m, 2H), 7.41 (m, 2H), 7.67 (d, 1H), 8.17 (m, 2H); m/z 407.29.

### Example 107

### 3-(3-Carboxypyrrolidin-1-ylcarbonylamino)-2,4-dimethyl-9-isopropyl-9H-carbazole

3-(3-Methoxycarbonylpyrrolidin-1-ylcarbonylamino)-2,4-dimethyl-9-isopropyl-9H-carbazole (Example 103) was hydrolysed by the procedure of Example 104. NMR 1.62 (d, 6H), 2.0-2.25 (m, 3H), 2.36 (s, 3H), 2.65 (s, 3H), 3.05-3.22 (m, 1H), 3.26-3.72 (m, 5H), 5.10 (sept, 1H), 7.16 (t, 1H), 7.3-7.5 (m, 2H), 7.62 (s, 1H), 7.66 (d, 1H), 8.15 (d, 1H); m/z 394.24.

### Example 108

### 3-[3-(N,N-Dimethylcarbamoyl)pyrrolidin-1-ylcarbonylamino]-2,4-dimethyl-9-isopropyl-9H-carbazole

3-(3-Carboxypyrrolidin-1-ylcarbonylamino)-2,4-dimethyl-9-isopropyl-9H-carbazole (Example 107) was coupled with dimethylamine by the procedure of Example 105. NMR (CDCl₃) 1.70 (d, 6H), 2.10-2.22 (m, 1H), 2.25-2.42 (m, 1H), 2.46 (s, 3H), 2.80 (s, 3H), 3.0 (s, 3H), 3.10 (s, 3H), 3.25-3.42 (m, 1H), 3.47-3.6 (m, 1H), 3.66-3.87 (m, 3H), 5.0 (sept, 1H), 5.72 (s, 1H), 7.19 (t, 1H), 7.23 (s, 1H), 7.4 (t, 1H), 7.5 (d, 1H), 8.2 (d, 1H); m/z 421.24.

### Example 109

### 3-(Piperidin-4-ylcarbonylamino)-2,4-dimethyl-9-isopropyl-9H-carbazole

3-Amino-2,4-dimethyl-9-isopropylcarbazole (Method 14; 1.44 g, 5.73 mmol) was stirred in dry THF (30 ml) with EDAC (1.208, 6.30 mmol), 4-dimethylaminopyridine (70 mg, 0.57 mmol) and 1-t-butyl-oxycarbonyl-piperdine-4-carboxylic acid (1.312 g, 5.73 mmol) at room temperature for 19 h. The mixture was extracted with DCM and washed with water before the organics were dried and the solvent removed. The residue was dissolved in DCM (30 ml) and trifluoroacetic acid (6 ml) was added. The reaction mixture was stirred at room temperature for 3.5 h and then basified with saturated potassium carbonate solution. Extraction with EtOAc then washing with water followed by drying over sodium sulphate and solvent removal yielded the title compound as a brown gum (1.599 g, 77%). M/z 364.44.

### Examples 110-112

Using the procedure of Example 109, the following compounds were prepared. For Example 111 the hydrolysis of the t-butyl-oxycarbonyl step was omitted

| Ex | Compound | NMR (CDCl₃) | MS |
|---|---|---|---|
| 110 | 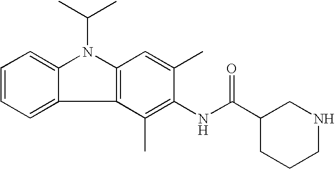 | 1.60 (d, 6H), 1.88 (m, 4H), 2.35 (s, 3H), 2.57 (m, 1H), 2.64 (s, 3H), 2.72 (m, 1H), 2.95 (m, 2H), 3.25 (m, 1H), 4.89 (sept, 1H), 7.11 (t, 1H), 7.17 (s, 1H), 7.30 (t, 1H), 7.42 (d, 1H), 8.09 (d, 1H), 9.13 (brs, 1H) | 364.33 |
| 111 | 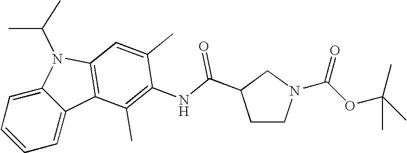 | 1.42 (s, 9H), 1.62 (d, 6H), 2.03-2.25 (m, 2H), 2.32 (s, 3H), 3.60 (s, 3H), 3.30 (s, 3H), 3.37-3.65 (m, 3H), 5.11 (sept, 1H), 7.18 (t, 1H), 7.35-7.48 (m, 2H), 7.70 (d, 1H), 8.15 (d, 1H), 9.40 (s, 1H) | 448.31 |
| 112 | 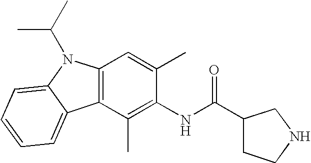 | 1.61 (d, 6H), 2.00-2.25 (m, 2H), 2.35 (s, 3H), 2.56-2.76 (m, 4H), 2.86-2.94 (m, 1H), 2.96-3.10 (m, 2H), 3.12-3.25 (m, 1H), 3.30-3.40 (m, 1H), 4.90 (sept, 1H), 7.05-7.55 (m, 4H), 7.85 (s, 1H), 8.08 (m, 1H) | 350.41 |

### Example 113

### 3-(1-Carbamoylpiperidin-4-ylcarbonylamino)-2,4-dimethyl-9-isopropyl-9H-carbazole

3-(Piperidin-4-ylcarbonylamino)-2,4-dimethyl-9-isopropyl-9H-carbazole (Example 109; 647 mg, 1.82 mmol) was stirred in water/ethanol (30 ml/30 ml) with potassium cyanate (534 mg, 6.594 mmol) at reflux for 16 h. The reaction mixture was cooled and diluted with water, then 2M HCl solution. The aqueous mixture was extracted with DCM and EtOAc, then the organics were combined and dried over sodium sulphate before solvent removal. Chromatography (eluent—EtOAc to EtOAc/MeOH 7:3) yielded the title compound as a brown gum (255 mg, 35%). NMR (CDCl₃) 1.58 (d, 6H), 1.74 (m, 6H), 2.22 (s, 3H), 2.41 (m, 1H), 2.50 (s, 3H), 2.76 (m, 2H), 3.90 (m, 1H), 4.72 (m, 1H), 4.86 (sept, 1H), 7.09 (m, 3H), 7.28 (m, 1H), 7.41 (m, 1H), 8.00 (m, 1H); m/z 405.40.

### Examples 114-115

Using the procedure of Example 113 the following compounds were prepared.

| Ex | Compound | NMR (CDCl₃) | MS | SM |
|---|---|---|---|---|
| 114 | 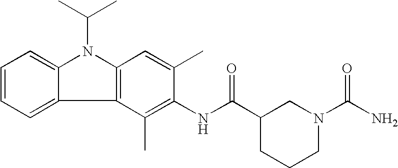 | 1.62 (d, 6H), 1.69 (m, 3H), 2.05 (m, 2H), 2.34 (s, 3H), 2.63 (s, 3H), 2.79 (d, 3H), 3.35 (m, 2H), 3.70 (m, 2H), 4.60 (br s, 2H), 4.91 (sept, 1H), 7.12 (t, 1H), 7.19 (s, 1H), 7.32 (t, 1H), 7.45 (d, 1H), 7.50 (s, 1H), 8.10 (d, 1H) | 407.30 | Ex 110 |
| 115 | 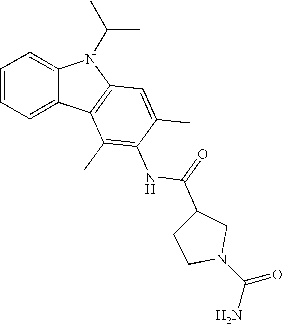 | 1.68 (d, 6H), 2.12-2.30 (m, 1H), 2.35 (s, 3H), 2.38-2.48 (m, 1H), 3.62 (s, 3H), 3.00-3.18 (m, 1H), 3.25-3.40 (m, 1H), 3.45-3.61 (m, 1H), 3.62-3.78 (m, 1H), 4.72 (s br, 1H), 4.85 (s br, 2H), 4.97 (sept, 1H), 7.08-7.60 (m, 4H), 8.15 (d, 1H) | 391.29 | Ex 112 |

### Example 116

### 3-[1-(N,N-Dimethylcarbamoyl)piperidin-4-ylcarbonylamino]-2,4-dimethyl-9-isopropyl-9H-carbazole

3-(Piperidin-4-ylcarbonylamino)-2,4-dimethyl-9-isopropyl-9H-carbazole (Example 109; 411 mg, 1.132 mmol) and pyridine (1.6 ml) were stirred in DCM (12 ml). Dimethylcarbamoylchloride (125 μl, 1.358 mmol) was added to the reaction mixture and allowed to stir for 16 h. 2M HCl solution was added and the mixture was extracted with DCM/MeOH (19:1). The organics were dried with sodium sulphate and the solvent was removed. Chromatography (eluent—EtOAc to EtOAc/MeOH 9:1) yielded the title compound as a yellow solid (115 mg, 23%). NMR (CDCl₃) 1.68 (d, 6H), 1.74 (m, 1H), 1.95 (m, 2H), 2.07 (m, 2H), 2.22 (s, 3H), 2.41 (s, 3H), 2.72 (s, 3H), 2.82 (m, 2H), 2.87 (s, 6H), 3.78 (m, 2H), 4.96 (sept, 1H), 6.84 (br s, 3H), 7.19 (t, 1H), 7.27 (s, 1H), 7.41 (t, 1H), 7.54 (d, 1H), 8.17 (d, 1H); m/z 435.30.

### Examples 117-118

Using the procedure of Example 116 the following compounds were prepared.

| Ex | Compound | NMR (CDCl₃) | MS | SM |
|---|---|---|---|---|
| 117 | 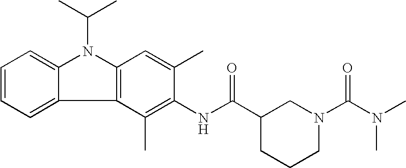 | 1.71 (d, 6H), 1.75 (m, 3H), 2.03 (m, 1H), 2.18 (m, 1H), 2.43 (s, 3H), 2.73 (s, 3H), 2.86 (s, 6H), 3.23 (m, 1H), 3.50 (m, 1H), 3.64 (m, 1H), 3.74 (m, 1H), 4.98 (sept, 1H), 7.20 (t, 1H), 7.26 (s, 1H), 7.42 (t, 1H), 7.53 (d, 1H), 8.20 (m, 2H) | 435.44 | Ex 110 |
| 118 | 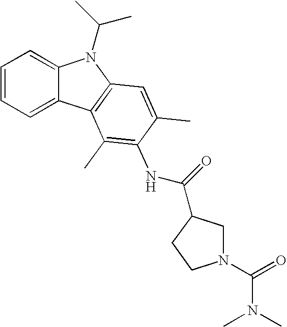 | 1.60-1.78 (m, 9H), 2.25-2.33 (m, 2H), 2.35-2.48 (m, 4H), 2.65-2.85 (m, 5H), 3.06 (s, 1H), 3.15 (s, 1H), 3.50-3.60 (m, 2H), 3.65-3.75 (m, 1H), 3.85-3.95 (m, 1H), 4.95 (sept, 1H), 7.16-7.62 (m, 4H), 8.18 (d, 1H) | 421.31 | Ex 112 |

### Example 119

### 3-[1-(N-Methylcarbamoyl)piperidin-3-ylcarbonylamino]-2,4-dimethyl-9-isopropyl-9H-carbazole

Phosgene (0.8 ml, 1.55 mmol, 20% soln. in toluene) was stirred in dry THF (3 ml) under argon and cooled in an ice bath. A solution of N-(3-carboxypiperidine)-3-amino-2,4-dimethyl-9-isopropylcarbazole (Example 110; 511 mg, 1.41 mmol) in dry THF (3 ml) was added dropwise followed by triethylamine (0.2 ml, 1.1 mmol). After stirring for 1 h, methylamine (1.41 ml, 2.82 mmol, 2M soln. in THF) was added and the reaction mixture was allowed to warm to room temperature and stirred for 16 h. The reaction mixture was extracted with DCM and washed with saturated potassium carbonate solution and water. The organics were dried over sodium sulphate and solvent removed. Chromatography on biotage system (eluent—DCM to DCM/MeOH 19:1) yielded the title compound as a white solid (112 mg, 19%). NMR (CDCl₃) 1.62 (m, 3H), 1.70 (d, 6H), 2.00 (m, 1H), 2.14 (m, 1H), 2.38 (s, 3H), 2.70 (s, 3H), 2.79 (d, 3H), 3.29 (m, 1H), 3.44 (m, 1H), 3.75 (d, 2H), 4.80 (m, 1H), 4.96 (sept, 1H), 7.19 (t, 1H), 7.25 (s, 1H), 7.40 (t, 1H), 7.53 (d, 1H), 7.73 (s, 1H), 8.16 (d, 1H); m/z 421.29.

### Example 120

Using the procedure of Example 119 the following compound was prepared.

| Ex | Compound | NMR (CDCl₃) | MS | SM |
|---|---|---|---|---|
| 120 | 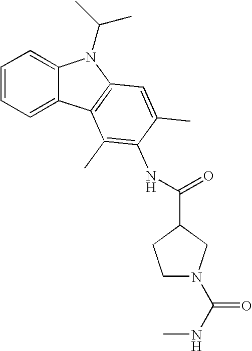 | 1.65-1.75 (m, 7H), 2.38-2.46 (m, 6H), 2.70 (s, 3H), 2.72-2.78 (m, 1H), 2.80-2.85 (m, 4H), 3.12-3.26 (m, 1H), 3.57-3.85 (m, 1H), 3.70-3.80 (m, 2H), 4.95 (sept, 1H), 7.05 (s, 1H), 7.20 (t, 1H), 7.40 (t, 1H), 8.18 (d, 1H) | 407.27 | Ex 112 |

### Example 121

### 3-(1-Acetylpyrrolidin-3-ylcarbonylamino)-2,4-dimethyl-9-isopropyl-9H-carbazole

Acetic anhydride (239 mg, 2.34 mmol) was added to a stirring solution of 3-[1-(t-butoxycarbonyl)pyrrolidin-3-ylcarbonylamino]-2,4-dimethyl-9-isopropyl-9H-carbazole (Example 111; 500 mg) in glacial acetic acid (2.2 ml). The mixture was heated to 120° C. for 5 hours. The mixture was concentrated in vacuo and the residue was chromatographed (eluent i) ethyl acetate ii) 5% methanol/dichloromethane) to give the title compound (150 mg, 35%). NMR (CDCl₃) 1.60-1.75 (m, 6H), 2.08-2.22 (m, 1H), 2.28-2.38 (m, 3H), 2.39-2.48 (m, 1H), 2.52-2.62 (m, 3H), 3.70-3.80 (m, 1H), 2.92-3.18 (m, 1H), 3.25-3.95 (m, 4H), 4.85-5.05 (m, 1H), 7.06-7.6 (m, 4H), 8.02-8.15 (m, 1H); m/z 390.30.

### Example 122

### 3-(1-Acetylpiperidin-4-ylcarbonylamino)-2,4-dimethyl-9-isopropyl-9H-carbazole

3-Amino-2,4-dimethyl-9-isopropylcarbazole (Method 14; 250 mg, 0.99 mmol) was dissolved in dry DMF (15 ml) and stirred for 10 minutes at room temperature. To this were added o-(7-azabenzotriazole-1-yl)-N,N,N′,N′-tetramethyluronium hexafluorophosphate, (437 mg, 0.987 mmol), N,N-diisopropylethylamine (255 mg, 1.974 mmol) and 1-acetylpiperidine-4-carboxylic acid (169 mg, 0.987 mmol) and the reaction mixture was stirred at room temperature for 6 hours. This was then concentrated in vacuo to which was added water (150 ml). This was extracted with chloroform (3×100 ml), dried and concentrated in vacuo to yield a light brown solid (348 mg, 87%). NMR 1.43-1.77 (m, 8H), 1.80-1.98 (m, 2H), 2.02 (s, 3H), 2.30 (s, 3H), 2.58 (s, 3H), 3.03-3.19 (m, 1H), 3.82-3.95 (m, 1H), 4.35-4.45 (m, 1H), 5.10 (sept, 1H), 7.15 (t, 1H), 7.32-7.43 (m, 2H), 7.66 (d, 1H), 8.12 (d, 1), 9.20 (s, 1H); m/z 406.30.

### Example 123

Using the procedure of Example 122 the following compound was prepared.

| Ex | Compound | NMR (CDCl₃) | MS | SM |
|---|---|---|---|---|
| 123 | 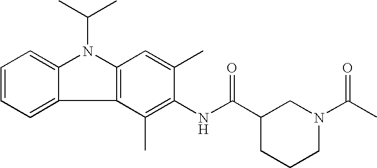 | 1.52 (m, 1H), 1.65 (d, 6H), 406.1 1.82 (m, 2H), 2.07 (s, 3H), 2.13 (m, 1H), 2.36 (s, 3H), 2.66 (s, 3H), 2.96 (m, 1H), 3.15 (m, 1H), 3.96 (m, 1H), 4.24 (m, 1H), 5.07 (sept, 1H), 7.16 (t, 1H), 7.40 (m, 2H), 7.62 (d, 1H), 8.16 (d, 1H), 8.85 (brs, 2H) | 406.1 | Method 14 |

### Examples 124-164

o-(7-Azabenzotriazole-1-yl)-N,N,N′,N′-tetramethyluronium hexafluorophosphate, (6.34 g, 16.67 mmol) and N,N-diisopropylethylamine (5.3 ml, 30.43 mmol) were dissolved in DMF (95.4 ml) to give solution A. 3-Amino-4-methyl-9-isopropylcarbazole (Method 21; 5.5 g, 23.11 mmol) was dissolved in DMF (40 ml) to give solution B. Solution A (2.65 ml) was added to the appropriate acid and the mixture was stirred at room temperature for 15 minutes. To this was added solution B (0.74 ml) and the reaction mixture was stirred at room temperature for 40 hours. 1 ml of each reaction was taken and purified using a preparative LC/MS-HPLC instrument to give the required compounds.

In Examples 150-164 an equivalent quantity of 3-amino-2,4 dimethyl-9-isopropylcarbazole (Method 14 ) was used in solution B.

| Ex | Compound | MS |
|---|---|---|
| 124 | 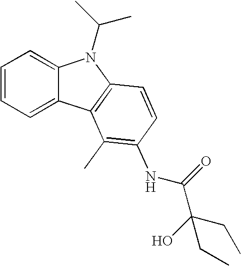 | 353 |
| 125 | 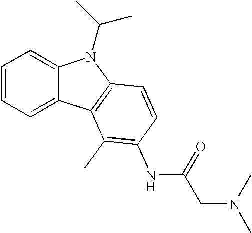 | 324 |
| 126 | 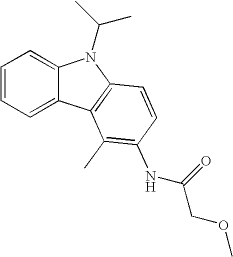 | 311 |
| 127 | 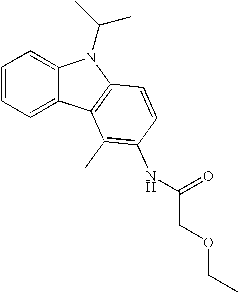 | 325 |
| 128 | 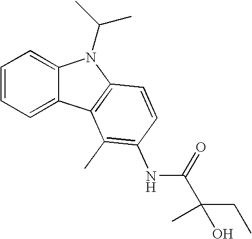 | 339 |
| 129 | 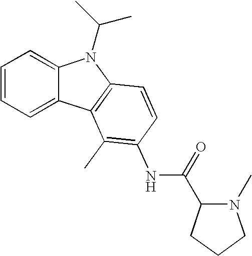 | 350 |
| 130 | 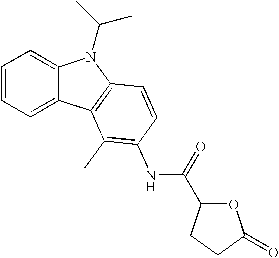 | 351 |
| 131 | 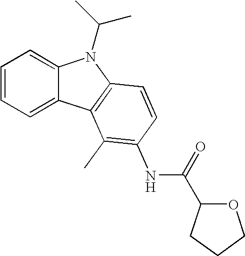 | 337 |
| 132 | 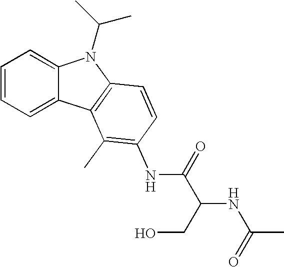 | 366 |
| 133 | 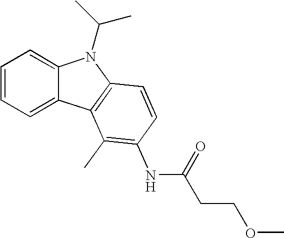 | 325 |
| 134 | 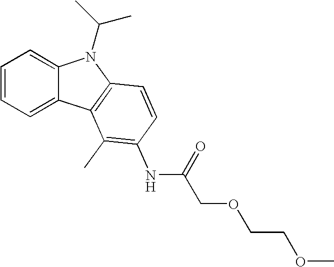 | 355 |
| 135 | 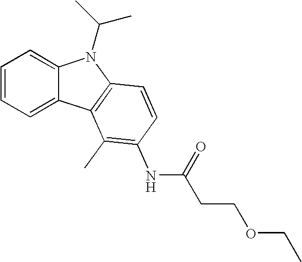 | 339 |
| 136 | 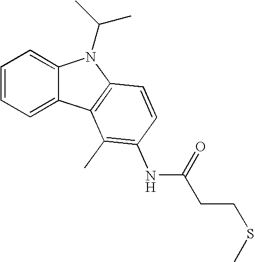 | 341 |
| 137 | 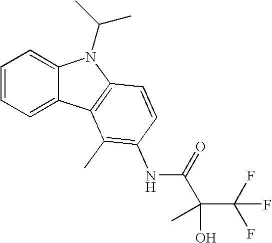 | 379 |
| 138 | 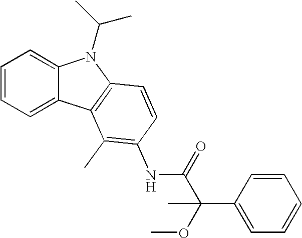 | 401 |
| 139 | 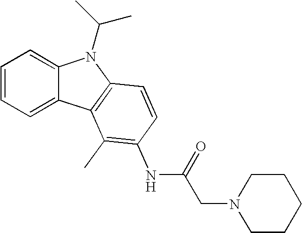 | 364 |
| 140 | 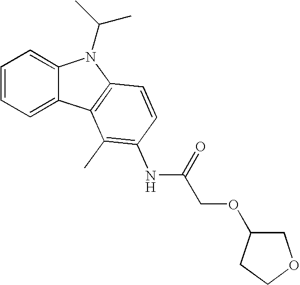 | 367 |
| 141 | 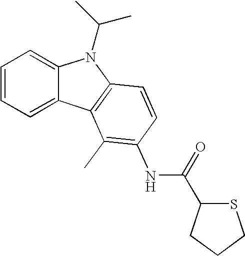 | 353 |
| 142 | 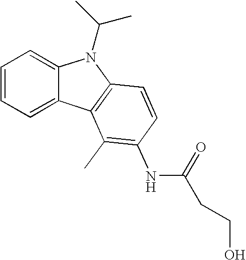 | 311 |
| 143 | 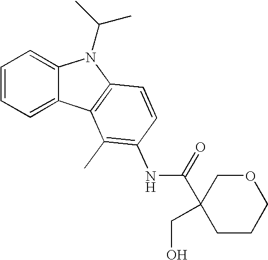 | 381 |
| 144 | 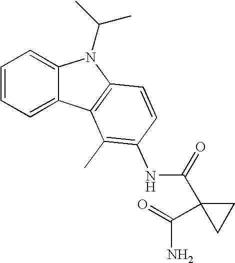 | 350 |
| 145 | 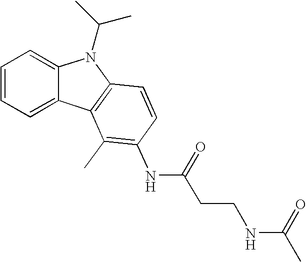 | 352 |
| 146 | (Acid ref. Monatsch Chem. 1990, 121 (11), 963-70) | 349 |
| | 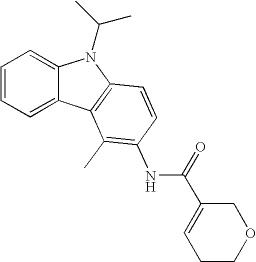 | |
| 147 | (Acid ref. Synthesis 1986, (12), 1070-4) | 369 |
| | 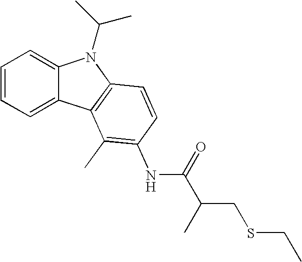 | |
| 148 | (Acid ref. J. Bioactiv. Comptat. Polym. 1999, | 349 |
| | 5(4), 420-429) | |
| | 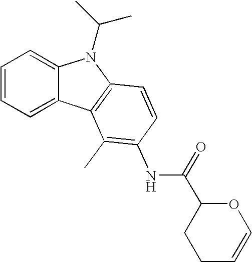 | |
| 149 | 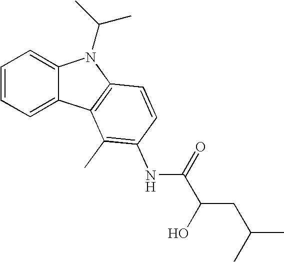 | 353 |
| 150 | 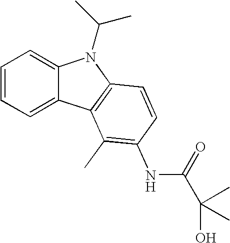 | 324 |
| 151 | 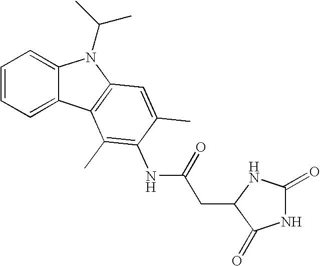 | 393 |
| 152 | 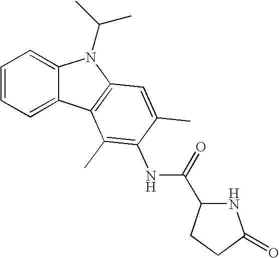 | 364 |
| 153 | 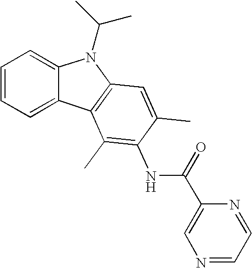 | 359 |
| 154 | 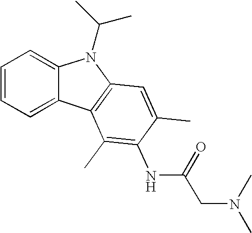 | 338 |
| 155 | 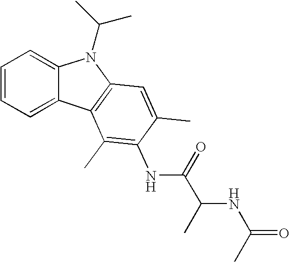 | 366 |
| 156 | 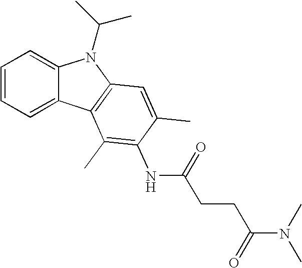 | 380 |
| 157 | 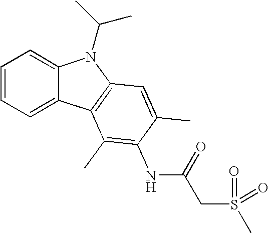 | 373 |
| 158 | 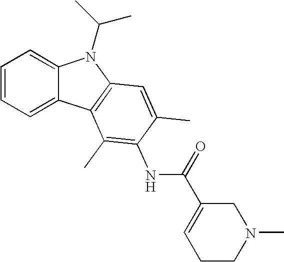 | 376 |
| 159 | 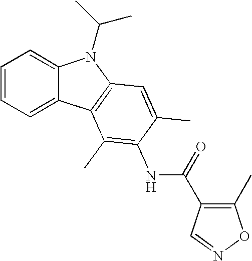 | 362 |
| 160 | 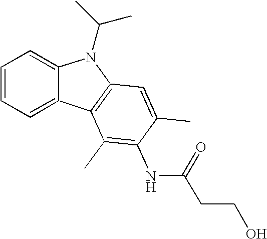 | 325 |
| 161 | 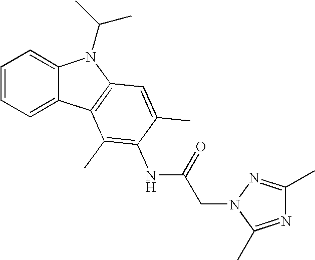 | 390 |
| 162 | 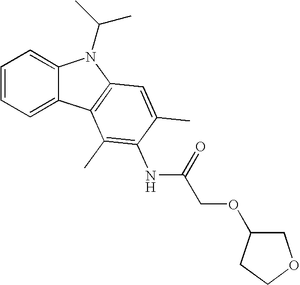 | 381 |
| 163 | 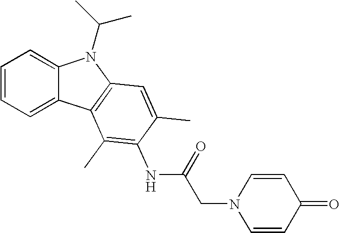 | 388 |
| 164 | (Acid ref. WO 9414776) | 387 |
| | 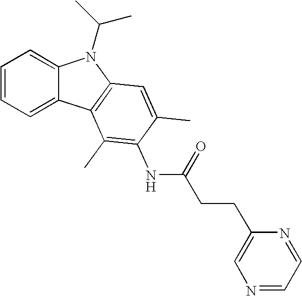 | |

### Example 165

### 5-Methyl-6-morpholinocarbonylamino-9-isopropyl-9H-1,2,3,4-tetrahydrocarbazole

The title compound was prepared from 9-isopropyl-1,2,3,4-tetrahydro-5-methyl-6-aminocarbazole (Method 53) in a similar manner as described for Example 1. NMR (300 MHz); 7.94 (s, 1H), 7.17 (d, 1H), 6.73 (d, 1H), 4.59 (m, 1H), 3.60 (m, 4H), 3.40 (m, 4H), 2.92 (m, 2H), 2.72 (m, 2H), 2.34 (s, 3H), 1.75 (m, 4H), 1.48 (d, 6H); m/z 356.5.

### Example 166

### 3-(3-Carboxypiperidin-1-ylcarbonylamino)-2,4-dimethyl-9-isopropyl-9H-carbazole

3-[3-(Ethoxycarbonyl)piperidin-1-ylcarbonylamino]-2,4-dimethyl-9-isopropyl-9H-carbazole (Example 100) was hydrolysed in a similar method as described for Example 104 to give the title compound. NMR (300 MHz; CDCl₃) 1.3-1.85 (m, 4H), 1.60 (d, 6H), 1.80 (m, 3H), 2.29 (s, 3H), 2.4 (1H, m), 2.62 (s, 3H), 3.1-3.7 (m, 4H), 4.90 (sep, 1H), 5.3 (1H, brs), 6.00 (brs, 1H), 7.10 (t, 1H), 7.18 (s, 1H), 7.30 (t, 1H), 7.42 (d, 1H), 8.05 (d, 1H); m/z 408.24.

### Example 167

### 3-[3-(N,N-Dimethylcarbamoyl)piperidin-1-ylcarbonylamino]-2,4-dimethyl-9-isopropyl-9H-carbazole

The title compound was prepared from 3-(3-carboxypiperidin-1-ylcarbonylamino)-2,4-dimethyl-9-isopropyl-9H-carbazole (Example 166) in a similar method as described for Example 105. NMR (300 MHz; CDCl₃) 1.62 (d, 6H), 1.80 (m, 3H), 2.38 (s, 3H), 2.69 (s, 3H), 2.73-3.05 (m, 4H), 2.88 (s, 3H), 3.01 (s, 3H), 3.87 (m, 1H), 4.15 (m, 1H), 4.90 (sep, 1H), 6.00 (brs, 1H), 7.10 (t, 1H), 7.16 (s, 1H), 7.32 (t, 1H), 7.44 (d, 1H), 8.10 (d, 1H); m/z 435.40.

### Example 168

### 3-[3-(N-Methylcarbamoyl)piperidin-1-ylcarbonylamino]-2,4-dimethyl-9-isopropyl-9H-carbazole

The title compound was prepared from 3-(3-carboxypiperidin-1-ylcarbonylamino)-2,4-dimethyl-9-isopropyl-9H-carbazole (Example 166) in a similar method as described for Example 105 except using methylamine. NMR (300 MHz; CDCl₃) 1.63 (d, 6H), 1.65 (m, 3H), 2.03 (m, 1H), 2.30 (m, 1H), 2.36 (s, 3H), 2.64 (s, 3H), 2.66 (s, 3H), 3.40 (m, 2H), 3.52 (m, 1H), 3.73 (m, 1H), 4.90 (sep, 1H), 6.10 (brs, 1H), 6.63 (brs, 1H), 7.13 (t, 1H), 7.19 (s, 1H), 7.34 (t, 1H), 7.46 (d, 1H), 8.09 (d, 1H); m/z 421.30.

Preparation of Starting Materials

The starting materials for the Examples above are either commercially available or are readily prepared by standard methods from known materials. For example the following reactions are illustrations but not limitations of the preparation of some of the starting materials used in the above reactions.

Method 1

1,2,3,4-Tetrahydro-5,7-dimethylcarbazole

A solution of 2-chlorocyclohexanone (6.9 ml, 60.4 mmol) in EtOH (40 ml), was slowly added to a refluxing solution of 3,5-dimethylaniline (15 ml, 120.8 mmol) in EtOH (40 ml). The reaction mixture was stirred at reflux for 20 h, concentrated and dissolved in diethyl ether/water (50/50). The aqueous layer was washed with diethyl ether and the combined organic layers were washed with 5M hydrochloric acid, water and sodium bicarbonate solution respectively. Drying and solvent removal gave the title compound as a brown solid. Rf (diethyl ether) 0.82; NMR 10.31 (s, 1H), 6.80 (s, 1H), 6.44 (s, 1H), 2.88 (m, 2H), 2.63 (m, 2H), 2.50 (s, 3H), 2.28 (s, 3H), 1.77 (m, 4H).

Method 2

1,2,3,4-Tetrahydro-5,7-dimethyl-6-nitrocarbazole

To a stirred solution of 1,2,3,4-tetrahydro-5,7-dimethylcarbazole (Method 1; 8.24 g, 41.4 mmol) in concentrated sulphuric acid (100 ml) at 0° C. was added potassium nitrate (4.19 g, 41.4 mmol) in one portion. The reaction mixture was stirred for 2 h before being poured onto ice/water. The yellow precipitate was filtered, washed with 10% aqueous ammonia, water and then dissolved in EtOAc. Drying then purification by chromatography (eluent—DCM) yielded the title compound as an orange solid. Yield 5.193 g (51%); Rf (DCM) 0.55; ¹H NMR 10.96 (s, 1H), 7.03 (s, 1H), 2.98 (m, 2H), 2.67 (m, 2H), 2.45 (s, 3H), 2.25 (s, 3H), 1.77 (m, 4H); m/z 243.63.

Methods 3-4

Following the procedure of Method 2 and using the appropriate starting materials the following compounds were made.

| Meth | Compound | NMR | SM |
|---|---|---|---|
| 3 | 6-Nitro-1-methyl- | (CDCl₃) 1.3(d, 3H), 1.6(m, 1H), | ¹ |
| | 1,2,3,4-tetrahydro- | 1.8(m, 1H), 2.0-2.1(m, 2H), 2.6- | |
| | carbazole | 2.8(m, 2H), 3.0(m, 1H), 7.3 | |
| | | (d, 1H), 8.0(d, 1H), 8.2(s, 1H), | |
| | | 8.4(s, 1H) | |
| 4 | 1,2,3,4-Tetrahydro-8- | NMR(CDCl₃) 8.18(brs, 1H), | Meth |
| | chloro-5-methyl-6- | 7.79(s, 1H), 3.00(m, 2H), | 23 |
| | nitrocarbazole | 2.84(s, 3H), 2.78(m, 2H), | |
| | | 1.90(m, 4H) | |

¹ 1-Methyl-1,2,3,4-tetrahydro-carbazole: Can. J. Chem vol 43 p300 1965

Method 5

2,4-Dimethyl-3-nitro-9H-carbazole

To a stirred solution of 1,2,3,4-tetrahydro-5,7-dimethyl-6-nitrocarbazole (Method 2; 14 g, 57.4 mmol) in 1,4-dioxane (200 ml) at room temperature was added DDQ (26 g, 114.8 mmol) portionwise. The reaction mixture was stirred at 100° C. for 4 h before being concentrated in vacuo. Chromatography (eluent—20% EtOAc/isohexane—50% EtOAc/isohexane) yielded the title compound as an orange solid. Yield 6.62 g (48%). Rf (20% EtOAc/isohexane) 0.24; NMR 11.75 (s, 1H), 8.17 (d, 1H), 7.56 (d, 1H), 7.46 (t, 1H), 7.34 (s, 1H), 7.24 (t, 1H), 2.74 (s, 3H), 2.42 (s, 3H); m/z 239.59.

Methods 6-7

Following the procedure of Method 5 and using the appropriate starting materials the following compounds were made.

| | | | |
|---|---|---|---|
| Meth | Compound | NMR | SM |

| | | | |
|---|---|---|---|
| 6 | 1-Chloro-9-isopropyl- | (CDCl₃) 8.34(d, 1H), 8.09(s, | Meth |
| | 4-methyl-3- | 1H), 7.80(d, 1H), | 11 |
| | nitrocarbazole | 7.54(t, 1H), 7.36(t, 1H), | |
| | | 6.32(sep, 1H), 3.10(s, 3H), | |
| | | 1.77(d, 6H) | |
| 7 | 3-Nitro-8-methyl-9H- | 2.6(s, 3H), 7.2(t, 1H), | Meth |
| | carbazole | 7.3(d, 1H), 7.6(d, 1H), | 3 |
| | | 8.2(d, 1H), 8.3(d, 1H), | |
| | | 9.1(s, 1H), 11.9(s, 1H) | |

Method 8

2,4-Dimethyl-9-isopropyl-3-nitro-9H-carbazole

2,4-Dimethyl-3-nitrocarbazole (Method 5; 6.62 g, 27.6 mmol) and caesium carbonate (18 g, 55.2 mmol) were stirred in dry DMF (100 ml) under an argon atmosphere at room temperature. 2-Iodopropane (5.51 ml, 55.2 mmol) was added to the reaction mixture before heating at 85° C. More caesium carbonate (9 g, 27.6 mmol) and 2-iodopropane (2.76 ml, 27.6 mmol) were added after 3.5 h and 5.5 h. The reaction solvent was removed in vacuo, the residue extracted into EtOAc and washed with water and brine. The organic layers were dried and solvent removed in vacuo. Chromatography (eluent—10% EtOAc/isohexane—20% EtOAc/isohexane) yielded the title compound as a yellow solid. Yield 6.18 g (79%). Rf (50% EtOAc/isohexane) 0.65; NMR 8.20 (d, 1H), 7.78 (d, 1H), 7.62 (brs, 1H), 7.48 (t, 1H), 7.25 (t, 1H), 5.17 (sep, 1H), 2.72 (s, 3H), 2.42 (s, 3H).

Methods 9-13

Following the procedure of Method 8 and using the appropriate starting materials the following compounds were made.

| Meth | Compound | NMR | M/z | SM |
|---|---|---|---|---|
| 9 | 1,2,3,4-Tetrahydro-5,7- | 7.83(s, 1H), 4.65(sep, 1H), 2.90(m, 2H), | 287.27 | Meth 2 |
| | dimethyl-6-nitro-9- | 2.73(m, 2H), 2.48(s, 3H), 2.30(s, | | |
| | isopropylcarbazole | 3H), 1.76(m, 4H), 1.48(d, 6H) | | |
| 10 | 3-Fluoro-9-isopropyl- | (CDCl₃) 1.7(d, 6H), 5.0(m, 1H), 7.1-7.2 | 227 | ¹ |
| | 9H-carbazole | (m, 2H), 7.4-7.5(m, 3H), 7.8(d, | (M⁺) | |
| | | 1H), 8.0(d, 1H) | | |

| | | | |
|---|---|---|---|
| 11 | 1,2,3,4-Tetrahydro-8- | (CDCl₃) 7.79(s, 1H), 6.15(sep, 1H), | Meth 4 |
| | chloro-9-isopropyl-5- | 3.07(m, 2H), 2.91(m, 2H), 2.84(s, | |
| | methyl-6-nitrocarbazole | 3H), 1.90(m, 4H), 1.55(d, 6H) | |
| 12 | 3-Nitro-8-methyl-9- | (CDCl₃) 1.6(t, 3H), 2.8(s, 3H), 4.6(q, | Meth 7 |
| | ethyl-carbazole | 2H), 7.2(m, 2H), 7.4(d, 1H), 8.0(d, | |
| | | 1H), 8.4(d, 1H), 9.0(s, 1H) | |
| 13 | 8-Methyl-3-nitro-6- | 1.8(d, 6H), 2.8(s, 3H), 5.5(m, 1H), 7.1 | Meth 35 |
| | fluoro-9-isopropyl-9H- | (m, 1H), 7.6(m, 2H), 8.3(d, 1H), 8.9(s, | |
| | carbazole | 1H) | |

¹ 3-Fluoro-9H-carbazole (Ref. Kapil et al J. Med. Chem 1973 v. 16 n. 4 p. 427)

Method 14

3-Amino-2,4-dimethyl-9-isopropyl-9H-carbazole

To a stirred solution of 2,4-dimethyl-9-isopropyl-3-nitrocarbazole (Method 8; 6.18 g, 21.9 mmol) in EtOAc (200 ml) under an argon atmosphere, was added 10% palladium on carbon (800 mg). The reaction mixture was stirred at room temperature under a hydrogen atmosphere. More 10% palladium on carbon (800 mg) was added after 64 h and 88 h. The catalyst was filtered off through diatomaceous earth and washed with EtOAc. The filtrate was concentrated, chromatography (eluent—20% EtOAc/isohexane—50% EtOAc/isohexane) yielded the title compound as a white/yellow solid 5.294 g (96%). Rf (20% EtOAc/isohexane) 0.19; NMR (CDCl₃) 8.24 (d, 1H), 7.48 (d, 1H), 7.36 (t, 1H), 7.16 (m, 2H), 4.95 (sep, 1H), 2.76 (brs, 3H), 2.42 (brs, 3H), 1.68 (d, 6H); m/z 253.74.

Methods 15-19

Following the procedure of Method 14 and using the appropriate starting materials the following compounds were made.

| Meth | Compound | NMR | M/z | SM |
|---|---|---|---|---|
| 15 | 1,2,3,4-Tetrahydro-5,7- | 7.01(s, 1H), 4.50(sept, 1H), 3.32(brs, 2H), | 257.73 | Meth 9 |
| | dimethyl-6-amino-9- | 3.00(m, 2H), 2.68(m, 2H), 2.47 | | |
| | isopropylcarbazole | (s, 3H), 2.30(s, 3H), 1.84(m, 4H), 1.53 | | |
| | | (d, 6H) | | |
| 16 | 3-Amino-4,8-dimethyl- | (CDCl₃) 1.6(t, 3H), 2.7(s, 3H), 2.8(s, | 239 | Meth 28 |
| | 9-ethyl-carbazole | 3H), 4.5(q, 2H), 7.0(d, 1H), 7.1-7.2 | | |
| | | (m, 3H), 8.1(d, 1H) | | |
| 17 | 3-Amino-4-methyl-6- | (CDCl₃) 1.6(d, 6H), 2.3(s, 3H), 4.9(m, | 257 | Meth 27 |
| | fluoro-9-isopropyl-9H- | 1H), 6.9(d, 1H), 7.1-7.2(m, 2H), 7.4(d, | | |
| | carbazole | 1H), 7.9(d, 1H) | | |
| 18 | 3-Amino-4,8-dimethyl- | 1.6(d, 6H), 2.7(s, 3H), 2.8(s, 3H), 5.4 | 271 | Meth 30 |
| | 6-fluoro-9-isopropyl- | (m, 1H), 6.9(d, 2H), 7.2(d, 1H), 7.8(d, | | |
| | 9H-carbazole | 1H) | | |

| | | | |
|---|---|---|---|
| 19 | 3-Amino-9-(2- | 1.55(d, 3H), 2.55(s, 3H), 3.3(s, 3H), 3.75(m, | Meth 38 |
| | methoxy-1- | 1H), 3.95(m, 1H), 4.55(brs, 2H), 5.0(m, 1H), | |
| | methylethyl)-4-methyl- | 6.9(d, 1H), 7.1(t, 1H), 7.25(dd, 2H), 7.55(d, | |
| | carbazole | 1H), 8.15(d, 1H) | |

Method 20

3-Amino-4,9-diisopropyl-9H-carbazole

3-Nitro-4,9-diisopropyl-9H-carbazole (Method 25; 0.55 g, 1.86 mM) in EtOH (20 ml) was hydrogenated over 10% palladium on carbon (100 mg) at ambient temperature under atmospheric pressure of hydrogen. The catalyst was filtered off through diatomaceous earth and the filtrate was concentrated. Chromatography (eluent gradient of hexane to DCM) gave the title compound as a pale brown foam 0.55 g. Rf (Et₂O) 0.41; NMR (CDCl₃) 8.29 (d, 1H), 7.53 (d, 1H), 7.41 (t, 1H), 7.27 (d, 1H), 7.16 (t, 1H), 6.90 (d, 1H), 4.98 (sep, 1H), 4.43 (br m, 1H), 1.71 (d, 6), 1.63 (d, 6H); m/z 267.14.

Method 21

Following the procedure of Method 20 and using the appropriate starting materials but purifying the product by chromatography (eluent—50% EtOAc/isohexane) following compounds were made.

| Meth | Compound | NMR | M/z | SM |
|---|---|---|---|---|
| 21 | 9-Isopropyl-4- | 8.15(d, 1H), 7.53(d, 1H), 7.28 | 239.71 | Meth |
| | methyl-3-amino- | (m, 2H), 7.05(t, 1H), | | 26 |
| | 9H-carbazole | 6.85(d, 1H), 4.97(sep, 1H), | | |
| | | 4.50(brs, 2H), 2.55(s, 3H), | | |
| | | 1.56(d, 6H) | | |

Method 22

Benzophenone (2-chloro-5-methylphenyl)hydrazone

3-Bromo-4-chlorotoluene (6.45 ml, 48.7 mmol), benzophenone hydrazone (9.54 g, 48.7 mmol), palladium acetate (112 mg, 0.5 mmol) and (S)-(-)-2,2′-bis(diphenyl-phosphino)-1,1′-binaphthyl (311 mg, 0.5 mmol) were stirred together in dry toluene (25 ml) under argon at room temperature. Sodium t-butoxide (6.55 g, 68.2 mmol) was added in one portion followed by more dry toluene (25 ml). The reaction mixture was heated at 80° C. for 24 h before cooling to room temperature then dilution with diethyl ether. The precipitate was filtered off, washed with diethyl ether then the filtrate solvent was removed in vacuo. Chromatography (eluent—10% EtOAc/isohexane) yielded the title compound as an orange/yellow solid. Rf (10% EtOAc/isohexane) 0.53; NMR 7.89 (s, 1H), 7.68-7.09 (m, 12H), 6.63 (dd, 1H), 2.29 (s, 3H); m/z 321.44.

Method 23

1,2,3,4-Tetrahydro-8-chloro-5-methylcarbazole

Benzophenone (2-chloro-5-methylphenyl)hydrazone (Method 22; 48.7 mmol), p-toluenesulphonic acid monohydrate (19 g, 0.1 mmol) and cyclohexanone (7.55 ml, 73 mmol) were stirred in EtOH (250 ml) at reflux for 40 h. After removal of EtOH in vacuo the residue was diluted with EtOAc, washed with saturated sodium bicarbonate solution, water and brine respectively and then dried over potassium carbonate. Solvent was removed in vacuo and chromatography (eluent—isohexane—20% DCM/isohexane) yielded the title compound as an oil 8.19 g (77%). Rf (20% DCM/isohexane) 0.39; NMR 10.80 (brs, 1H), 6.85 (d, 1H), 6.62 (d, 1H), 2.86 (m, 2H), 2.69 (m, 2H), 2.51 (s, 3H), 1.75 (m, 4H).

Method 24

3-Amino-1-chloro-4-methyl-9-isopropylcarbazole Hydrochloride Salt

Sodium hydrosulphite (428 mg, 2.46 mmol) was added to a stirred solution of 1-chloro-9-isopropyl-4-methyl-3-nitrocarbazole (Method 6; 123 mg, 0.41 mmol) in EtOH (10 ml) and water (5 ml). The reaction mixture was heated at reflux for 2 h. After removal of the EtOH in vacuo the residual liquid was extracted with EtOAc, washed with water and dried. Solvent removal in vacuo followed by chromatography (eluent—20% EtOAc/isohexane) yielded a gum. The title compound was isolated by treatment with hydrogen chloride (2M solution in diethyl ether) followed by filtration. Rf (50% EtOAc/isohexane) 0.47; NMR 10.15 (brs, 2H), 8.30 (d, 1H), 7.90 (d, 1H), 7.58 (s, 1H), 7.55 (t, 1H), 7.30 (t, 1H), 6.10 (sep, 1H), 2.75 (s, 3H), 1.69 (d, 6H); m/z 273.67.

Method 25

3-Nitro-4,9-diisopropyl-9H-carbazole

Isopropylmagnesium chloride (2M solution in tetrahydrofuran) (2.23 ml, 4.46 mM) was added to 3-nitro-9-isopropyl-9H-carbazole (Method 31; 1.13 g, 4.45 mM) in tetrahydrofuran (20 ml) at −15° C. After 1 hour DDQ (2.22 g, 9.79 mM) was added and then allowed to warm to ambient temperature for 18 hours. The mixture was diluted with DCM, washed with aqueous potassium carbonate solution, dried over sodium sulphate and concentrated. Chromatography (eluent gradient of hexane to DCM) gave the title compound as a yellow solid 0.56 g (46%). Rf (DCM:hexane (2:3)) 0.29; NMR (CDCl₃) 8.20 (d, 1H), 7.67 (d, 1H), 7.57 (d, 1H), 7.45 (t, 1H), 7.32 (d, 1H), 7.24 (t, 1H), 4.97 (sep, 1H), 4.16 (sep, 1H), 1.66 (d, 6H), 1.56 (d, 6H); m/z 297.18.

Methods 26-30

Following the procedure of Method 25 and using the appropriate starting materials the following compounds were made.

| Meth | Compound | NMR | M/z | SM |
|---|---|---|---|---|
| 26 | 9-Isopropyl-4- | 8.32(d, 1H), 8.04(d, 1H), 7.87(d, 1H), 7.73(d, | 269.1 | Meth 31 |
| | methyl-3-nitro-9H- | 1H), 7.53(t, 1H), 7.30(t, 1H), 5.22 | | |
| | carbazole | (sep, 1H), 3.02(s, 3H), 1.65(d, 6H) | | |
| 27 | 3-Nitro-4-methyl-6- | (CDCl₃) 1.7(d, 6H), 3.1(s, 3H), 5.0(m, | 287 | Meth 34 |
| | fluoro-9-isopropyl- | 1H), 7.2(d, 1H), 7.4(d, 1H), 7.5(d, 1H), | | |
| | 9H-carbazole | 8.0(d, 1H), 8.1(d, 1H) | | |
| 28 | 3-Nitro-4,8- | (CDCl₃) 1.4(t, 3H), 2.8(s, 3H), 3.1(s, 3H), | 269 | Meth 12 |
| | dimethyl-9-ethyl- | 4.6(q, 2H), 7.2-7.3(m, 3H), 8.1(d, 1H), 8.2 | | |
| | carbazole | (d, 1H) | | |

| | | | |
|---|---|---|---|
| 29 | 3-Nitro-4-methyl- | 11.88(brs, 1H), 8.29(d, 1H), 8.02(d, 1H), 7.60(m, | |
| | 9H-carbazole | 1H), 7.48(m, 2H), 7.28(m, 1H), 3.02(s, 3H) | |
| 30 | 3-Nitro-4,8- | 1.7(d, 6H), 2.8(s, 3H), 3.0(s, 3H), 5.5(m, 1H), 7.0 | Meth 13 |
| | dimethyl-6-fluoro-9- | (d, 1H), 7.5(d, 1H), 7.8(d, 1H), 8.0(d, 1H): m/z 301 | |
| | isopropyl-9H- | | |
| | carbazole | | |

Method 31

3-Nitro-9-isopropyl-9H-carbazole

3-Nitro-9H-carbazole (Synth. Commun., 24 (1994), 1; 0.99 g, 4.68 mM) was added to NaH (60% in oil; 281 mg, 7.02 mM) in DMF (35 ml) followed by isopropyl bromide (0.66 ml, 7.02 nM) at ambient temperature under inert atmosphere and heated to 60° C. for 18 hours. On cooling to room temperature the mixture was diluted with DCM, washed with water, dried over sodium sulphate and concentrated. Chromatography (eluent gradient of hexane to DCM) gave the title compound 0.98 g (79%); Rf (70% DCM/isohexane) 0.24; NMR (CDCl₃) 9.16 (d, 1H), 8.40 (d, 1H), 8.28 (dd, 1H), 7.86 (d, 1H), 7.82 (d, 1H), 7.54 (t, 1H), 7.30 (t, 1), 5.20 (sept, 1H), 1.62 (d, 6H).

Method 32

6-Bromo-1,4-dimethyl-9-isopropyl-9H-carbazole

To a solution of 18-crown-6 (0.4 g) in DMF (25 ml) was added potassium bis(trimethylsilyl)amide (2.8 g, 14 mmol). The mixture was stirred at room temperature for 10 minutes. 6-Bromo-1,4-dimethylcarbazole (Chem. Pharm. Bull., 1987, 35 (1), 425-8; 3.5 g, 13 mmol) was added portionwise over 10 minutes and the mixture was then stirred at room temperature for 30 minutes. 2-Iodopropane (1.4 ml, 14 mmol) was added in a single portion and the mixture heated to 90° C. for 18 hours. The solvent was removed in vacuo and the residue partitioned between DCM (50 ml) and 1M HCl (50 ml). The organic layer was separated, washed with brine (50 ml), dried and evaporated in vacuo to leave a red oil. The crude product was purified by flash chromatography eluting with a 5:1 mixture of isohexane: EtOAc to give the product as a colourless solid (1.0 g). NMR (CDCl₃) 8.2 (d, 1H), 7.4 (d, 1H), 7.3 (dd, 1H), 7.0 (d, 1H), 6.8 (d, 1H), 5.4 (m, 1H), 2.7 (s, 3H), 2.65 (s, 3H), 1.6 (d, 6H).

Method 33

6-Bromo-1,4-dimethyl-9-isopropyl-3-nitro-9H-carbazole

6-Bromo-1,4-dimethyl-9-isopropyl-9H-carbazole (Method 32; 1.0 g, 3.2 mmol) was dissolved in glacial acetic acid (20 ml) and the solution cooled in an ice-bath. Concentrated nitric acid (d=1.42, 0.4 ml) was added dropwise over 5 minutes and the mixture was stirred in an ice-bath for 1 hour. The mixture was poured into water (50 ml) and the solid collected by filtration. The solid was washed with water (2×25 ml) and MeOH (2×25 ml). The product was dried in air to leave a yellow solid. NMR (CDCl₃) 8.35 (d, 1H), 7.8 (s, 1H), 7.55 (d, 1H), 7.5 (dd, 1H), 5.4 (m, 1H), 2.95 (s, 3H), 2.75 (s, 3H), 1.7 (d, 6H).

Methods 34-35

Following the procedure of Method 33 and using the appropriate starting materials the following compounds were made.

| Meth | Compound | NMR | SM |
|---|---|---|---|
| 34 | 3-Nitro-6-fluoro-9- | 1.7(d, 6H), 5.0(m, 1H), | Meth 10 |
| | isopropyl-9H-carbazole | 7.3(d, 1H), 7.5(m, 2H), | |
| | | 7.8(d, 1H), 8.4(d, 1H), | |
| | | 9.0(s, 1H) | |
| 35 | 8-Methyl-3-nitro-6- | 2.6(s, 3H), 7.2(d, 1H), | Meth 42 |
| | fluoro-9H-carbazole | 7.6(d, 1H), 8.0(d, 1H), | |
| | | 8.3(d, 1H), 9.2(s, 1H), | |
| | | 12.0(s, 1H) | |

Method 36

3-Amino-1,4-dimethyl-9-isopropyl-9H-carbazole

Method 37

3-Amino-6-bromo-1,4-dimethyl-9-isopropyl-9H-carbazole

Palladium on carbon (10% wt/wt, 0.4 g) was added to a solution of 6-bromo-1,4-dimethyl-9-isopropyl-3-nitro-9H-carbazole (Method 33; 0.49 g, 1.4 mmol) in EtOAc (25 ml). The mixture was stirred under an atmosphere of hydrogen at room temperature for 72 hours. The mixture was filtered through diatomaceous earth and the residue was washed with DCM (2×25 ml). The solvent was evaporated in vacuo and the residue purified by preparative HPLC eluting with 30-95% MeCN in water (0.1% TFA) to give (i) 3-amino-1,4-dimethyl-9-isopropyl-9H-carbazole, eluted first, as a colourless solid (95 mg). NMR 10.15 (brs, 3H), 8.25 (d, 1H), 7.8 (d, 1H), 7.45 (m, 1H), 7.25 (m, 2H), 5.5 (m, 1H), 2.8 (s, 3H), 2.75 (s, 3H), 1.65 (d, 6H); m/z 253; and (ii) 3-amino-6-bromo-1,4-dimethyl-9-isopropyl-9H-carbazole, eluted second, as a colourless solid (33 mg). NMR (CDCl₃) 8.25 (s, 1H), 7.4 (d, 1H), 7.35 (d, 1H), 5.35 (m, 1H), 2.65 (s, 3H), 2.55 (s, 3H), 1.6 (d, 6H).

Method 38

3-Nitro-9-(2-methoxy-1-methylethyl)-4-methyl-9H-carbazole

To a stirred solution of 4-methyl-3-nitro-9H-carbazole (Method 29; 450 mg, 1.99 mM) in DMF (6 ml), was added caesium carbonate (1.3 g, 3.98 mM) followed by toluene-4-sulphonic acid 2-methoxy-1-methyl-ethyl ester (Method 39; 550 mg, 2.39 mM). The resulting suspension was heated to 85° C. and stirred for 5 days. After this time the mixture was poured onto cold water (50 ml) and extracted with EtOAc (3×40 ml). The combined organic layers were washed with saturated NaHCO₃ (3×50 ml), 1M HCl, brine (2×50 ml), dried and concentrated under reduced pressure. The resulting brown oil was chromatographed on 10 g Bond Elut column using DCM as the eluent, to give the title compound 305 mg, 1.0 mmol as a yellow solid. NMR 1.6 (d, 3H), 3.0 (s, 1H), 3.75 (m, 1H), 4.05 (m, 1H), 5.25 (m, 1H), 7.3 (t, 1H), 7.55 (t, 1H), 7.75 (d, 1H), 7.85 (d, 1H), 8.0 (d, 1H), 8.3 (d, 1H); m/z 299.

Method 39

Toluene-4-sulphonic Acid 2-methoxy-1-methylethyl Ester.

To a stirred solution of 1-methoxy-2-propanol (3.91 ml, 40 mmol) in pyridine (25 ml) was added tosyl chloride (6.94 g, 36.4 mmol) in portions over 5 minutes. The reaction was stirred at room temp for 24 hours after which time it was poured into 1M HCl (100 ml) and extracted with EtOAc (3×30 ml). The combined organic layers were washed with 1M HCl (3×75 ml), sat NaHCO₃ (2×50 ml), brine (2×5 ml), dried and concentrated under reduced pressure, yielding 7.2 g, 31 mmol of the title compound as a white solid. NMR 1.2 (d, 3H), 2.35 (s, 3H), 3.15 (s, 3H), 2.3 (m, 2H), 4.6 (m, 1H), 7.25 (d, 2H), 7.7 (d, 2H).

Meth d 40

4-Fluoro-2-methyl-phenyl Hydrazine Hydrochloride

The title compound was prepared using the procedure described in J. Het. Chem 1995 v.32 p.5 but using 4-fluoro-2-methyl-aniline in place of o-toluidine. NMR 2.2 (s, 3H), 7.0 (m, 3H), 7.7 (s, 1H), 10.15 (s, 3H).

Method 41

6-Fluoro-8-methyl-1,2,3,4-tetrahydrocarbazole

Cyclohexanone (4.9 g, 50 mmol) was added to a solution of 4-fluoro-2-methyl-phenyl hydrazine hydrochloride Method 40; 10.75 g, 50 mmol) in EtOH (160 ml) and the mixture was heated at reflux overnight. The slurry was evaporated to dryness, the residue taken up in EtOAc, washed with water then saturated brine, dried, evaporated to dryness and purified by vacuum flash chromatography on Merck 60H silica eluting with toluene to give the title compound 7.2 g. NMR (CDCl₃) 1.9 (m, 4H), 2.3 (s, 3H), 2.7 (m, 2H), 2.8 (m, 2H), 6.7 (d, 1H), 7.0 (d, 1H), 7.5 (s, 1H).

Method 42

1-Methyl-3-fluoro-9H-carbazole

The title compound was prepared using the procedure described in J.Med.Chem 1973 v.16 n.4 p.427 but using 6-fluoro-8-methyl-1,2,3,4-tetrahydrocarbazole (Method 41) in place of 6-fluoro-1,2,3,4-tetrahydrocarbazole. NMR (300 MHz, CDCl₃) 2.6 (s, 3H), 7.0 (d, 1H), 7.2 (t, 1H), 7.4 (m, 2H), 7.6 (d, 1H), 7.9 (s, 1H), 8.0 (d, 1H).

Method 43

3-Amino-4-methyl-9-methylsulphonyl-9H-carbazole

A solution of 4-methyl-9-methylsulphonyl-3-nitro-9H-carbazole (Method 44; 245 mg, 0.80 mmol) in EtOAc (10 ml) was stirred for 2 hr with 10% palladium on carbon (138 mg) in an atmosphere of hydrogen. The reaction mixture was filtered and the filtrate was evaporated to dryness to yield an almost colourless gum, 183 mg. NMR (CDCl₃) 8.18 (d, 1H), 8.15 (d, 1H), 7.84 (d, 1H), 7.45 (dd, 1H), 7.38 (dd, 1H), 2.83 (s, 3H), 2.62 (s, 1H); m/z 275.35.

Method 44

4-Methyl-9-methylsulphonyl-3-nitro-9H-carbazole

Sodium hydride (32 mg of a 60% dispersion in mineral oil, 0.80 mmol) was added to a stirred solution of 4-methyl-3-nitro-9H-carbazole (Method 29; 165 mg, 0.73 mmol) in anhydrous DMF (4 ml). The deep red mixture was stirred for 30 min and then added dropwise to a stirred solution of methanesulphonyl chloride (63 μl, 0.80 mmol) in anhydrous DMF (4 ml). The mixture was stirred 1 hr and partitioned between EtOAc and water. The EtOAc solution was washed with brine, dried and evaporated to a yellow solid (245 mg), which was used without purification. NMR 8.38 (d, 1H), 8.16 (d, 1H), 8.10 (s, 2H), 7.66 (dd, 1H), 7.54 (dd, 1H), 3.45 (s, 3H), 2.94 (s, 3H).

Method 45

cis-1-Amino-3-(ethoxycarbonylmethyl)cyclohexane

1-Amino-3-(ethoxycarbonylmethyl)phenyl (Collect. Czech. Chem. Comm., 60 (1995) 4, 659-66; 21 g), glacial acetic acid (500 ml), Adams Catalyst (2 g) were heated at 35-40° C. at 25 psi for 4 hours. The solvent was removed in vacuo and the residue was partitioned between saturated aqueous sodium carbonate (30 ml) and EtOAc (100 ml). The aqueous phase was extracted with a further 3×100 ml of EtOAc. The organic extracts were combined and washed with brine (30 ml), dried and evaporated to dryness to give the title compound as an oil (6.02 g).

Method 46

cis-1-(t-Butoxycarbonylamino)-3-(ethoxycarbonylmethyl)cyclohexane

Di-t-butyl dicarbonate (6.43 g) in toluene (15 ml) was added portionwise to a solution of cis-1-amino-3-(ethoxycarbonylmethyl)cyclohexane (Method 45; 6.07 g) in toulene (10 ml) When the reaction subsided the clear solution was heated on the steam bath for 1 hour. The solution was then evaporated to dryness to yield a colourless oil. This was distilled in vacuo (11° C. 0.5 mmHg) and trituration of the liquid with 40/60 petrol and cooling gave a white solid 2.58 g. EA: C₁₅H₂₇NO₄: calc C, 63.2%; H, 9.5%; N, 4.9%; obtained C, 63.5%; H, 9.7%; N, 4.8%.

Method 47

cis-1-(t-Butoxycarbonylamino)-3-(carboxymethyl)cyclohexane

cis-1-(t-Butoxycarbonylamino)-3-(ethoxycarbonylmethyl)cyclohexane (Method 46; 4.47 g), MeOH (100 ml) and sodium hydroxide (2 M, 16 ml) was allowed to stand at room temperature overnight. The solvent was removed in vacuo and the resulting aqueous solution was diluted with water (15 ml) and washed with ether. The aqueous layer was then acidified with concentrated hydrochloric acid (to pH 3) and extracted with EtOAc (2×30 ml). The organic phases were combined and washed with brine, dried and evaporated to dryness to give a colourless oil. This was dissolved in ether (20 ml) and was triturated with 40/60 petrol. The solution was cooled in the freezer and the resulting white crystals was filtered and air dried to yield 2.82 g. EA: C₁₃H₂₃NO₄: calc C, 60.7%; H, 8.9%; N, 5.4%; obtained C, 61.0%; H, 9.2%; N, 5.4%.

Method 48

3-(Phenoxycarbonylamino)-2,4-dimethyl-9-isopropyl-9H-carbazole

3-Amino-2,4-dimethyl-9-isopropylcarbazole (Method 14; 2.52 g, 10 mmol) in dry tetrahydrofuran (25 ml) was added to a solution of phenyl chloroformate (1.254 ml, 10 mmol) in dry tetrahydrofuran (25 ml). A solution of triethylamine (1.394 ml, 10 mmol) in dry tetrahydrofuran (5 ml) was added slowly and the reaction mixture allowed to stir at room temperature under an argon atmosphere for 40 minutes. A white precipitate was filtered off and washed with ethyl acetate. The filtrate solvent was removed in vacuo to yield a brown oil. Trituration with iso-hexane gave the title compound as a brown solid (3.651 g, 98%). Rf 0.3. (1:4; ethyl acetate:iso-hexane); NMR 1.62 (d, 6H), 2.47 (s, 3H), 2.73 (s, 3H), 5.10 (sept, 1H), 7.21 (m, 4H), 7.43 (m, 4H), 7.71 (d, 1H), 8.18 (d, 1H), 9.32 (s, 1H); m/z 373.29.

Method 49

3-(Phenoxycarbonylamino)-4-methyl-9-isopropyl-9H-carbazole

The title compound was prepared from 3-amino-4-methyl-9-isopropylcarbazole (Method 21) by the procedure of Method 48. NMR 1.63 (d, 6H), 2.75 (s, 3H), 5.15 (sept, 1H), 7.24 (m, 4H), 7.42 (m, 4H), 7.57 (d, 1H), 7.73 (d, 1H), 8.22 (d, 1H), 9.60 (s, 1H); m/z 359.33.

Method 50

3-(4-Nitrophenoxycarbonylamino)-2,4-dimethyl-9-isopropyl-9H-carbazole

3-Amino-2,4-dimethyl-9-isopropylcarbazole (Method 14; 2.27 g, 9.01 mmol) was added portion-wise to a suspension of potassium carbonate (1.37 g, 9.91 mmol) and 4-nitrophenyl chloroformate (2.0 g, 9.92 mmol) in ethyl acetate (100 ml). After complete addition, the reaction mixture was stirred vigorously for ½ hour. Ethyl acetate (500 ml) was added to the reaction mixture which was then washed with water (3×200 ml). The ethyl acetate was dried, concentrated in vacuo to give yellow solid. This was pre-absorbed onto silica, loaded onto a column and eluted with 1) isohexane 2) 10% diethyl ether/isohexane 3) 50% diethyl ether/isohexane 4) 50% ethyl acetate/isohexane. The required fractions were combined and concentrated in vacuo to give the title compound, 1.6 g (10% unknown impurity by NMR). NMR 1.62 (d, 6H), 2.73 (s, 3H), 5.10 (sept, 1H), 7.18 (t, 1H), 7.4 (t, 1H), 7.7 (d, 1H), 8.05-8.35 (m, 3H).

Method 51

9-Isopropyl-1,2,3,4-tetrahydro-6-nitrocarbazole

9-Isopropyl-1,2,3,4-tetrahydrocarbazole (JCS Perkin 1 573-80, 1983) was nitrated in a similar manner as described for Method 2. NMR (300 MHz, CDCl₃) 8.40 (d, 1H), 8.00 (dd, 1H), 7.39 (d, 1H), 4.64 (m, 1H), 2.75 (m, 4H), 1.97 (m, 2H), 1.88 (m, 2H), 1.60 (d, 6H), 0.88 (m, 1H); m/z 259.7.

Method 52

9-Isopropyl-1,2,3,4-tetrahydro-5-methyl-6-nitrocarbazole

The title compound was prepared from 9-isopropyl-1,2,3,4-tetrahydro-6-nitro-carbazole (Method 51) in a manner similar to that described for Method 25 using methyl magnesium bromide. NMR (300 MHz) 7.58 (d, 1H), 7.48 (d, 1H), 4.70 (m, 1H), 2.96 (m, 2H), 2.85 (m, 2H), 2.84 (s, 3H), 1.78 (m, 4H), 1.50 (d, 6H); m/z 272.

Method 53

9-Isopropyl-1,2,3,4-tetrahydro-5-methyl-6-aminocarbazole Hydrochloride

The title compound was prepared from 9-isopropyl-1,2,3,4-tetrahydro-5-methyl-6-nitrocarbazole (Method 52) in a similar manner as described for Method 14. NMR (300 MHz) 10.0 (br, 3H), 7.38 (d, 1H), 7.08 (d, 1H), 4.64 (m, 1H), 2.93 (m, 2H), 2.73 (m, 2H), 2.53 (s, 3H), 1.77 (m, 4H), 1.48 (d, 6H); m/z 243.4.

### Example 165

The following illustrate representative pharmaceutical dosage forms containing the compound of formula (I), or a pharmaceutically acceptable salt, prodrug or solvate thereof (hereafter compound X), for therapeutic or prophylactic use in humans:-

| (a): Tablet I | mg/tablet |
|---|---|
| Compound X | 100 |
| Lactose Ph.Eur | 182.75 |
| Croscarmellose sodium | 12.0 |
| Maize starch paste (5% w/v paste) | 2.25 |
| Magnesium stearate | 3.0 |

| (b): Tablet II | mg/tablet |
|---|---|
| Compound X | 50 |
| Lactose Ph.Eur | 223.75 |
| Croscarmellose sodium | 6.0 |
| Maize starch | 15.0 |
| Polyvinylpyrrolidone (5% w/v paste) | 2.25 |
| Magnesium stearate | 3.0 |

| (c): Tablet III | mg/tablet |
|---|---|
| Compound X | 1.0 |
| Lactose Ph.Eur | 93.25 |
| Croscarmellose sodium | 4.0 |
| Maize starch paste (5% w/v paste) | 0.75 |
| Magnesium stearate | 1.0 |

| (d): Capsule | mg/capsule |
|---|---|
| Compound X | 10 |
| Lactose Ph. Eur | 488.5 |
| Magnesium stearate | 1.5 |

| (e): Injection I | (50 mg/ml) |
|---|---|
| Compound X | 5.0% w/v |
| 1 M Sodium hydroxide solution | 15.0% v/v |
| 0.1 M Hydrochloric acid | (to adjust pH to 7.6) |
| Polyethylene glycol 400 | 4.5% w/v |
| Water for injection | to 100% |

| (f): Injection II | 10 mg/ml |
|---|---|
| Compound X | 1.0% w/v |
| Sodium phosphate BP | 3.6% w/v |
| 0.1 M Sodium hydroxide solution | 15.0% v/v |
| Water for injection | to 100% |

| (g): Injection III | (1 mg/ml, buffered to pH 6) |
|---|---|
| Compound X | 0.1% w/v |
| Sodium phosphate BP | 2.26% w/v |
| Citric acid | 0.38% w/v |
| Polyethylene glycol 400 | 3.5% w/v |
| Water for injection | to 100% |

Note

The above formulations may be obtained by conventional procedures well known in the pharmaceutical art. The tablets (a)-(c) may be enteric coated by conventional means, for example to provide a coating of cellulose acetate phthalate.

1. A compound of formula (I): 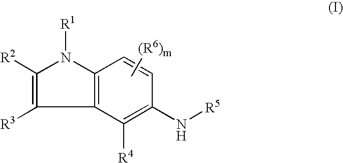
wherein:
R¹ is selected from C₁₋₄alkyl, C₁₋₄alkanoyl, C₁₋₄alkylsulphonyl, N-(C₁₋₄alkyl)sulphamoyl and N,N-(C₁₋₄alkyl)₂sulphamoyl wherein R¹ may be optionally substituted on carbon by one or more R⁷;
R² and R³ are both methyl or R² and R³ together form —(CH₂)₄— or —(CH)₄—; wherein said —(CH₂)₄— or —(CH)₄— may be optionally substituted by R⁸;
R⁴ is C₁₋₄alkyl;
R⁵ is —C(O)NR⁹R¹⁰, —C(O)R⁹ or —C(O)C(O)R⁹;
R⁶ and R⁸ are independently selected from halo, cyano, hydroxy, trifluoromethyl, trifluoromethoxy, C₁₋₄alkyl, N-(C₁₋₄alkyl)amino, N,N-(C₁₋₄alkyl)₂amino and C₁₋₄alkoxy;
R⁷ is halo, nitro, cyano, hydroxy, trifluoromethyl, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, C₁₋₄alkylsulphonylamino, carbocyclyl or heterocyclyl;
R⁹ and R¹⁰ are independently hydrogen, C₁₋₁₀alkyl, C₂₋₁₀alkenyl, C₂₋₁₀alkynyl, C₁₋₄alkoxy, carbocyclyl or heterocyclyl wherein R⁹ and R¹⁰ independently may be optionally substituted on carbon by one or more R¹¹; and wherein if said heterocyclyl contains an —NH— moiety that nitrogen may be optionally substituted by R¹²;
or R⁹ and R¹⁰ together with the nitrogen to which they are attached form a heterocyclic ring optionally substituted on carbon by one or more R¹³; and wherein if said heterocyclic ring contains an —NH— moiety that nitrogen may be optionally substituted by R¹⁴;
R¹¹ and R¹³ are independently selected from halo, hydroxy, cyano, ureido, amino, nitro, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, C₁₋₄alkanoylamino, C₂₋₆alkenyloxycarbonyl, C₁₋₄alkoxycarbonyl, N-(C₁₋₄alkyl)amino, N,N-(C₁₋₄alkyl)₂amino, C₁₋₄alkoxycarbonylamino, C₁₋₄alkoxycarbonyl-N-(C₁₋₄alkyl)amino, N-(C₁₋₄alkyl)carbamoyl, N,N-(C₁₋₄alkyl)₂carbamoyl, C₁₋₄alkylS(O)ₐ wherein a is 0-2, N-(C₁₋₄alkyl)sulphamoyl, N,N-(C₁₋₄alkyl)₂sulphamoyl, heterocyclyl, heterocyclyloxy, heterocyclylcarbonyl, heterocyclylcarbonylamino, heterocyclyloxycarbonyl, heterocyclylthio, carbocyclyl, carbocyclyloxy, carbocyclylcarbonyl, carbocyclylcarbonylamino, carbocyclyloxycarbonyl and carbocyclylthio; wherein R¹¹ and R¹³ independently may be optionally substituted on carbon by one or more R¹⁵; and wherein if said heterocyclyl contains an —NH— moiety that nitrogen may be optionally substituted by R¹⁶;
R¹², R¹⁴ and R¹⁶ are independently selected from C₁₋₄alkyl, C₁₋₄alkanoyl, C₁₋₄alkylsulphonyl, sulphamoyl, N-(C₁₋₄alkyl)sulphamoyl, N,N-(C₁₋₄alkyl)₂sulphamoyl, C₁₋₄alkoxycarbonyl, carbamoyl, N-(C₁₋₄alkyl)carbamoyl, N,N-(C₁₋₄alkyl)₂carbamoyl, carbocyclyl, carbocyclylC₁₋₄alkyl, carbocyclylcarbonyl, carbocyclylsulphonyl, heterocyclyl, heterocyclylC₁₋₄alkyl, heterocyclylcarbonyl, heterocyclylsulphonyl; wherein R¹², R¹⁴ and R¹⁶ independently may be optionally substituted on carbon by one or more R¹⁷;
R¹⁵ is selected from halo, hydroxy, cyano, carbamoyl, ureido, amino, nitro, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, C₁₋₄alkanoylamino, C₂₋₆alkenyloxycarbonyl, C₁₋₄alkoxycarbonyl, N-(C₁₋₄alkyl)amino, N,N-(C₁₋₄alkyl)₂amino, C₁₋₄alkoxycarbonylamino, C₁₋₄alkoxycarbonyl-N-(C₁₋₄alkyl)amino, N-(C₁₋₄alkyl)carbamoyl, N,N-(C₁₋₄alkyl)₂carbamoyl, C₁₋₄alkylS(O)ₐ wherein a is 0-2, N-(C₁₋₄alkyl)sulphamoyl, N,N-(C₁₋₄alkyl)₂sulphamoyl, heterocyclyl, heterocyclyloxy, heterocyclylcarbonyl, heterocyclylmethyloxy, heterocyclyloxycarbonyl, carbocyclyl, carbocyclyloxy, carbocyclylcarbonyl, carbocyclylmethyloxy and carbocyclyloxycarbonyl; wherein R¹⁵ may be optionally substituted on carbon by one or more R¹⁸;
R¹⁷ and R¹⁸ is selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;
m is 0-2; wherein the values of R⁶ may be the same or different;
or a pharmaceutically acceptable salt, prodrug or solvate thereof.

2. A compound of formula (I) as claimed in claim 1 wherein R¹ is selected from C₁₋₄alkyl optionally substituted on carbon by one or more R⁷ wherein R⁷ is C₁₋₄alkoxy, or a pharmaceutically acceptable salt, prodrug or solvate thereof.

3. A compound of formula (I) as claimed in either of claim 1 or claim 2 wherein R² and R³ together form —(CH₂)₄— or —(CH)₄— optionally substituted by R⁸; wherein R⁸ is selected from halo or C₁₋₄alkyl, or a pharmaceutically acceptable salt, prodrug or solvate thereof.

4. A compound of formula (I) as claimed in any one of claims 1-3 wherein R⁴ is methyl or isopropyl, or a pharmaceutically acceptable salt, prodrug or solvate thereof.

5. A compound of formula (I) as claimed in any one of claims 1-4 wherein:
R⁵ is —C(O)NR⁹R¹⁰, —C(O)R⁹ or —C(O)C(O)R⁹; wherein
R⁹ and R¹⁰ are independently hydrogen, C₁₋₁₀alkyl, C₁₋₄alkoxy, carbocyclyl or heterocyclyl wherein R⁹ and R¹⁰ independently may be optionally substituted on carbon by one or more R¹¹; and wherein if said heterocyclyl contains an —NH— moiety that nitrogen may be optionally substituted by R¹²;
or R⁹ and R¹⁰ together with the nitrogen to which they are attached form a heterocyclic ring optionally substituted on carbon by one or more R¹³; and wherein if said heterocyclic ring contains an —NH— moiety that nitrogen may be optionally substituted by R¹⁴;
R¹¹ and R¹³ are independently selected from halo, hydroxy, carbamoyl, amino, carboxy, carbamoyl, C₁₋₄alkyl, C₁₋₄alkoxy, C₁₋₄alkanoylamino, C₁₋₄alkoxycarbonyl, N-(C₁₋₄alkyl)amino, N,N-(C₁₋₄alkyl)₂amino, C₁₋₄alkoxycarbonylamino, N-(C₁₋₄alkyl)carbamoyl, N,N-(C₁₋₄alkyl)₂carbamoyl, C₁₋₄alkylS(O)ₐ wherein a is 0 or 2, heterocyclyl, heterocyclyloxy or carbocyclyl; wherein R¹¹ and R¹³ independently may be optionally substituted on carbon by one or more R¹⁵;
R¹² and R¹⁴ are independently selected from C₁₋₄alkyl, C₁₋₄alkanoyl, C₁₋₄alkoxycarbonyl, carbamoyl, N-(C₁₋₄alkyl)carbamoyl, N,N-(C₁₋₄alkyl)carbamoyl, heterocyclyl and carbocyclylC₁₋₄alkyl;
R¹⁵ is selected from hydroxy, amino, C₁₋₄alkoxycarbonylamino, C₁₋₄alkoxy, N,N-(C₁₋₄alkyl)₂amino, heterocyclyl;
or a pharmaceutically acceptable salt, prodrug or solvate thereof.

6. A compound of formula (I) as claimed in any one of claims 1-5 wherein R⁶ is 2-methyl, or a pharmaceutically acceptable salt, prodrug or solvate thereof.

7. A compound of formula (I) as claimed in any one of claims 1-6 wherein m is 0, or m is 1, wherein R⁶ is ortho to the —NHR⁵ substituent.

8. A compound of formula (I) as depicted in claim 1 wherein
R¹ is selected from C₁₋₄alkyl optionally substituted on carbon by one or more R⁷ wherein R⁷ is C₁₋₄alkoxy;
R² and R³ together form —(CH₂)₄— or —(CH)₄— optionally substituted by R⁸; wherein R⁸ is selected from halo or C₁₋₄alkyl;
R⁴ is methyl or isopropyl;
R⁵ is —C(O)NR⁹R¹⁰, —C(O)R⁹ or —C(O)C(O)R⁹; wherein
R⁹ and R¹⁰ are independently hydrogen, C₁₋₁₀alkyl, C₁₋₄oxy, carbocyclyl or heterocyclyl wherein R⁹ and R¹⁰ independently may be optionally substituted on carbon by one or more R¹¹; and wherein if said heterocyclyl contains an —NH— moiety that nitrogen may be optionally substituted by R¹²;
or R⁹ and R¹⁰ together with the nitrogen to which they are attached form a heterocyclic ring optionally substituted on carbon by one or more R¹³; and wherein if said heterocyclic ring contains an —NH— moiety that nitrogen may be optionally substituted by R¹⁴;
R¹¹ and R¹³ are independently selected from halo, hydroxy, carbamoyl, amino, carboxy, carbamoyl, C₁₋₄alkyl, C₁₋₄alkoxy, C₁₋₄alkanoylamino, C₁₋₄alkoxycarbonyl, N-(C₁₋₄alkyl)amino, N,N-(C₁₋₄alkyl)₂amino, C₁₋₄alkoxycarbonylamino, N-(C₁₋₄alkyl)carbamoyl, N,N-(C₁₋₄alkyl)₂carbamoyl, C₁₋₄alkylS(O)ₐ wherein a is 0 or 2, heterocyclyl, heterocyclyloxy or carbocyclyl; wherein R¹¹ and R¹³ independently may be optionally substituted on carbon by one or more R¹⁵;
R¹² and R¹⁴ are independently selected from C₁₋₄alkyl, C₁₋₄alkanoyl, C₁₋₄alkoxycarbonyl, carbamoyl, N-(C₁₋₄alkyl)carbamoyl, N,N-(C₁₋₄alkyl)₂carbamoyl, heterocyclyl and carbocyclylC₁₋₄alkyl;
R¹⁵ is selected from hydroxy, amino, C₁₋₄alkoxycarbonylamino, C₁₋₄alkoxy, N,N-(C₁₋₄alkyl)₂amino, heterocyclyl; and
R⁶ is 2-methyl;
m is 0 or 1;
or a pharmaceutically acceptable salt, prodrug or solvate thereof.

9. A compound of formula (I) as depicted in claim 1 selected from:
3-(morpholinocarbonylamino)-4-methyl-9-isopropyl-9H-carbazole;
3-(3-carbamoylpiperidin-1-ylcarbonylamino)-2,4-dimethyl-9-isopropyl-9H-carbazole;
3-(1,1-dioxotetrahydrothien-3-ylmethylcarbonylamino)-4-methyl-9-isopropyl-9H-carbazole;
3-(morpholinocarbonylamino)-4-methyl-6-fluoro-9-isopropyl-9H-carbazole;
3-(3-carbamoylpiperidin-1-ylcarbonylamino)-4-methyl-9-isopropyl-9H-carbazole;
3-(4-hydroxypiperidin-1-ylcarbonylamino)-4-methyl-6-fluoro-9-isopropyl-9H-carbazole;
3-[3-(N-methylcarbamoyl)piperidin-1-ylcarbonylamino]-2,4-dimethyl-9-isopropyl-9H-carbazole;
3-[1-(N,N-dimethylcarbamoyl)piperidin-4-ylcarbonylamino]-2,4-dimethyl-9-isopropyl-9H-carbazole;
3-[1-(N,N-dimethylcarbamoyl)pyrrolidin-3-ylcarbonylamino]-2,4dimethyl-9-isopropyl-9H-carbazole; and
3-[4-hydroxypiperidin-1-ylcarbonylamino]-2-methyl-9-isopropyl-9H-carbazole;
or a pharmaceutically acceptable salt, prodrug or solvate thereof.

10. A process for preparing a compound of formula (I) or a pharmaceutically acceptable salt or prodrug or solvate thereof as claimed in claim 1 which process (wherein variable groups are, unless otherwise specified, as defined claim 1) comprises of:
Process a): for compounds of formula (I) wherein R⁵ is —C(O)R⁹; reacting an amine of formula (II): 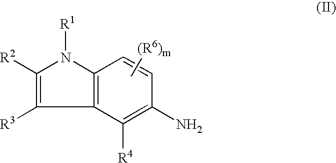
with an acid of formula (III): 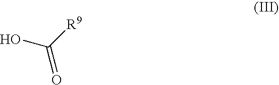
or an activated derivative thereof; or
Process b): for compounds of formula (I) wherein R⁵ is —C(O)NR⁹R¹⁰; by reacting a compound of formula (IV): 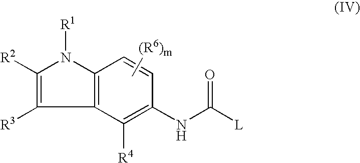
wherein L is a displaceable group; with an amine of formula (V):
HNR⁹R¹⁰ (V)
Process c): for compounds of formula (I) wherein R⁵ is —C(O)NR⁹R¹⁰; reacting a compound of formula (II) with a compound of formula (VI): 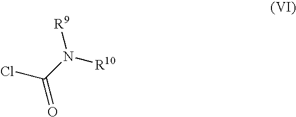
Process d): for compounds of formula (I) wherein R⁵ is —C(O)NR⁹R¹⁰ and one of R⁹ and R¹⁰ is hydrogen; reacting a compound of formula (II) with an isocyanate of formula (VII):
O═—N—R^{a} (VII)
wherein R^{a} is R⁹ or R¹⁰ not equal to hydrogen;
Process e): reacting a compound of formula (VIII): 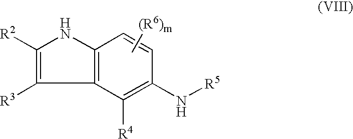
with a compound of formula (IX):
R¹—Z (IX)
wherein Z is a displaceable group or when R¹ is C₁₋₄alkanoyl Z may be hydroxy;
Process f): for compounds of formula (I) wherein R⁵ is —C(O)NR⁹R¹⁰; by reacting a compound of formula (X): 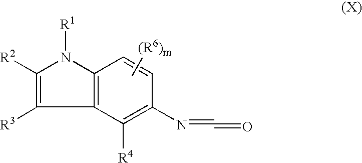
with an amine of formula (V);
and thereafter if necessary:
i) converting a compound of the formula (I) into another compound of the formula (I);
ii) removing any protecting groups;
iii) forming a pharmaceutically acceptable salt, prodrug or solvate thereof.

11. A pharmaceutical composition which comprises a compound of the formula (I) or a pharmaceutically acceptable salt, prodrug or solvate thereof, as claimed in any one of claims 1-9, in association with a pharmaceutically acceptable diluent or carrier.

12. The use of a compound of formula (I), or a pharmaceutically acceptable salt, prodrug or solvate thereof, as claimed in any one of claims 1-9, as a medicament.

13. A method of treating, in a warm-blooded animal, eating disorders, comprising administering a therapeutically effective amount of a compound of formula (I), or a pharmaceutically acceptable salt, prodrug or solvate thereof as claimed in any one of claims 1-9.

14. A compound of formula (I), or a pharmaceutically acceptable salt, prodrug or solvate thereof, as claimed in any one of claims 1-9, in the manufacture of a medicament for the treatment of eating disorders in a warm-blooded animal.

15. The use of a compound of formula (I), or a pharmaceutically acceptable salt, prodrug or solvate thereof, as claimed in any one of claims 1-9, for the treatment of eating disorders in a warm-blooded animal.
